SEQUENCE LISTING (1 sequence)

SEQ ID NO: 1 - protein, 133 aa, Artificial Sequence [sequence omitted]

(19) United States
(12) Patent Application Publication
(10) Pub. No.: US 2021/0205413 A1
(43) Pub. Date: Jul. 8, 2021

(54) SELECTIVE TREG STIMULATOR RUR20kD-IL-2 AND RELATED COMPOSITIONS

(71) Applicant: Nektar Therapeutics, San Francisco, CA (US)

(72) Inventors: Peter Benedict Kirk, Oxfordshire (GB); John L. Langowski, Piedmont, CA (US); Jonathan Zalevsky, Orinda, CA (US)

(21) Appl. No.: 17/056,050

(22) PCT Filed: May 20, 2019
(86) PCT No.: PCT/US2019/033100

§ 371 Date: Nov. 17, 2020

Related U.S. Application Data
(60) Provisional application No. 62/674,244, filed on May 21, 2018.

(51) Int. Cl. A61K 38/20; A61K 9/00; A61K 47/60; A61P 37/06; A61P 37/08

(52) U.S. Cl. CPC A61K 38/2013; A61K 9/0019; A61P 37/08; A61P 37/06; A61K 47/60

(57) ABSTRACT

The instant disclosure provides selective Treg stimulator compositions, including RUR20kD-IL-2 and related compositions, and methods of using these compositions, for example, for treating autoimmune diseases, and/or other conditions responsive to therapy that is effective to provide a selective increase in numbers and activation of regulatory T cells over effector T cells.

The instant application relates to long acting interleukin-2 receptor (IL-2 R) agonist Treg stimulator compositions which selectively increase the number and activation of regulatory T cells, relative to effector T cells, and to methods of using these Treg stimulator compositions in the treatment of autoimmune and inflammatory diseases, and/or other conditions responsive to Treg stimulatory therapy. In particular, the instant application relates to a selective Treg stimulator composition RUR_{20kD}-IL-2 and related compositions, and methods of making the same, formulations thereof, and methods of using RUR_{20kD}-IL-2 and related compositions for the treatment of autoimmune diseases and inflammatory disorders.

The immune system is the body's main line of defense against invasion by infectious organisms. In a normally-functioning immune system, an immune response does not occur against self-antigens; this is referred to as self-tolerance. Autoimmune disease occurs when body tissues are attacked by the body's own immune system due to a loss of tolerance to self-antigens (Dejaco, C., et al., Immunology. 2006; 117(3): 289-300). In subjects having autoimmune disease, body tissues are destroyed by antigen-specific cytotoxic T cells or auto-antibodies, where the accompanying inflammation can cause functional disability and in some cases death. Autoimmune diseases are a heterogeneous collection of diseases with a wide spectrum of symptoms that affect approximately six percent of the population (Siatskas, C., et al., Curr Gene Ther. 2006; 6(1): 45-58). While the clinical features of autoimmune diseases are very different, immune-mediated mechanisms are associated with the generation of an adaptive immune response toward the target antigen (Kuby, J., 1994: Autoimmunity. Immunology, 2^{nd} ed., p 445-467. WH Freeman and Company, New York).

While various conventional treatments, such as corticosteroids, cyclophosphamide, azathioprine, and methotrexate have been marginally effective in some patients with autoimmune disease, they are not uniformly effective and are associated with side effects and toxicity (Jantunen, E., et al, Bone Marrow Transplant. 2000; 25(4): 351-6). Such conventional approaches fail to address the underlying pathology associated with autoreactive immunity. In light of the recent advances in understanding of the pathophysiology of autoimmune diseases, potential new therapies focusing on cellular or molecular targets have been developed and are currently being evaluated. While the etiology of autoimmune disease is unknown, it is believed to be caused by potential interplay between genetic factors, improper immune regulation, and hormonal and environmental factors. Various mechanisms have been proposed for the induction of autoimmune disease including sequestered antigens, molecular mimicry, irregular expression of MHC class II molecules, cytokine imbalance, dysfunction of idiotype network regulatory pathways, general regulatory T cell defects, and polyclonal B cell activation (Kuby, 1994, ibid). Several approaches have been studied for treatment of autoimmune disease, including B cell depletion, anti-cytokine therapy, and stem cell therapy, however these approaches have shortcomings with regard to efficacy, safety and/or undesirable side effects. Conventional therapies for treating autoimmune disease function by suppressing the overall immune system, thereby leading to a significant risk of infection and other serious side effects. Thus, there remains a need for additional treatments to provide an improved combination of efficacy, safety, and/or tolerability for the treatment of autoimmune disease.

For many years, the role of IL-2 in autoimmune responses was established as a pro-inflammatory cytokine. However, more recent studies have suggested that IL-2 can play a protective role in chronic autoimmune inflammation under certain conditions. In particular, a disrupted balance between regulatory T cells (Treg) and effector T cells (Teff) has been identified as a common characteristic of various autoimmune diseases, where such disrupted balance is considered to be affected by homeostatic cytokines such as IL-2. Due to its pharmacokinetic profile, administration of unmodified IL-2 for autoimmune therapy requires frequent daily, or every other day dosing, which is often accompanied by painful injection site reactions. Moreover, the necessity for frequent injections is often accompanied by poor patient compliance due to the discomfort and inconvenience. Long-term repeated administration of IL-2 is also accompanied by an elevated risk of unwanted pleiotropic and systemic activity of IL-2 and associated risks and adverse effects. Further, due to the limited therapeutic window, use of unmodified IL-2 to achieve immune homeostasis and maintenance of the desired Treg/Teff balance may prove challenging, if not unattainable, over prolonged periods of time. Further, its narrow therapeutic margin for autoimmune disease therapy necessitates the administration of extremely low doses of IL-2, thereby adversely affecting its efficacy. While low-dose IL-2 can be used to stimulate Tregs for some clinical benefit, adverse events are dose-limiting, and Treg increases are modest and short-lived. For example, administration of unmodified IL-2 for autoimmune disease therapy induces an undesirable increase in IL-5 and subsequent elevation in eosinophil levels, which can lead to inflammation. Thus, a need remains for agents which may selectively modulate IL-2 signaling in a manner which promotes a disease mitigating balance of regulatory T cells and effector T cell activities in various autoimmune diseases.

Particular autoimmune diseases have underlying etiopathologies, including impaired IL-2 production and/or regulatory T cell deficiencies, which have been implicated as immunological mechanisms preceding the onset of disease. There remains a need for alternative and more effective therapeutic compositions, and treatment regimes, to effectively reduce autoimmune symptoms, improve quality of life, and preferably provide prolonged remission in various autoimmune diseases. The present disclosure addresses the limited availability and related shortcomings of current options for treating chronic autoimmune diseases.

### SUMMARY

The present disclosure is based on the discovery of selective Treg stimulator RUR_{20kD}-IL-2 and related compositions. Selective Treg stimulator compositions of RUR_{20kD}-IL-2 are IL-2-PEG conjugate mixtures of defined heterogeneity. They are intended for low dose subcutaneous administration to selectively restore Treg homeostasis with minimal impact on other immune cells. RUR_{20kD}-IL-2 selective Treg stimulator compositions are mixtures of conjugates comprising recombinant human interleukin-2 (rhIL-2, and in particular the aldesleukin amino acid sequence with no additional amino acid mutations or substitutions), stably covalently conjugated to 20 kDa polyethylene glycol (PEG) moieties, wherein the mixtures have defined fractions with certain degrees of PEGylation per IL-2 moiety. Compositions of the present disclosure comprise selected mixtures of IL-2 PEG conjugates having defined fractions of predominantly di-PEGylated and tri-PEGylated IL-2, and defined lesser fractions of mono-PEGylated IL-2, and/or tetra or higher PEGylated IL-2. In particular, compositions of the present disclosure provide selective Treg stimulator RUR_{20kD}-IL-2 and related compositions, methods of making the same, formulations thereof, and methods of using the RUR_{20kD}-IL-2 and related compositions for the treatment of autoimmune diseases and inflammatory disorders. RUR_{20kD}-IL-2 compositions induce durable responses in immune inflammatory disorders by activating and expanding antigen specific T regulatory cells. Treatment of autoimmune disorders with low dose subcutaneous administration of an RUR_{20kD}-IL-2 composition may provide a means to selectively restore Treg homeostasis, with minimal impact on conventional T cell function, thereby providing an alternative and/or improved approach to alleviate these disorders.

### BRIEF DESCRIPTION OF THE DRAWINGS

FIGS. 1A and 1B are representative reverse phase HPLC plots illustrating the general composition of an RUR_{20kD}-IL-2 composition, the preparation of which is described in Examples 1 and 1A. Moving from left to right along the x-axis (elution times, minutes), the purified conjugate composition comprises primarily di-PEGylated and tri-PEGylated rIL-2.

FIG. 2 is the amino acid sequence of aldesleukin (125-L-serine-2-133 interleukin-2, a recombinant non-glycosylated interleukin-2 expressed in E. coli).

FIGS. 3A and 3B are plots demonstrating the results of a pharmacodynamic analysis of mouse Tregs in blood (FIG. 3A) and spleen (FIG. 3B) following administration of a single-dose of an RUR_{20kD}-IL-2 composition in mice as described in Example 2.

FIGS. 4A, 4B and 4C are plots showing levels of NK cells, CD4 T cells, and CD8 T cells, respectively, in blood, following administration of a single-dose of an RUR_{20kD}-IL-2 composition in mice as described in Example 2.

FIGS. 5A and 5B are plots of Treg function and activity as measured by the mean fluorescence intensity (MFI) of CD25 and Foxp3 following administration of a single-dose of an RUR_{20kD}-IL-2 composition in mice as described in Example 2.

FIGS. 6A-D are plots of splenic Treg isolated from vehicle treated mice at 1 and 4 days in an in vitro Treg suppression assay as described in Example 3.

FIG. 7 is a plot demonstrating the relative suppressive capacity of isolated Treg cultured with Tcon (conventional T cells) at a ratio of 1:2 assessed over time as described in Example 3.

FIGS. 8A and 8B demonstrate the extent of ear swelling in mice treated with an RUR_{20kD}-IL-2 composition; the study was conducted to assess the ability of Treg induction by RUR_{20kD}-IL-2 administration to suppress T-cell antigen-driven inflammation in a mouse model of delayed-type hypersensitivity (DTH) as described in Example 4.

FIGS. 9A-C are plots of Treg levels (CD4, CD25, FOXP3, respectively) in blood following administration of a single dose of an RUR_{20kD}-IL-2 composition in cynomologous monkeys as described in Example 5.

FIGS. 10A and B are plots demonstrating the results of a pharmacodynamic analysis of mouse Tregs following administration of either an RUR_{20kD}-IL-2 composition or unmodified IL-2 (aldesleukin) in mice as described in Example 7.

FIG. 11 is a plot of urine protein levels (g/L) over time for mice administered an RUR_{20kD}-IL-2 composition (0.3 mg/kg) when evaluated in a mouse model of systemic lupus erythematosus (SLE) as described in detail in Example 8.

FIG. 12 is a plot demonstrating the results of a pharmacodynamic analysis of CD4+FoxP3+CD25^{bright} Tregs in peripheral blood (cells/μL) samples over time (days) following a single administration of varying dosage amounts of an RUR_{20kD}-IL-2 composition.

FIG. 13 is a plot demonstrating the results of pharmacodynamic analysis of total CD4+FoxP3+CD25+ Tregs in peripheral blood (cells/μL) samples over time (days) following a single administration varying dosage amounts of an RUR_{20kD}-IL-2 composition to human subjects as described in Example 10.

FIGS. 14A-D are plots of Tcon cell populations, CD4+ (FIG. 14A) and CD8+ Tcon cells (FIG. 14B), expressed as a percentage of CD3 cells, in peripheral blood samples over time (days) following a single administration of varying dosage amounts of an RUR_{20kD}-IL-2 composition to human subjects as described in Example 10. FIGS. 14C and 14D are plots illustrating numbers of CD8+ T cells (cells/μL) and Ki67+CD8+ T cells (expressed as a percentage of CD8), respectively, in peripheral blood samples over time (days) following a single administration of varying dosage amounts of an RUR_{20kD}-IL-2 composition to human subjects as described in Example 10.

FIGS. 15A, 15B are plots of CD25bright+/FoxP3+ Tregs enumerated using flow cytometry. Whole blood was collected from human subjects, pre-treatment and at multiple time points post-treatment with a single administration of varying dosage amounts of RUR_{20kD}-IL-2, as described in Example 10. FIG. 15A illustrates the median peak effect for each dosage amount on numbers (cells/μl) of CD25bright+/FoxP3+ Tregs, while FIG. 15B provides absolute numbers of CD25bright+/FoxP3+ Tregs over time (days) following treatment.

FIGS. 16A, 16B are plots of CD4+ and CD8+ T cells, respectively, enumerated using flow cytometry. Whole blood was collected from human subjects, pre-treatment and at multiple time points post-treatment with a single administration of varying dosage amounts of RUR_{20kD}-IL-2, as described in Example 10. Results are presented as a proportion (%) of each cell population and fold change calculated based on pre-treatment values.

FIGS. 17A, 17B are plots of Treg to Tcon dose-response ratios (FIG. 17A), and CD25bright+/FoxP3+ Tregs and CD8+ T cells (FIG. 17B) enumerated using flow cytometry. Whole blood was collected from human subjects, pre-treatment and at multiple time points post-treatment with a single administration of varying dosage amounts of RUR_{20kD}-IL-2, as described in Example 10. Results are presented as a ratio of the proportion (%) of each cell population and fold change calculated based on pre-treatment values. Tcon cells are CD8+ T cells.

### DETAILED DESCRIPTION

The present disclosure provides selective Treg stimulator compositions, including RUR_{20kD}-IL-2 embodiments and related compositions. Generally, the chemically modified IL-2 conjugate compositions provided herein are characterized by having a particular and predominant number of branched polyethylene glycol moieties stably covalently linked to IL-2 via its amino groups. Compositions provided herein comprise selected mixtures of IL-2 PEG conjugates having defined fractions of predominantly di-PEGylated and tri-PEGylated IL-2, and defined lesser fractions of mono-PEGylated IL-2, and/or tetra or higher PEGylated IL-2.

In one aspect, the present disclosure provides a composition comprising PEGylated IL-2 conjugates having a structure:

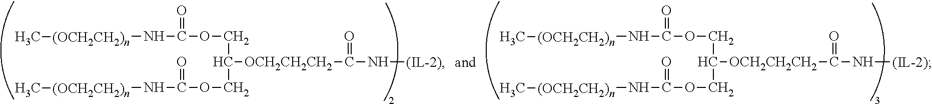

wherein:

IL-2 is an interleukin-2;

n is independently at each occurrence an integer from about 3 to about 4000.

In a particular embodiment of said composition, IL-2 is aldesleukin. In a particular embodiment of said compositions, the nominal average molecular weight of each branched polyethylene glycol moiety is about 20,000 daltons. In a further particular embodiment of said compositions, PEGyalted IL-2 conjugates of the composition have a PEG moiety attached at lysine 31.

In one aspect, provided herein are compositions comprising conjugates of the formula:

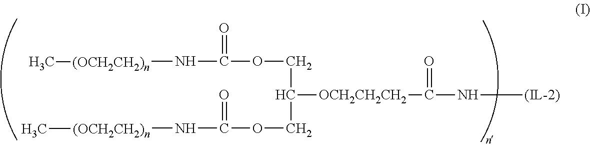

wherein IL-2 is an interleukin-2, n is an integer from about 3 to about 4000, and n′ is 2 and 3. The polymer portion of formula (I) is also referred to as 1,3-bis(methoxypoly(ethylene glycol) MW 10,000 carbamoyl)-2-propanoxy)-4-butanoyl (up to and including the carbonyl group that is covalently attached to an amino nitrogen of the IL-2 moiety). Mixture compositions in accordance with formula (I) are generally referred to herein as RUR-IL2 which encompass a range of PEG sizes. Illustrative ranges of n include, for example, in addition to from about 3 to about 4000, from about 5-2000, or from about 10-1000, or from about 10-750, or from about 10-500, or from about 10-400, or from about 10-300, or from about 10-250, or from about 20-250. In some embodiments, n is, on average, about 226.

In another aspect, provided herein are compositions of the formula:

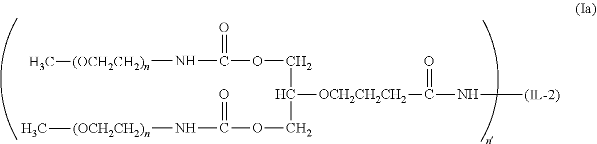

wherein IL-2 is an interleukin-2, n is an integer from about 3 to about 4000, and n′ is 1 and 2 and 3.

In some embodiments, the selective Treg stimulator composition of formula I comprises IL-2R stably covalently-linked with branched polyethylene glycol moieties, where the number of branched PEG moieties per IL-2 moiety (degree of PEGylation) is a distribution of predominantly 2 and 3-mers (di- and tri-PEGylated) in a mixture with minor fractions including 1-mers (mono-PEGylated) and 4-mers (tetra-PEGylated). Thus, in some embodiments minor fractions in the compositions according to formula I will include conjugates wherein n′ is 1, 4, 5, or higher, but not more than 11.

For example, in an embodiment the selective Treg stimulator composition is encompassed by the following structure:

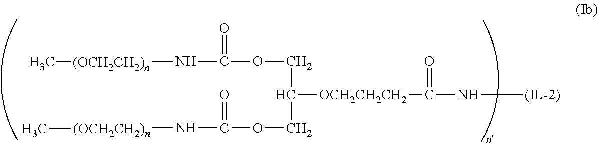

wherein IL-2 is one of the amino acid residues of IL-2, and the “NH” shown in structure (Ib) is an amino group of said IL-2 residue; where “n” is an integer from about 3 to about 4000; and n′ is 2 and 3.

In some embodiments provided herein are selective Treg stimulator compositions referred to as RUR_{20kD}-IL-2 and related compositions. These compositions comprise IL-2 conjugates with individual covalent PEG attachments having nominal molecular weights of about 20 kD total, as described herein. Preferably, the IL-2 moiety is aldesleukin. These compositions further comprise selected mixtures of IL-2 PEG conjugates having defined fractions of predominantly di-PEGylated and tri-PEGylated IL-2, and defined lesser fractions of mono-PEGylated IL-2, and/or tetra or higher PEGylated IL-2. Particular preparations of RUR_{20kD}-IL-2 compositions are described below and throughout this application. As used herein, compositions of RUR_{20kD}-IL-2 of Formula A, compositions of RUR_{20kD}-IL-2 of Formula B, compositions of RUR_{20kD}-IL-2 of Formula C, compositions of RUR_{20kD}-IL-2 of Formula D, and/or compositions of RUR_{20kD}-IL-2 of Formula E, represent certain embodiments of selective Treg stimulator RUR_{20kD}-IL-2 and related compositions, and in these embodiments the IL-2 moiety is aldesleukin (as described herein). Optionally these compositions comprise pharmaceutically acceptable salts thereof.

In an embodiment, provided herein is a composition of RUR_{20kD}-IL-2 of Formula A, wherein the composition comprises, on a molar basis, about 5 mol % or less mono-PEGylated IL-2 conjugates, and from about 28 mol % to about 60 mol % di-PEGylated IL-2 conjugates, and from about 24 mol % to about 65 mol % tri-PEGylated IL-2 conjugates, and about 12 mol % or less of higher PEGylated IL-2 conjugates, and wherein the nominal average molecular weight of each branched polyethylene glycol moiety is about 20,000 daltons. Preferably the composition of RUR_{20kD}-IL-2 of Formula A comprises 80 mol % or greater combined di- and tri-PEGylated IL-2 conjugates.

In an embodiment, provided herein is a composition of RUR_{20kD}-IL-2 of Formula B, wherein the composition comprises, on a molar basis, from about 2.5 to about 4.5 mol % mono-PEGylated IL-2 conjugates, and from about 35 to about 50 mol % di-PEGylated IL-2 conjugates, and from about 38 to about 46 mol % tri-PEGylated IL-2 conjugates, and from about 3 to about 10 mol % higher PEGylated IL-2 conjugates, and wherein the nominal average molecular weight of each branched polyethylene glycol moiety is about 20,000 daltons. Preferably the composition of RUR_{20kD}-IL-2 of Formula B comprises a combined total of di-PEGylated and tri-PEGylated IL-2 conjugates from about 80 to about 95 mol %.

In an embodiment, provided herein is a composition of RUR_{20kD}-IL-2 of Formula C, wherein the composition comprises, on a molar basis, from about 2.8 to about 3.8 mol % mono-PEGylated IL-2 conjugates, and from about 44 to about 48 mol % di-PEGylated IL-2 conjugates, and from about 41 to about 44 mol % tri-PEGylated IL-2 conjugates, and from about 7 to about 9 mol % higher PEGylated IL-2 conjugates, and wherein the nominal average molecular weight of each branched polyethylene glycol moiety is about 20,000 daltons. Preferably the composition of RUR_{20kD}-IL-2 of Formula C comprises a combined total of di-PEGylated and tri-PEGylated IL-2 conjugates from about 87 to about 90 mol %.

In an embodiment, provided herein is a composition of RUR_{20kD}-IL-2 of Formula D, wherein the composition comprises, on a molar basis, from about 2.8 to about 3.8 mol % mono-PEGylated IL-2 conjugates, and from about 44 to about 48 mol % di-PEGylated IL-2 conjugates, and from about 41 to about 44 mol % tri-PEGylated IL-2 conjugates, and from about 7 to about 9 mol % higher PEGylated IL-2 conjugates, and wherein said composition comprises a mixture of mono-PEGylated IL-2 conjugates which have a PEG moiety attached at one of lysine K7 or K8 or K31 or K75, and wherein the nominal average molecular weight of each branched polyethylene glycol moiety is about 20,000 daltons. Preferably the composition of RUR_{20kD}-IL-2 of Formula D comprises a combined total of di-PEGylated and tri-PEGylated IL-2 conjugates from about 87 to about 90 mol %.

In an embodiment, provided herein is a composition of RUR_{20kD}-IL-2 of Formula E, wherein the composition comprises, on a molar basis, from about 2.8 to about 3.8 mol % mono-PEGylated IL-2 conjugates, and from about 44 to about 48 mol % di-PEGylated IL-2 conjugates, and from about 41 to about 44 mol % tri-PEGylated IL-2 conjugates, and from about 7 to about 9 mol % higher PEGylated IL-2 conjugates, and wherein said composition comprises mono-PEGylated IL-2 conjugates which have a PEG moiety attached at lysine K7, wherein the nominal average molecular weight of each branched polyethylene glycol moiety is about 20,000 daltons. Preferably the composition of RUR_{20kD}-IL-2 of Formula E comprises a combined total of di-PEGylated and tri-PEGylated IL-2 conjugates from about 87 to about 90 mol %.

As used herein, “RUR_{20kD}-IL-2 and related compositions” may refer to one or more compositions according to any one of an RUR_{20kD}-IL-2 of Formula A, and/or an RUR_{20kD}-IL-2 of Formula B, and/or an RUR_{20kD}-IL-2 of Formula C, and/or an RUR_{20kD}-IL-2 of Formula D, and/or an RUR_{20kD}-IL-2 of Formula E, and/or pharmaceutically acceptable salts of these compositions. Preparations of Example 1 and/or Example 1A are non-limiting examples of an “RUR_{20kD}-IL-2 and related composition” of the present disclosure.

### Further Embodiments of the Selective Treg Stimulator Compositions Provided Herein:

The compositions provided herein may comprise conjugates where n equals 2, e.g., a di-PEGylated conjugates wherein two branched polyethylene glycol polymers, each having the 1,3-bis(methoxypoly(ethylene glycol)_{10kD}carbamoyl)-2-propanoxy)-4-butanoyl structure shown above, are attached at the same relative locations for substantially all di-PEGylated IL-2 conjugates in the composition. Alternatively, a di-PEGylated conjugate may comprise a mixture of di-PEGylated conjugates, e.g., a mixture of di-PEGylated conjugates where attachment of the branched polyethylene glycol moiety occurs at two sites on IL-2, where the particular attachment sites are not the same for all of the di-PEGylated IL-2 conjugates comprised in the composition. Thus, such di-PEGylated compositions are homogeneous in terms of the degree of PEGylation, in particular the number of branched PEG moieties attached (e.g., 2-mers), but are heterogeneous in terms of the locations of PEG attachment on the IL-2 molecule and in this case represent positional isomers of PEG attachment.

The compositions may also comprise single conjugates where n equals 3, e.g., a tri-PEGylated conjugate wherein three branched polyethylene glycol moieties are attached at the same relative locations for substantially all IL-2 conjugates in the composition. Alternatively, a tri-PEGylated conjugate may comprise a mixture of tri-PEGylated conjugates, e.g., a mixture of tri-PEGylated conjugates where the site of attachment of the branched polyethylene glycol moiety occurs at different sites on IL-2 for the conjugates comprised in the composition. Thus, such tri-PEGylated compositions are homogeneous in terms of the degree of PEGylation, in particular the number of branched PEG moieties attached, but are heterogeneous in terms of the locations of PEG attachment on the IL-2 molecule and in this case represent positional isomers of PEG attachment.

The compositions may also comprise single conjugates where n equals 1, e.g., a mono-PEGylated conjugate wherein one branched polyethylene glycol moieties is attached at the same relative location for substantially all IL-2 conjugates in the composition. Alternatively, a mono-PEGylated conjugate may comprise a mixture of mono-PEGylated conjugates, e.g., a mixture of mono-PEGylated conjugates where the site of attachment of the branched polyethylene glycol moiety occurs at different sites on IL-2 for the conjugates comprised in the composition. Thus, such mono-PEGylated compositions are homogeneous in terms of the degree of PEGylation, in particular the number of branched PEG moieties attached, but are heterogeneous in terms of the location of PEG attachment on the IL-2 molecule and in this case represent positional isomers of PEG attachment.

Certain locations of PEG attachment on the IL-2 molecule are more prevalent in the compositions described herein. For instance, lysines K7 or K8 or K31 or K75, are commonly PEGylated sites. Compositions of RUR_{20kD}-IL-2 and related compositions may comprise conjugates wherein lysines K7 or K8 or K31 or K75 are PEGylated sites. Compositions of RUR_{20kD}-IL-2 and related compositions may comprise mono-PEGylated conjugates wherein lysines K7 or K8 or K31 or K75 are PEGylated sites. Compositions of RUR_{20kD}-IL-2 and related compositions may comprise mono-PEGylated conjugates wherein lysine K7 is a PEGylated site. Compositions of RUR_{20kD}-IL-2 and related compositions may comprise mono-PEGylated conjugates wherein lysine K31 is a PEGylated site.

In some embodiments, the composition contains no more than about 20 mol %, and preferably no more than about 15 mol % of conjugates, when considered collectively, encompassed by formula (I), where n′ is an integer selected from 1, 4, 5, or an integer greater than 5, where the mole percentage is based upon total PEG-IL-2 conjugates. In some embodiments, the composition contains no more than about 10 mol % of conjugates, when considered collectively, encompassed by formula (I), where n′ is an integer selected from 1, 4, 5, or an integer greater than 5, where the mole percentage is based upon total PEG-IL-2 conjugates. In some additional embodiments, the composition contains no more than about 10 mol % of monomers, and preferably no more than about 7 mol % monomers, or no more than about 5 mol percent monomers (i.e., in accordance with structure (I) where n equals 1). In some further embodiments, the composition contains no more than about 10 mol % of tetramers, and preferably no more than about 7 mol % tetramers, or no more than about 5 mol percent tetramers (i.e., in accordance with structure (I) where n equals 4). In certain additional embodiments, the composition comprises no more than about 10 mol % of monomers and no more than about 10 mol % of tetramers. Alternatively, the composition comprises no more than about 7 mol % of monomers and no more than about 7 mol % of tetramers, or may comprise no more than about 5 mol % of monomers and no more than about 5 mol % of tetramers.

In some embodiments, with respect to the PEGylated IL-2 in the composition, the composition will generally satisfy one or more of the following characteristics: at least about 80% of the conjugates in the composition will comprise a mixture of di-PEGylated and tri-PEGylated conjugates, some having 2 and some having 3 branched polymers having the structure shown in formula (I) above attached to the IL-2 moiety; at least about 85% of the conjugates in the composition will comprise a mixture of di-PEGylated and tri-PEGylated conjugates, some having 2 and some having 3 branched polymers having the structure shown in formula (I) above attached to the IL-2 moiety; at least about 90% of the conjugates in the composition will comprise a mixture of di-PEGylated and tri-PEGylated conjugates, some having 2 and some having 3 branched polymers having the structure shown in formula (I) above attached to the IL-2 moiety; and at least about 95% of the conjugates in the composition will comprise a mixture of di-PEGylated and tri-PEGylated conjugates, some having 2 and some having 3 branched polymers having the structure shown in formula (I) above attached to the IL-2 moiety; no more than about 20% of the conjugates in the composition will have from 1, or 4 or more branched polymers having the structure shown in formula (I) above attached to the IL-2 moiety; no more than about 15% of the conjugates in the composition will have from 1, or 4 or more branched polymers having the structure shown in formula (I) above attached to the IL-2 moiety; no more than about 10% of the conjugates in the composition will have from 1, 4 or more branched polymers having the structure shown in formula (I) above attached to the IL-2 moiety; no more than about 7% of the conjugates in the composition will have from 1, or 4 or more branched polymers having the structure shown in formula (I) above attached to the IL-2 moiety.

In some embodiments, the composition contains no more than about 20 mol %, and preferably no more than about 15 mol % of compounds, when considered collectively, encompassed by formula (I), where n′ is an integer selected from 1, 4, 5, or an integer greater than 5, where the mole percentage is based upon total PEG-IL-2 conjugates. In some embodiments, the composition contains no more than about 10 mol % of conjugates, when considered collectively, encompassed by formula (I), where n′ is an integer selected from 1, 4, 5, or an integer greater than 5, where the mole percentage is based upon total PEG-IL-2 conjugates. In some additional embodiments, the composition contains no more than about 10 mol % of monomers, and preferably no more than about 7 mol % monomers, or no more than about 5 mol percent monomers (i.e., in accordance with structure (I) where n equals 1). In some further embodiments, the composition contains no more than about 10 mol % of tetramers, and preferably no more than about 7 mol % tetramers, or no more than about 5 mol percent tetramers (i.e., in accordance with structure (I) where n equals 4). In certain additional embodiments, the composition comprises no more than about 10 mol % of monomers and no more than about 10 mol % of tetramers. Alternatively, the composition comprises no more than about 7 mol % of monomers and no more than about 7 mol % of tetramers, or may comprise no more than about 5 mol % of monomers and no more than about 5 mol % of tetramers.

In some further embodiments, the composition comprises approximately equimolar amounts of

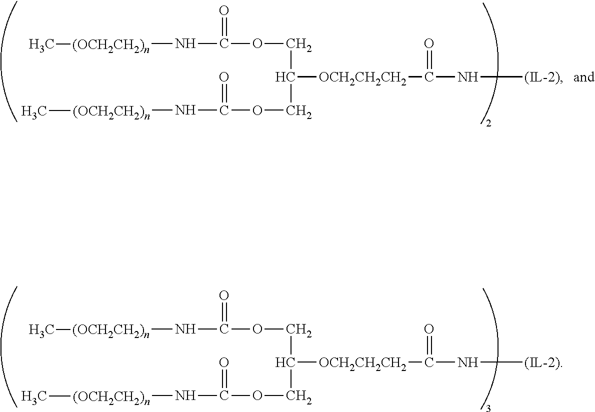

For example, illustrative compositions may comprise any one or more of the following approximate ratios of di-PEGylated species to tri-PEGylated species: 1.4:1; 1.3:1; 1.2:1; 1.1:1; 1:1; 1:1.1; 1:1.2; 1:1.3; or 1:1.4. The average number of PEG moieties per IL-2 for such compositions is selected from, for example, 2; 2.1; 2.2; 2.3; 2.4; 2.5; 2.6; 2.6; 2.7; 2.8; 2.9; and 3. In certain embodiments, the average number of PEG moieties per IL-2 is about 2.5.

For example, in some embodiments, the compositions comprise no more than about 20 mole percent (mol %) of IL-2 conjugates, when considered collectively, encompassed by the formula

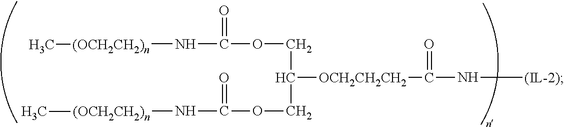

wherein n′ is selected from 1, 4, 5, or an integer greater than 5.

Yet in some additional embodiments, the compositions comprise no more than about 15 mole percent (mol %) of IL-2 conjugates, that when considered collectively, are encompassed by the formula

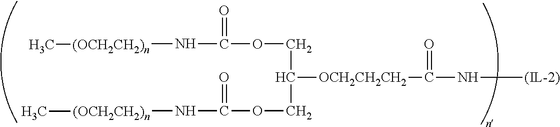

and have n′ selected from 1, 4, 5, or an integer greater than 5.

Yet in some further embodiments, the compositions comprise no more than about 10 mole percent (mol %) of IL-2 conjugates, that when considered collectively, are encompassed by the formula

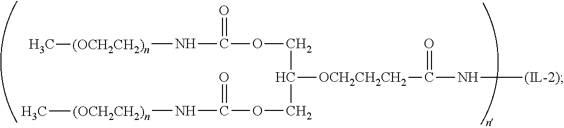

and have n′ selected from 1, 4, 5, or an integer greater than 5.

In some additional embodiments of the foregoing, the composition comprises no more than about 10 mol % of IL-2 conjugates and having n′ equal to 1. In yet some other embodiments, the composition comprises no more than about 7 mol % of IL-2 conjugates having n′ equal to 1.

In yet some further embodiments, the compositions comprise no more than about 5 mol % of IL-2 conjugates n′ equal to 1. In yet some alternative embodiments, the composition comprises less than about 5 mol % of IL-2 conjugates having n′ equal to 1.

In some further embodiments, related to any one or more of the foregoing, the composition comprises no more than about 10 mol % of IL-2 conjugates having n′ equal to 4. Or, in some other embodiments, the composition comprises no more than about 7 mol % of IL-2 conjugates having n′ equal to 4. In yet some further embodiments, the composition comprises no more than about 5 mol % of IL-2 conjugates having n′ equal to 4.

Also provided herein is a composition comprising approximately equimolar amounts of

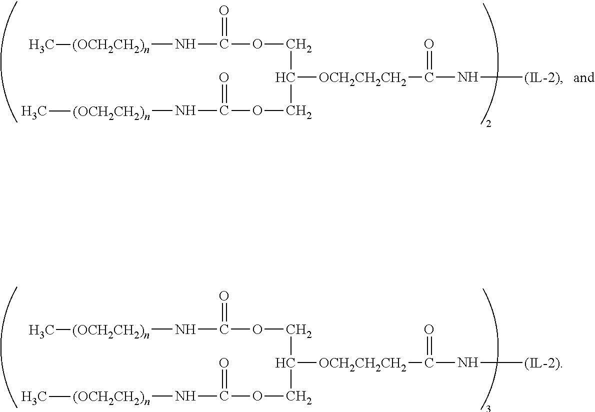

In yet additional embodiments, provided herein is a composition comprising IL-2 conjugates of formula

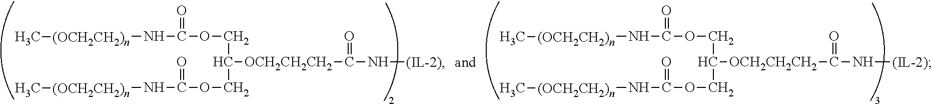

wherein the molar ratio of diPEG/triPEG conjugates is selected from the group consisting of 1.4:1; 1.3:1; 1.2:1; 1.1:1; 1:1; 1:1.1; 1:1.2; 1:1.3; and 1:1.4.

In yet some further embodiments, the composition has an average number of branched polyethylene glycol moieties (having a structure as shown above) per IL-2 residue selected from the group consisting of 2; 2.1; 2.2; 2.3; 2.4; 2.5; 2.6; 2.6; 2.7; 2.8; 2.9; and 3. In a particular embodiment, the average number of branched polyethylene glycol moieties (having a structure as shown above) per IL-2 moiety is about 2.5. In some embodiments related to one or more of the foregoing, the value of n ranges from 5-2000. In some other embodiments, the value of n ranges from 10-1000. In yet some additional embodiments, the value of n ranges from 10-750. In some embodiments the value of n ranges from 10-500, or from 20-250.

The value of n in the embodiments provided herein can vary independently at each occurrence. In one or more embodiments described herein, the value of n in each of the polyethylene glycol arms of the branched polymer is substantially the same. In some further embodiments, the value of n in each of the polymer arms comprising the branched polymer ranges from about 170 to 285. In yet some further embodiments, the value of n in each of the polymer arms comprising the branched polymer ranges from about 204 to about 250. In one or more particular embodiments, the value of n in each of the polymer arms comprising the branched polymer is about 226.

In one or more embodiments related to any one or more of the aspects or embodiments provided herein, the nominal average molecular weight of each branched polyethylene glycol moiety is in a range of from about 250 daltons to about 90,000 daltons. In some other embodiments, the nominal average molecular weight of each branched polyethylene glycol moiety is in a range of from about 1000 daltons to about 60,000 daltons. In yet further embodiments, the nominal average molecular weight of each branched polyethylene glycol moiety is in a range of from about 5,000 daltons to about 60,000 daltons. In some other embodiments, the nominal average molecular weight of each branched polyethylene glycol moiety is in a range of from about 10,000 daltons to about 55,000 daltons.

In yet some additional embodiments, the nominal average molecular weight of each branched polyethylene glycol moiety is in a range of from about 15,000 daltons to about 25,000 daltons. In yet one or more further embodiments, the nominal average molecular weight of each branched polyethylene glycol moiety is in a range of from about 18,000 daltons to about 22,000 daltons. In yet some further embodiments, the nominal average molecular weight of each branched polyethylene glycol moiety is about 20,000 daltons.

Additional exemplary compositions comprise compositions in accordance with the above formulae wherein the overall polymer portion of the molecule has a nominal average molecular weight in a range of from about 250 daltons to about 90,000 daltons. Additional suitable ranges for the polymer portion of the molecule include nominal average molecular weights in a range selected from about 1,000 daltons to about 60,000 daltons, in a range of from about 5,000 daltons to about 60,000 daltons, in a range of about 10,000 daltons to about 55,000 daltons, in a range of from about 15,000 daltons to about 50,000 daltons, and in a range of from about 20,000 daltons to about 50,000 daltons.

Additional illustrative weight-average molecular weights for the polyethylene glycol polymer portion include about 200 daltons, about 300 daltons, about 400 daltons, about 500 daltons, about 600 daltons, about 700 daltons, about 750 daltons, about 800 daltons, about 900 daltons, about 1,000 daltons, about 1,500 daltons, about 2,000 daltons, about 2,200 daltons, about 2,500 daltons, about 3,000 daltons, about 4,000 daltons, about 4,400 daltons, about 4,500 daltons, about 5,000 daltons, about 5,500 daltons, about 6,000 daltons, about 7,000 daltons, about 7,500 daltons, about 8,000 daltons, about 9,000 daltons, about 10,000 daltons, about 11,000 daltons, about 12,000 daltons, about 13,000 daltons, about 14,000 daltons, about 15,000 daltons, about 20,000 daltons, about 22,500 daltons, about 25,000 daltons, about 30,000 daltons, about 35,000 daltons, about 40,000 daltons, about 45,000 daltons, about 50,000 daltons, about 55,000 daltons, about 60,000 daltons, about 65,000 daltons, about 70,000 daltons, and about 75,000 daltons. In some preferred embodiments, the weight-average molecular weight of the branched polyethylene glycol polymer is about 20,000 daltons. In some particular embodiments in which each branched PEG moiety has a nominal molecular weight of about 20,000 daltons, the resulting molecular weight range of the composition is from about 55 to 75 kDa, when characterized for the overall composition.

Further embodiments of the selective Treg stimulator compositions provided herein comprise pharmaceutically acceptable salts thereof. As described above, the IL-2 conjugate compositions may be in the form of a pharmaceutically acceptable salt. Typically, such salts are formed by reaction with a pharmaceutically acceptable acid or an acid equivalent. The term “pharmaceutically acceptable salt” in this respect, will generally refer to the relatively non-toxic, inorganic and organic acid addition salts. These salts can be prepared in situ in the administration vehicle or the dosage form manufacturing process, or by separately reacting a long-acting interleukin-2 composition as described herein with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, oxylate, mesylate, glucoheptonate, lactobionate, and laurylsulphonate salts and the like. (See, for example, Berge et al. (1977) “Pharmaceutical Salts”, J. Pharm. Sci. 66:1-19). Thus, salts as described may be derived from inorganic acids such as hydrochloride, hydrobromic, sulfuric, sulfamic, phosphoric, nitric, and the like; or prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like. As used herein, the term “composition” or “compositions”, including the RUR_{20kD}-IL-2 embodiments and related compositions described herein, comprise any and/or all pharmaceutically acceptable salts of the PEGylated IL-2 conjugates. This description applies whether the term “or pharmaceutically acceptable salt thereof” is added to the description of the composition or not.

### Methods of Use Embodiments

In contrast to unmodified IL-2, the selective Treg stimulator compositions, including RUR_{20kD}-IL-2 embodiments and related compositions described herein, address the underlying pathology associated with autoreactive immunity, as well as target specific mechanisms for producing beneficial T cell functions, and provide significant improvements over administration of unmodified IL-2. To address deficiencies in existing autoimmune disease therapies, the instant compositions provide sustained exposure upon administration and have a unique pharmacological profile. The instant compositions selectively expand and activate endogenous Tregs in vivo, with limited expansion of conventional T cells and/or natural killer cells, and thereby provide a superior approach for the treatment of autoimmune diseases.

More particularly, the selective Treg stimulator compositions, including RUR_{20kD}-IL-2 embodiments and related compositions, provided herein, having a particular and predominant number of branched polyethylene glycol moieties stably covalently linked to IL-2 via its amino groups, have been discovered to be particularly effective when administered at a low doses. The instant compositions are effective in binding and activating the IL-2 receptor to preferentially increase the cell population and immune-suppressive function of regulatory T cells (Treg), while having minimal stimulatory effect on T effector cells (Teff). Sustained exposure to the present compositions (generally referred to herein as RUR_{20kD}-IL-2 and related compositions or in other instances as RUR-IL-2 compositions) in rodent, non-human primate studies, and human clinical studies, was effective to provide a magnitude, duration, and specificity of Treg to Teff responses that could not be achieved with equivalent doses of unmodified IL-2.

Administration of a single low ascending subcutaneous dose of the selective Treg stimulator composition RUR_{20kD}-IL-2 (as described in the supporting examples) to humans resulted in no dose-limiting toxicities, serious adverse events or clinically significant abnormalities. Preliminary pharmacokinetic analysis showed that the composition reached maximum concentrations around about 4-6 days post-dose in most subjects, with little change in concentrations up to approximately 2 weeks post-dose, after which concentrations declined with a half-life of approximately 8-9 days. Preliminary pharmacodynamic assessment revealed that administration of the selective long-acting IL-2 receptor agonist Treg stimulator composition led to a dose-dependent increase in circulating CD4+FoxP3+CD25^{bright} Tregs, i.e., there was a sustained increase in the absolute numbers of circulating CD4+FoxP3+CD25^{bright} Tregs, with levels not returning to baseline until approximately 20 to 25 days following administration. There was a mean increase in the numbers of CD4+FoxP3+CD25^{bright} Tregs of several fold (with magnitude depending upon dose), compared to pre-dose. There was also an increase in the total CD4+FoxP3+CD25+Treg population, although the magnitude of the change was smaller than observed for the CD4+FoxP3+CD25bright Tregs. For the lowest doses, there was no change in the numbers of Tregs in the treated subjects versus placebo subjects. The primary effect was seen on Tregs, as no changes in percentage or numbers of T cell populations (CD4+, CD8+) were observed with an RUR_{20kD}-IL-2 composition at any dose. Thus, the instant compositions and methods are surprisingly effective to increase the suppressive capacity of Treg in in vivo/ex vivo bioassays (even when compared to alternative chemically-modified IL-2 compounds) and in human studies as well, as will be described, along with other features in the sections which follow.

The selective Treg stimulator compositions, including RUR_{20kD}-IL-2 embodiments and related compositions, provided herein, are useful for (among other things) treating autoimmune diseases and disorders. Exemplary autoimmune diseases that can be treated by administration of an RUR-IL-2 or an RUR_{20kD}-IL-2 composition as described herein include systemic conditions such as systemic lupus erythematosus (SLE), ulcerative colitis, Crohn's disease, rheumatoid arthritis, atopic dermatitis, systemic sclerosis, ankylosing spondylitis, graft versus host disease, and polymyositis; or organ-specific autoimmune diseases include type 1 diabetes, Addison's disease, Hashimoto thyroiditis, Graves' disease, Sjogren's syndrome, vitiligo, pernicious anemia, glomerulonephritis, myasthenia gravis, Goodpasture's syndrome, autoimmune hemolytic anemia, ideopathis thrombocytopenia purpura, peanut allergy, and pulmonary fibrosis.

In some embodiments, the condition being treated is systemic lupus erythematosus (SLE). Systemic Lupus Erythematosus (SLE) is an autoimmune inflammatory disease that affects mostly middle-aged women. Characteristics of SLE include, for example, skin eruptions, joint pain, recurrent pleurisy, and kidney disease. A progressive homeostatic imbalance of Tregs relative to Tcons is shared by many autoimmune diseases, including SLE. Taken together, the therapeutic hypothesis relating Treg homeostasis to the pathology of SLE, the activity of low-dose IL-2 in SLE patients, and the superior Treg inducing properties of the RUR_{20kD}-IL-2 and related compositions described herein, relative to IL-2, provide ample support for the use of the instant RUR-IL-2 or RUR_{20kD}-IL-2 and related compositions in treating SLE and other autoimmune diseases and conditions. In one or more further embodiments, provided herein is a method of treating a condition by administering a RUR-IL-2 or RUR_{20kD}-IL-2 related composition as described herein, wherein the condition is selected from the group consisting of, for example, allergy, GVHD, Crohn's disease, ulcerative colitis, rheumatoid arthritis, type-1 diabetes, multiple sclerosis, and psoriasis.

In yet some further embodiments, the RUR-IL-2 or RUR_{20kD}-IL-2 related compositions are effective when administered at a therapeutically effective dose to a subject to preferentially expand and activate regulatory T cells over conventional T cells and natural killer cells.

In another aspect, provided herein is a method of increasing the ratio of regulatory T cells to effector T cells in a subject by administering to the subject a therapeutically effective dose of a RUR-IL-2 or RUR_{20kD}-IL-2 related composition as described herein.

In some embodiments related to the foregoing method, the regulatory T cells are selected from Foxp3+ and CD25+ cells. In one or more embodiments related to the former embodiment or method, the effector T cells are selected from CD4+ and CD8+ cells.

In some further embodiments related to the method or related embodiments above, the fold-increase in regulatory T cells when compared to baseline reaches a value of at least about 2, or at least about 4, or even at least about 6, when evaluated in an in-vivo mouse model.

In some embodiments of the method, the increase in regulatory T cell numbers is sustained above baseline levels for at least 3 days post-administration. In some additional embodiments, the increase in regulatory T cell numbers is sustained above baseline levels for at least 5 days post-administration. Preferably, the increase in regulatory T cell numbers is sustained above baseline levels for at least 7 days.

In yet a further aspect, provided herein is a method of treating a subject having an autoimmune disease, comprising administering to the subject a therapeutically effective amount of a selective Treg stimulator composition, including RUR-IL-2 or RUR_{20kD}-IL-2 related composition embodiments as described above or elsewhere herein.

In yet a further aspect, provided herein is a method of treating a subject having an autoimmune disease, comprising administering to the subject a therapeutically effective amount of a composition selected from the group consisting of: RUR_{20kD}-IL-2 Formula A, RUR_{20kD}-IL-2 Formula B, RUR_{20kD}-IL-2 Formula C, RUR_{20kD}-IL-2 Formula D, and RUR_{20kD}-IL-2 Formula E.

In yet a further aspect, provided herein is a method of treating a subject having an autoimmune disease, comprising administering to the subject a therapeutically effective amount of a composition selected from the group consisting of: RUR_{20kD}-IL-2 Formula A, RUR_{20kD}-IL-2 Formula B, and RUR_{20kD}-IL-2 Formula C.

In yet a further aspect, provided herein is a method of treating a subject having an autoimmune disease, comprising administering to the subject a therapeutically effective amount of a composition of RUR_{20kD}-IL-2 Formula A.

In yet a further aspect, provided herein is a method of treating a subject having an autoimmune disease, comprising administering to the subject a therapeutically effective amount of a composition of RUR_{20kD}-IL-2 Formula B.

In yet a further aspect, provided herein is a method of treating a subject having an autoimmune disease, comprising administering to the subject a therapeutically effective amount of a composition of RUR_{20kD}-IL-2 Formula C.

In yet a further aspect, provided herein is the use in therapy of a composition selected from the group consisting of: RUR_{20kD}-IL-2 Formula A, RUR_{20kD}-IL-2 Formula B, RUR_{20kD}-IL-2 Formula C, RUR_{20kD}-IL-2 Formula D, and RUR_{20kD}-IL-2 Formula E.

In yet a further aspect, provided herein is the use in therapy of a composition of RUR_{20kD}-IL-2 Formula A.

In yet a further aspect, provided herein is the use in therapy of a composition of RUR_{20kD}-IL-2 Formula B.

In yet a further aspect, provided herein is the use in therapy of a composition of RUR_{20kD}-IL-2 Formula C.

In yet a further aspect, provided herein is the use in therapy of a composition of RUR_{20kD}-IL-2 Formula D.

In yet a further aspect, provided herein is the use in therapy of a composition of RUR_{20kD}-IL-2 Formula E.

In yet a further aspect, provided herein is the use of a selective Treg stimulator composition selected from the group consisting of: RUR_{20kD}-IL-2 Formula A, RUR_{20kD}-IL-2 Formula B, RUR_{20kD}-IL-2 Formula C, RUR_{20kD}-IL-2 Formula D, and RUR_{20kD}-IL-2 Formula E, for the manufacture of a medicament for treating autoimmune disease.

In a more particular embodiment, treatment of systemic lupus erythematosus (SLE) comprises subcutaneous administration of a formulation comprising a therapeutically effective amount of RUR-IL-2 or RUR_{20kD}-IL-2 related compositions. See for example, the results described in Example 8, which illustrate the effect of RUR_{20kD}-IL-2 composition-induced Tregs on control of the physiological immune response and disease progression in a representative animal model of SLE. As described therein, an RUR_{20kD}-IL-2 composition was effective to suppress the biomarker of kidney damage (one of the characteristics of patients having SLE) to nearly the same levels as observed in normal mice.

In embodiments that refer to a method of treatment as described herein, such embodiments are also further embodiments for use in that treatment, or alternatively for the use in the manufacture of a medicament for use in that treatment. The present disclosure further provides a composition according to any one of the embodiments of a composition, including formulations thereof, as described herein, for use in therapy. The present disclosure further provides a composition according to any one of the embodiments of a composition, including formulations thereof, as described herein, for use in the treatment of an autoimmune disease.

In one aspect, the present disclosure provides a composition comprising PEGylated IL-2 conjugates having a structure:

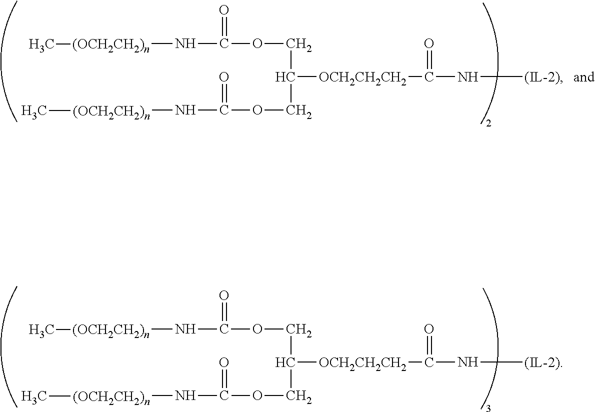

wherein:

IL-2 is an interleukin-2;

n is independently at each occurrence an integer from about 3 to about 4000;

for use in therapy. In a particular embodiment of said composition for use in therapy, IL-2 is aldesleukin. In a particular embodiment of said compositions for use in therapy, the nominal average molecular weight of each branched polyethylene glycol moiety is about 20,000 daltons.

In a further particular embodiment of said compositions for use in therapy, PEGyalted IL-2 conjugates of the composition have a PEG moiety attached at lysine 31. In a particular embodiment of said compositions for use in therapy, therapy is for use in autoimmune disease.

### Terms

In describing and claiming certain features of this disclosure, the following terminology will be used in accordance with the definitions described below unless indicated otherwise.

The term “selective” as used and described herein, refers to an in vivo immunological response which embodies characteristics of induced immune cell, or immunological signal responses, in some respects, but not in others. In particular, “selective” with respect to Treg induction and/or activation refers to an immune response presenting an increase in Treg cell numbers (CD25 high and total by flow cytometry), and/or an increase in the Treg activation state, as indicated by one or more markers of activation, such as ICOS or Ki67 or Stat5, and/or activation refers downstream induced immuno-suppression responses, and/or induced immunological tolerance responses, while lacking certain other immune responses. In this context, “selective Treg induction” refers to an immune response of Tregs as described, while at the same time, lacking significant and/or clinically material effector T cell and associated immunological activation responses. Significant and/or clinically material effector T cell and associated immunological activation responses include for example CD4 positive T effector cell, and/or CD8 positive T effector cell proliferation, and/or markers of activation, such as ICOS or Ki67, or other well-known effector immune responses. Other effector immune response signals may include elevation of certain pro-inflammatory cytokines, such as those known as “cytokine syndrome”, and/or such as IL-5, INF_{gamma}, IL-6, IFNₐₗₚₕₐ, IL-17, IL-22, IL-19. Selective Treg stimulation can also reflected in the mean Treg:Tcon ratio. Preferably the mean Treg:Tcon ratio achieved in response to RUR-IL-2 or RUR_{20kD}-IL-2 related compositions described herein is at least 5 fold, and preferably 7 fold, and more preferably 10 fold or greater.

The term “degree of PEGylation” as used herein refers to the number of stable PEG substituents covalently linked an amino group(s) of an individual aldesleukin polypeptide.

The term “about” as used herein, means in reasonable vicinity of the stated numerical value, such as plus or minus 10% of the stated numerical value. Preferably, “about” or “approximately” as used herein means within plus or minus 5% of a given quantity.

The term “n′ is 2 and 3” as used herein refers to mixtures of IL-2 conjugates wherein the mixtures comprise di-PEGylated and tri-PEGylated conjugates, as described herein.

The term “regulatory T cells” or “Tregs” refer to T cells such CD4+FoxP3+CD25^{bright} phenotypes. (See e.g. Jeffrey A. Bluestone and Qizhi Tang, Treg cells—the next frontier of cell therapy, Science, 12 Oct. 2018⋅Vol. 362 Issue 6411, p 154-155.)

The term “T cons” or “conventional T cells” refer to T lymphocytes that express an αβ T cell receptor (TCR), as well as a co-receptor CD4 or CD8, and carry out well-established adaptive immunity effector functions, such as T helper cell functions and cytotoxic T cell effector functions. For example, Tcon can refer to CD4⁺CD25⁻ naive conventional T cells. “Effector T cells (Teff)” refers to CD4+ and CD8+ cellular effector phenotypes, such as helper T cell, Cytotoxic T cells, and others, as known to the skilled artisan. “NK cells”, also known as “natural killer cells”, “K cells”, or “killer cells” are a type of lymphocyte (white blood cell) and a component of the innate immune system. NK cells play a major role in the host-rejection of tumors and virally infected cells.

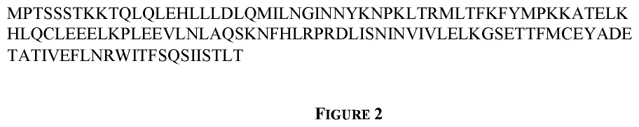

“IL-2 Intermediate” refers to IL-2 polypeptide, in particular aldesleukin. “RUR_{20kD}-IL-2” refers to IL-2 PEG conjugates wherein the IL-2 portion is aldesleukin as described herein, and the PEG portion is as described herein. An RUR_{20kD}-IL-2 composition can also be referred to in a general way by the chemical name (1,3-bis(methoxypoly(ethylene glycol)_{10kD}carbamoyl)-2-propanoxy)-4-butanamide)interleukin-2), recognizing this does not completely describe the composition. As used herein, aldesleukin refers to 125-L-serine-2-133 interleukin-2, a recombinant non-glycosylated interleukin-2 expressed in E. coli. The sequence of amino acid sequence of aldesleukin is shown in FIG. 2. Aldesleukin expressed in other host systems known to the skilled artisan are also within the meaning of the term as used herein.

The term “IL-2” as used herein, refers to a moiety having human IL-2 activity. The term “IL-2 moiety” refers to the IL-2 moiety prior to attachment to a branched polyethylene glycol moiety as well as to the IL-2 moiety following covalent attachment. It will be understood that when the original IL-2 moiety is attached to a polyethylene glycol polymer, such as the branched polyethylene glycol polymer provided herein, the IL-2 moiety is slightly altered due to the presence of one or more covalent bonds associated with linkage to the polyethylene glycol moieties. Such slightly altered form of the IL-2 moiety attached to another molecule is referred to herein as a “residue” of the IL-2 moiety. The term, ‘residue’, in the context of residue of IL-2, means the portion of the IL-2 molecule that remains following covalent attachment to a polymer such as a polyethylene glycol, at one or more covalent attachment sites, as shown in the formulae herein. Typically the site of attachment will be one of 11 amine groups of a lysine in IL-2.

It will be understood that when the unmodified IL-2 is attached to a polymer such as polyethylene glycol, the IL-2 is slightly altered due to the presence of one or more covalent bonds associated with linkage to the polymer(s). This slightly altered form of the IL-2 attached to another molecule such as a branched PEG moiety may be referred to in some instances as a “residue” of the IL-2, or may simply be referred to as “IL-2” or the like, with the understanding that the IL-2 comprised in such polymer conjugate is slightly altered due to the presence of one or more covalent bonds, each linking a branched PEG moiety to the IL-2. The term “higher PEGylated IL-2 conjugates” refers to tetra PEG conjugates or penta PEG conjugates or conjugates up to 11 PEG moieties. Preferably “higher PEGylated IL-2 conjugates” refers to tetra PEG conjugates or penta PEG conjugates.

For example, proteins having an amino acid sequence corresponding to any one of SEQ ID NOs: 1 through 4 described in International Patent Publication No. WO 2012/065086 are exemplary IL-2 proteins, as are any proteins or polypeptides substantially homologous thereto. The term substantially homologous means that a particular subject sequence, for example, a mutant sequence, varies from a reference sequence by one or more substitutions, deletions, or additions, the net effect of which does not result in an adverse functional dissimilarity between the reference and subject sequences. For the purposes herein, sequences having greater than 95 percent homology, equivalent biological activity (although not necessarily equivalent strength of biological activity), and equivalent expression characteristics are considered substantially homologous. For purposes of determining homology, truncation of the mature sequence should be disregarded. As used herein, the term “IL-2” includes such proteins modified deliberately, as for example, by site directed mutagenesis or accidentally through mutations. These terms also include analogs having from 1 to 6 additional glycosylation sites, analogs having at least one additional amino acid at the carboxy terminal end of the protein wherein the additional amino acid(s) includes at least one glycosylation site, and analogs having an amino acid sequence which includes at least one glycosylation site. The term includes both natural and recombinantly produced moieties. In addition, the IL-2 can be derived from human sources, animal sources, and plant sources. One exemplary IL-2 is a human recombinant IL-2 referred to as aldesleukin (See FIG. 2). Reference to a long acting IL-2R agonist as described herein is meant to encompass pharmaceutically acceptable salt forms thereof.

The RUR-IL-2 or RUR_{20kD}-IL-2 related compositions described herein are in one respect long-acting agents. Long-acting, in reference to an RUR-IL-2 or RUR_{20kD}-IL-2 related compositions as provided herein, refers to such composition having a circulating half-life in plasma that is extended over that of the same IL-2R agonist (e.g., aldesleukin or other suitable interleukin-2 sequence) that is unmodified. For example, the comparator agonist is not modified by covalent attachment to one or more water-soluble polymer moieties such as polyethylene glycol moieties, and is compared as administered at a protein equivalent dose of IL-2R agonist to the same subject and assessed by the same pharmacokinetic analysis.

“PEG” or “polyethylene glycol,” as used herein, is meant to encompass any water-soluble poly(ethylene oxide). Unless otherwise indicated, a “PEG polymer” or a polyethylene glycol is one in which substantially all (preferably all) monomeric subunits are ethylene oxide subunits, though, the polymer may contain distinct end capping moieties or functional groups, e.g., for conjugation. PEG polymers for use in the present disclosure will comprise one of the two following structures: “—(CH₂CH₂O)ₙ—” or “—(CH₂CH₂O)ₙ₋₁CH₂CH₂—,” depending upon whether or not the terminal oxygen(s) has been displaced, e.g., during a synthetic transformation. As stated above, for the PEG polymers, the variable (n) ranges from about 3 to 4000, and the terminal groups and architecture of the overall PEG can vary. Preferably PEG has the particular meaning as described in detail herein.

“Branched,” in reference to the geometry or overall structure of a polymer, refers to a polymer having two or more polymer “arms” or “chains” extending from a branch point or central structural feature. As an example, an illustrative PEG reagent, mPEG2-butanoic acid, N-hydroxysuccinimide ester (1,3-bis(methoxypoly(ethylene glycol)carbamoyl)-2-propanoxy)-4-succinimidyl butanoate) is a branched polyethylene glycol polymer comprised of two linear PEG chains, each covalently attached via a carbamate linkage (˜NHC(O)O˜) to the 1- and 3-carbons, respectively, of a central propyl group, from which extends an oxybutanoate succinimidyl ester.

Molecular weight in the context of a water-soluble polymer, such as PEG, can be expressed as either a number (nominal) average molecular weight or a weight average molecular weight. Unless otherwise indicated, all references to molecular weight herein refer to the nominal average molecular weight. Both molecular weight determinations, number average and weight average, can be measured using gel permeation chromatography, gel filtration chromatography, or other liquid chromatography techniques. Other methods for measuring molecular weight values can also be used, such as the use of end-group analysis or the measurement of colligative properties (e.g., freezing-point depression, boiling-point elevation, or osmotic pressure) to determine number average molecular weight or the use of light scattering techniques, ultracentrifugation, or viscometry to determine weight average molecular weight. Gel filtration chromatography is often used to determine the average molecular weight of branched polymers. PEG polymers are typically polydisperse (i.e., number average molecular weight and weight average molecular weight of the polymers are not equal), possessing low polydispersity values of preferably less than about 1.2, more preferably less than about 1.15, still more preferably less than about 1.10, yet still more preferably less than about 1.05, and most preferably less than about 1.03.

A “stable” linkage or bond refers to a chemical bond that is substantially stable in water, that is to say, does not undergo hydrolysis under physiological conditions to any appreciable extent over an extended period of time. Examples of hydrolytically stable linkages generally include but are not limited to the following: carbon-carbon bonds (e.g., in aliphatic chains), ethers, amides, amines, and the like. Generally, a stable linkage is one that exhibits a rate of hydrolysis of less than about 1-2% per day under physiological conditions. Hydrolysis rates of representative chemical bonds can be found in most standard chemistry textbooks.

As used in this specification, the singular forms “a,” “an,” and “the” include plural referents unless the context clearly dictates otherwise.

“Substantially” or “essentially” means nearly totally or completely, for instance, 95% or greater of a given quantity, unless stated to the contrary.

### Preparations and Examples

It should be understood that the Preparations and Examples are set forth by way of illustration and not limitation, and various modifications may be made by one of ordinary skill in the art. Methods of preparing the selective Treg stimulator compositions, including RUR_{20kD}-IL-2 embodiments and related compositions are described herein and/or known to the skilled artisan. The reagents and starting materials are readily available or may be readily synthesized by one of ordinary skill in the art. Suitable conditions for the steps of these methods are well known, and appropriate substitutions of buffers and reagents are within the skill of the art. Furthermore, the skilled artisan will appreciate that in some circumstances the steps and order by which compositions are produced may be modified and is well appreciated by the skilled biochemist. Likewise, it will be appreciated that preparations may be isolated and/or purified by various well-known techniques as needed or desired.

### Preparation of IL-2 Intermediate:

The IL-2 moiety can be derived from non-recombinant methods and/or from recombinant methods and the disclosure is not limited in this regard. The IL-2 moiety can be derived from human sources, animal sources, and plant sources. For example, it is possible to isolate IL-2 from biological systems and otherwise obtain IL-2 from cultured media. See, for example, the procedures described in U.S. Pat. No. 4,401,756 and in Pauly et al. (1984) J. Immunol Methods 75(1):73-84.

Methods for producing and expressing recombinant polypeptides in vitro and in prokaryotic and eukaryotic host cells are well-known to those of ordinary skill in the art. See, for example, U.S. Pat. No. 5,614,185. The IL-2 moiety can be expressed in bacterial [e.g., E. coli, see, for example, Fischer et al. (1995) Biotechnol. Appl. BioIL-2m. 21(3):295-311], mammalian [see, for example, Kronman et al. (1992) Gene 121:295-304], yeast [e.g., Pichia pastoris, see, for example, Morel et al. (1997) Biochem. J. 328(1):121-129], and plant [see, for example, Mor et al. (2001) Biotechnol. Bioeng. 75(3):259-266] expression systems. Although recombinant based methods for preparing proteins can differ, recombinant methods typically involve constructing the nucleic acid encoding the desired polypeptide or fragment, cloning the nucleic acid into an expression vector, transforming a host cell (e.g., plant, bacteria, yeast, transgenic animal cell, or mammalian cell such as Chinese hamster ovary cell or baby hamster kidney cell), and expressing the nucleic acid to produce the desired polypeptide or fragment. Various methods of protein purification may be employed to purify a composition of the present disclosure and such methods are known in the art and described, for example, in Scopes, Protein Purification: Principles and Practice, 3rd Edition, Springer, NY (1994). To facilitate identification and purification of the recombinant polypeptide, nucleic acid sequences that encode for an epitope tag or other affinity binding sequence can be inserted or added in-frame with the coding sequence, thereby producing a fusion protein comprised of the desired polypeptide and a polypeptide suited for binding.

Depending on the system used to express proteins having IL-2 activity, the IL-2 moiety can be unglycosylated or glycosylated and either may be used. That is, the IL-2 moiety may be unglycosylated or the IL-2 moiety may be glycosylated, and in one or more preferred embodiments the IL-2 moiety is unglycosylated. The IL-2 moiety can also advantageously be modified to include and/or substitute one or more amino acid residues such as, for example, lysine, cysteine and/or arginine, in order to provide facile attachment of the polymer to an atom within the side chain of the amino acid. An example of substitution of an IL-2 moiety is described in U.S. Pat. No. 5,206,344. In addition, the IL-2 moiety can be modified to include a non-naturally occurring amino acid residue. Techniques for adding amino acid residues and non-naturally occurring amino acid residues are well known to those of ordinary skill in the art.

In addition, the IL-2 moiety can advantageously be modified to include attachment of a functional group (other than through addition of a functional group-containing amino acid residue). For example, the IL-2 moiety can be modified to include a thiol group. In addition, the IL-2 moiety can be modified to include an N-terminal alpha carbon. In addition, the IL-2 moiety can be modified to include one or more carbohydrate moieties. In addition, the IL-2 moiety can be modified to include an aldehyde group. In addition, the IL-2 moiety can be modified to include a ketone group. In some embodiments of the disclosure, it is preferred that the IL-2 moiety is not modified to include one or more of a thiol group, an N-terminal alpha carbon, carbohydrate, aldehyde group and ketone group.

Exemplary IL-2 moieties are described in the literature, and in for example, U.S. Pat. Nos. 5,116,943, 5,153,310, 5,635,597, 7,101,965 and 7,567,215 and U.S. Patent Application Publication Nos. 2010/0036097 and 2004/0175337. A preferred IL-2 moiety has the amino acid sequence provided in FIG. 2, and represents the amino acid sequence of aldesleukin as used herein.

In some instances, the IL-2 moiety will be in a “monomer” form, wherein a single expression of the corresponding peptide is organized into a discrete unit. In other instances, the IL-2 moiety will be in the form of a “dimer” (e.g., a dimer of recombinant IL-2) wherein two monomer forms of the protein are associated (e.g., by disulfide bonding) to each other. For example, in the context of a dimer of recombinant human IL-2, the dimer may be in the form of two monomers associated to each other by a disulfide bond formed from each monomer's Cys125 residue.

For any given peptide or protein moiety, or composition, it is possible to determine whether that moiety has IL-2 activity. Various methods for determining the in vitro IL-2 activity are described in the art and herein. An exemplary approach is the CTTL-2 cell proliferation assay described herein. An exemplary approach is also described in Moreau et al. (1995) Mol. Immunol. 32:1047-1056). Briefly, in a non-specific binding assay, a proposed IL-2 moiety or composition is allowed to pre-incubate for one hour at 4° C. in the presence of a cell line bearing a receptor of IL-2. Thereafter, ¹²⁵I-labelled IL-2 is allowed to incubate in the system for three hours at 4° C. Data is expressed as % inhibitory capacity of the proposed IL-2 moiety activity versus wild-type IL-2. Other methodologies known in the art can also be used to assess IL-2 function, including electrometry, spectrophotometry, chromatography, and radiometric methodologies.

### Preparation of Selective Treg Stimulator Compositions, Including RUR_{20kD}-IL-2 Embodiments and Related Compositions:

An exemplary selective Treg stimulator composition of RUR_{20kD}-IL-2 is generally prepared by reacting purified IL-2 with a molar excess of PEG reagent (excess of molar equivalents with respect to IL-2), mPEG2(20 kD)-butanoic acid, N-hydroxysuccinimide ester (or any other suitably activated ester) (1,3-bis(methoxypoly(ethylene glycol) MW 10,000 carbamoyl)-2-propanoxy)-4-succinimidyl butanoate, in a bicine solution at high pH of about 9. The reactants are mixed for about 30 minutes to about 5 hours, or from about 30 minutes to 4 hours, or from about 30 minutes to 2 hours, or from about 30 minutes to 1 hour, generally under mild conditions, e.g., from about 20° C. to about 65° C., or from about 20° C. to about 40° C., or at ambient or room temperature. The reaction is quenched by acidification to low pH by addition of a suitable acid such as acetic acid.

The PEGylated rIL-2 reaction product is then purified by a suitable method such as ion exchange chromatography. For example, when employing ion exchange chromatography, the RUR_{20kD}-IL-2 composition binds to the resin and then is eluted with a suitable gradient, such as a sodium chloride gradient. The chromatography product pool is then concentrated and diafiltered into suitable formulation buffer (for example, sodium acetate buffer with sucrose) using, for example, tangential flow filtration (TFF).

If desired, the product pool may be further separated into positional isomers by reverse phase chromatography using a reverse phase-high performance liquid chromatography (RP-HPLC) using a suitable column (e.g., a C18 column or C3 column, available commercially from companies such as Amersham Biosciences or Vydac), or by ion exchange chromatography using an ion exchange column, e.g., a Sepharose™ ion exchange column available from Amersham Biosciences. Either approach can be used to separate polymer-active agent isomers having the same molecular weight (i.e., positional isoforms).

Selective Treg stimulator compositions, including RUR_{20kD}-IL-2 embodiments and related compositions, can be characterized by various analytical and bioassay techniques described herein and/or known to the skilled artisan, including analytical HPLC, SDS-Page, LCMS, and bioassays such as CTLL-2 proliferation, and Treg induction in-vivo.

### Formulations:

In yet one or more embodiments provided herein is a selective Treg stimulator composition, including RUR_{20kD}-IL-2 embodiments and related compositions, comprising an IL-2 conjugate composition as described herein, and a pharmaceutically acceptable excipient. “Pharmaceutically acceptable excipient” or “pharmaceutically acceptable carrier” refers to a component that may be included in the compositions described herein and causes no significant adverse toxicological effects to a subject. The compositions of the present disclosure are preferably formulated as pharmaceutical compositions administered by any route that makes the composition bioavailable, such as parenteral administration, including intravenous, intramuscular or subcutaneous. Such pharmaceutical compositions and processes for preparing same are well known in the art (See, e.g., Remington: The Science and Practice of Pharmacy (D. B. Troy, Editor, 21st Edition, Lippincott, Williams & Wilkins, 2006)). Optionally, the compositions provided herein may further comprise a pharmaceutically acceptable excipient, and exemplary excipients include, without limitation, those selected from the group consisting of carbohydrates, inorganic salts, antimicrobial agents, antioxidants, surfactants, buffers, acids, bases, amino acids, and combinations thereof. The amount of any individual excipient in the composition will vary depending on the activity of the excipient and particular needs of the composition. Typically, the optimal amount of any individual excipient is determined through experimentation, i.e., by preparing compositions containing varying amounts of the excipient (ranging from low to high), examining the stability and other parameters, and then determining the range at which optimal performance is attained with no significant adverse effects. A carbohydrate such as a sugar, a derivatized sugar such as an alditol, aldonic acid, an esterified sugar, and/or a sugar polymer may be present as an excipient. Specific carbohydrate excipients include, for example: monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol, sorbitol (glucitol), pyranosyl sorbitol, myoinositol, cyclodextrins, and the like. The excipient can also include an inorganic salt or buffer such as citric acid, sodium chloride, potassium chloride, sodium sulfate, potassium nitrate, sodium phosphate monobasic, sodium phosphate dibasic, and combinations thereof. The composition can also include an antimicrobial agent for preventing or deterring microbial growth. Non-limiting examples of antimicrobial agents suitable for one or more embodiments of the present disclosure include benzalkonium chloride, benzethonium chloride, benzyl alcohol, cetylpyridinium chloride, chlorobutanol, phenol, phenylethyl alcohol, phenylmercuric nitrate, thimersol, and combinations thereof. An antioxidant can be present in the composition as well. Antioxidants are used to prevent oxidation, thereby preventing the deterioration of the conjugate or other components of the preparation. Suitable antioxidants for use in one or more embodiments of the present disclosure include, for example, ascorbyl palmitate, butylated hydroxyanisole, butylated hydroxytoluene, hypophosphorous acid, monothioglycerol, propyl gallate, sodium bisulfite, sodium formaldehyde sulfoxylate, sodium metabisulfite, and combinations thereof. A surfactant can be present as an excipient. Exemplary surfactants include: polysorbates, such as “Tween 20” and “Tween 80,” and pluronics such as F68 and F88 (both of which are available from BASF, Mount Olive, N.J.); sorbitan esters; lipids, such as phospholipids such as lecithin and other phosphatidylcholines, phosphatidylethanolamines (although preferably not in liposomal form), fatty acids and fatty esters; steroids, such as cholesterol; and chelating agents, such as EDTA; zinc and other such suitable cations. Acids or bases can be present as an excipient in the composition. Non-limiting examples of acids that can be used include those acids selected from the group consisting of hydrochloric acid, acetic acid, phosphoric acid, citric acid, malic acid, lactic acid, formic acid, trichloroacetic acid, nitric acid, perchloric acid, phosphoric acid, sulfuric acid, fumaric acid, and combinations thereof. Examples of suitable bases include, without limitation, bases selected from the group consisting of sodium hydroxide, sodium acetate, ammonium hydroxide, potassium hydroxide, ammonium acetate, potassium acetate, sodium phosphate, potassium phosphate, sodium citrate, sodium formate, sodium sulfate, potassium sulfate, potassium fumerate, and combinations thereof. One or more amino acids can be present as an excipient in the compositions described herein. Exemplary amino acids in this regard include arginine, lysine and glycine. Additional suitable pharmaceutically acceptable excipients include those described, for example, in the Handbook of Pharmaceutical Excipients, 7^{th} ed., Rowe, R. C., Ed., Pharmaceutical Press, 2012. A preferred formulation of the selective Treg stimulator compositions, including RUR_{20kD}-IL-2 embodiments and related compositions provided herein, is 1.5 mg/ml protein equivalent, 10 mM sodium acetate, 110 mM sodium chloride, 2% sucrose (w/v), pH 5.0. An RUR_{20kD}-IL-2 composition can be stored in sterile single-use polycarbonate bottles of appropriate volume with a polypropylene cap with a silicone liner, supplied sterile and ready-to-use.

### Dosing:

The dosing amount of the selective Treg stimulator compositions, including RUR_{20kD}-IL-2 embodiments and related compositions provided herein, will vary depending on a number of factors, but will optimally be a therapeutically effective dose when the composition is stored in a unit dose container (e.g., a vial). In addition, the pharmaceutical preparation can be housed in a syringe. A therapeutically effective dose can be determined experimentally by repeated administration of increasing amounts of the selective Treg stimulator compositions, including RUR_{20kD}-IL-2 embodiments and related compositions provided herein, in order to determine an amount that produces a clinically desired endpoint as described herein, such as relief of autoimmune symptoms and/or immunosuppression, and/or induction of tolerance.

Preferred dosage amounts are low dosage amounts that are effective to preferentially expand and activate regulatory T cells over conventional T cells and natural killer cells in a subject. Activation of regulatory T cells can be measured by a number of different approaches. For example, given the integral role of STAT5 in IL-2-dependent T cell processes, the detection of increased STAT5 in lymphocytes can be utilized as a key marker of Treg activation. Phenotypically, activation of Treg can also be measured by flow cytometry through increased cell surface IL-2Ra(CD25), and/or increased intracellular expression of the protein forkhead box P3 (Foxp3), a master regulator of the Treg lineage, and/or increased expression of the protein Ki67 which is associated with cell proliferation. Collectively, these markers are linked with the functionality of Treg cells and are often dysregulated in such cells in autoimmune diseases. Herein a preferred detection of Treg cell induction and activation is by flow cytometry. The functionality of Treg can also be assessed through an ex vivo suppression assay, which measures their ability to inhibit the proliferation of conventional T cells. The consequence of Treg mobilization and activation can also be directly measured in vivo using antigen-driven inflammation models.

Administration of the RUR_{20kD}-IL-2 embodiments and related compositions provided herein are typically via injection. Other modes of administration are also contemplated, such as pulmonary, nasal, buccal, rectal, sublingual and transdermal. As used herein, the term “parenteral” includes subcutaneous, intravenous, intra-arterial, intratumoral, intralymphatic, intraperitoneal, intracardiac, intrathecal, and intramuscular injection, as well as infusion injections. In a particular embodiment, injection is subcutaneous. For example, administration to a patient can be achieved through injection of a composition comprising RUR_{20kD}-IL-2 embodiments and related compositions provided herein and a diluent. With respect to possible diluents, a diluent can be selected from, for example, bacteriostatic water for injection, dextrose 5% in water, phosphate-buffered saline, Ringer's solution, lactated Ringer's solution, saline, sterile water, deionized water, and combinations thereof. One of ordinary skill in the art can determine through testing whether two given pharmacological components are compatible together in a given formulation. An exemplary composition for administration to a patient, e.g., a subcutaneous formulation, comprises, e.g., a therapeutically effective dose of RUR_{20kD}-IL-2 embodiments and related compositions provided herein, water, sodium acetate, sodium chloride and sucrose. The liquid composition will have a pH in a range of about 4.5-7.5; or from about 4.5-6.

In certain embodiments, the selective Treg stimulator compositions, including RUR_{20kD}-IL-2 embodiments and related compositions provided herein, are in solid form. Preferred solid forms are those that are solid dry forms, e.g., containing less than 5 percent by weight water, or preferably less than 2 percent by weight water. The solid forms are generally suitable for reconstitution in an aqueous diluent. Preferred solid formulations are stable for at least about 24 months when stored in sealed containers at temperatures from about 0-10° C.

The term “patient,” or “subject” as used herein refers to a living organism suffering from or prone to a condition that can be prevented or treated by administration of a composition as provided herein, such as an autoimmune disease, and includes both humans and animals. Subjects include, but are not limited to, mammals (e.g., murines, simians, equines, bovines, porcines, canines, felines, and the like), and preferably are human. In certain embodiments, the patient, preferably a human, is further characterized with a disease, disorder or condition, such as an autoimmune condition, that would benefit from administration of a composition of the present disclosure.

The term “treatment” or “treating” as used herein refers to the management and care of a patient having a condition for which administration of a composition of the present disclosure is indicated for the purpose of combating or alleviating symptoms and complications of those conditions. Treating includes administering a composition of the present disclosure to a patient in need thereof to prevent the onset of symptoms or complications, alleviating the symptoms or complications, or eliminating the disease, condition, or disorder. For example an autoimmune disorder. Preferably treating includes administering a composition of the present disclosure to a patient in need thereof to result in immunosuppression and/or tolerance. The patient to be treated is an animal, and preferably a human being. Administering as used herein includes either when the patient consumes the composition and/or when the patient is directed to consume the composition.

The phrases “pharmaceutically effective amount” and “pharmacologically effective amount” and “therapeutically effective amount” and “physiologically effective amount” are used interchangeably herein and refer to the amount of an RUR_{20kD}-IL-2 and related composition provided herein that is needed to achieve a desired level of the substance in the bloodstream or target tissue. The precise amount will depend upon numerous factors, such as for example, the particular condition being treated, the intended patient population, individual patient considerations, the components and physical characteristics of the therapeutic composition to be administered, and the like.

Pharmaceutical compositions comprising the compound of the present disclosure may be administered parenterally to patients in need of such treatment. Parenteral administration may be performed by subcutaneous, intramuscular or intravenous injection by means of a syringe, optionally a pen-like syringe, or mechanical driven injector. Alternatively, parenteral administration can be performed by means of an infusion pump. Embodiments of the present disclosure provide pharmaceutical compositions suitable for administration to a patient comprising administering to a patient in need thereof a therapeutically effective amount of a composition of the present disclosure and one or more pharmaceutically acceptable excipients. Such pharmaceutical compositions may be prepared by any of a variety of techniques using conventional excipients for pharmaceutical products which are well known in the art. (Remington's Pharmaceutical Sciences, 21st Edition, University of the Sciences in Philadelphia, Philadelphia, Pa., USA (2006)).

The doses of the selective Treg stimulator compositions, including RUR_{20kD}-IL-2 and related compositions provided herein, as well as the dosing regimen associated with the methods and compositions will vary depending upon the age, weight, and general condition of the subject, as well as the type and status of the condition being treated, the judgment of the health care professional, and the particular selective Treg stimulator composition to be administered.

As used herein, the term “effective amount” refers to the amount or dose of a composition of the present disclosure which upon single or multiple dose administration, to the patient or subject, will elicit the biological or medical response of or desired therapeutic effect on a tissue, system, animal, mammal or human that is being sought by the researcher, veterinarian, medical doctor or other clinician. Preferably an effective amount refers to the amount or dose of a composition of the present disclosure which upon single or multiple administration to the patient or subject will induce a selective Treg cell increase of at least 10 fold over pre dose levels. A dose can include a higher initial loading dose, followed by a lower dose. In one or more instances a therapeutically effective amount of the selective Treg stimulator compositions, including RUR_{20kD}-IL-2 and related compositions provided herein, is an amount encompassed by one or more of the following ranges expressed in amount of IL-2: from about 0.10 to about 700 μg/kg; from about 0.20 to about 650 μg/kg, from about 0.30 to about 600 μg/kg; from about 1.0 to about 550 μg/kg, from about 2.0 to about 500 μg/kg, from about 10 to about 450 μg/kg, from about 25 to about 400 μg/kg, from about 50 to about 350 μg/kg or from about 100 to about 300 μg/kg, including any and all combinations of the foregoing beginning and ending values from each and every of the foregoing ranges. In some embodiments, for example, for treating an autoimmune disease, or a disease or condition which can benefit from the preferential expansion and activation of regulatory T cells over conventional T cells and natural killer cells in a subject, the selective Treg stimulator compositions, including RUR_{20kD}-IL-2 embodiments and related compositions provided herein, is administered at a dose, for example, a dose that is less than or equal to 500 μg/kg. A preferred dose regimen of the present disclosure is wherein an RUR_{20kD}-IL-2 and related composition, and in particular those of Formula A-E, is administered at a dose of between 3-24 μg/kg once every two weeks. Another preferred dose regimen of the present disclosure is wherein an RUR_{20kD}-IL-2 and related composition, and in particular those of Formula A-E, is administered at a dose of between 3-18 μg/kg once every two weeks. Another preferred dose regimen of the present disclosure is wherein an RUR_{20kD}-IL-2 and related composition, and in particular those of Formula A-E, is administered at a dose of between 3-12 μg/kg once every two weeks. Another preferred dose regimen of the present disclosure is wherein an RUR_{20kD}-IL-2 and related composition, and in particular those of Formula A-E, is administered at a dose of between 3-6 μg/kg once every two weeks. Another preferred dose regimen of the present disclosure is wherein an RUR_{20kD}-IL-2 and related composition, and in particular those of Formula A-E, is administered at a dose of 3 μg/kg once every two weeks.

The compositions provided herein are effective to restore homeostatic capacity of the immune system, e.g., have the ability to positively affect diseases in which Treg dysfunction plays a role such as autoimmune diseases, allergy, and graft rejection. In one embodiment, provided herein is a method for selectively expanding endogenous Treg in vivo by administering the selective Treg stimulator compositions, including RUR_{20kD}-IL-2 embodiments and related compositions provided herein. Illustrative dosing ranges include for example, from about 100 μg/kg to about 500 μg/kg, or from about 150 μg/kg to about 450 μg/kg, or from about 175 μg/kg to about 400 μg/kg, or even from about 175 μg/kg to about 350 μg/kg. Preferred doses and dosing regimens are described in the examples provided herein. Suitable doses are effective to achieve a maximal amplification of Treg cells, with a minimal stimulation of Teff cells and NK cells; such can be monitored by collection of peripheral blood for flow cytometric analysis to identify the prevalence of Treg cells, effector CD4+ and CD8+ T cells, and NK cells. Based upon these numbers, dosages can be adjusted appropriately.

Dosage regimens may be adjusted to provide the optimum desired response (e.g., a therapeutic effect). Dosing schedules, for intravenous (i.v.) or non-intravenous administration, localized or systemic, or combinations thereof, typically range from a single bolus dosage or continuous infusion to multiple administrations per day (e.g., every 4-6 hours), or as indicated by a treating physician and the patient's condition. With regard to the frequency and schedule of administering the selective Treg stimulator compositions, including RUR_{20kD}-IL-2 embodiments and related compositions provided herein, one of ordinary skill in the art is able to determine an appropriate dosing regimen. For example, in a treatment cycle, a clinician can decide to administer the composition, either as a single dose or in a series of doses, e.g., over the course of several days or weeks). Based upon the long acting nature of the composition, it is preferred that it is typically administered relatively infrequently (e.g., once every three weeks, once every two weeks, once every 8-10 days, once every week, etc.). Exemplary lengths of time associated with the course of therapy include about one week; about two weeks; about three weeks; about four weeks; about five weeks; about six weeks; about seven weeks; about eight weeks; about nine weeks; about ten weeks; about eleven weeks; about twelve weeks; about thirteen weeks; about fourteen weeks; about fifteen weeks; about sixteen weeks; about seventeen weeks; about eighteen weeks; about nineteen weeks; about twenty weeks; about twenty-one weeks; about twenty-two weeks; about twenty-three weeks; about twenty four weeks; about seven months; about eight months; about nine months; about ten months; about eleven months; about twelve months; about thirteen months; about fourteen months; about fifteen months; about sixteen months; about seventeen months; about eighteen months; about nineteen months; about twenty months; about twenty one months; about twenty-two months; about twenty-three months; about twenty-four months; about thirty months; about three years; about four years and about five years. The treatment methods described herein are typically continued for as long as the clinician overseeing the patient's care deems the treatment method to be effective, i.e., that the patient is responding to treatment, or until related symptoms of the condition subside. Non-limiting parameters that indicate the treatment method is effective may include one or more of the following: increased numbers of regulatory T cells such as CD25+ Treg and FoxP3+ Treg, and/or decreased numbers of NK cells and CD4+ and CD8+ effector cells.

The compositions provided herein are useful for increasing the ratio of regulatory T cells, such as Foxp3+ and CD25+ cells, to effector T cells, such as CD4+ and CD8+ cells, when administered to a subject at a therapeutically effective dose. For example, administration of the selective Treg stimulator compositions, including RUR_{20kD}-IL-2 and related compositions provided herein, may be effective to result in at least a two-fold-increase in regulatory T cells, when compared to baseline and evaluated in an in-vivo mouse model, e.g., such as described herein. The method may also, in some embodiments, be effective to result in at least a four-fold-increase in regulatory T cells, when compared to baseline and evaluated in an in-vivo mouse model, e.g., such as described herein. In some instances, the increase in regulatory T cell numbers is sustained above baseline levels for at least 3 days post-administration, or even for at least 5 days post-administration.

As shown in the accompanying examples, the selective Treg stimulator compositions, including RUR_{20kD}-IL-2 embodiments and related compositions provided herein, when administered within a suitable dosage range, are effective to preferentially increase the cell population and immune-suppressive function of regulatory T cells while having minimal stimulatory effect on T effector cells. In certain embodiments, the selective Treg stimulator compositions, including RUR_{20kD}-IL-2 embodiments and related compositions provided herein, are capable of achieving a sustained exposure for providing a magnitude, duration and specificity of Treg to Teff responses that cannot be attained with equivalent doses of native IL-2.

### EXAMPLES

It is to be understood that the foregoing description as well as the examples that follow are intended to illustrate and not limit the scope of the disclosure(s) provided herein. Other aspects, advantages and modifications within the scope of the disclosure will be apparent to those skilled in the art to which the disclosure pertains.

### Materials and Methods

Recombinant human IL-2 having an amino acid sequence identical to that of aldesleukin (des-alanyl-1, serine-125 human interleukin-2, See FIG. 2) is cloned and expressed and used to prepare the exemplary selective Treg stimulator compositions referred to herein as RUR_{20kD}-IL-2. The sequence excludes amino acid #1 (alanine) from the native mature human IL-2 sequence, and there is a cysteine to serine amino acid mutation at amino acid #125. The first amino acid in the sequence is a methionine for direct bacterial expression (no signal peptide encoded). Upon expression, the N-terminal methionine is removed by the host methionine amino peptidase. A single disulfide linkage is formed between the cysteines at amino acid positions #58 and #105. The protein is not glycosylated as it is derived from E. coli. In some descriptions the conjugated IL-2 compositions can be described in some respects as (1,3-bis(methoxypoly(ethylene glycol)carbamoyl)-2-propanoxy)-4-butanamide)interleukin-2), noting this nomenclature does not fully describe the PEGylation pattern or mixture.

Polyethylene glycol reagent, mPEG2(20 kD)-butanoic acid, N-hydroxysuccinimide ester (1,3-bis(methoxypoly(ethylene glycol)_{10kD}carbamoyl)-2-propanoxy)-4-succinimidyl butanoate (also referred to herein as mPEG2-ru-20K NHS), is prepared as described in Example 2 of U.S. Pat. No. 7,887,789. Appearance: white to off-white granular powder; molecular weight (Mn) 18-22 kDa (due to polymer polydispersity). The structure of 1,3-bis(methoxypoly(ethylene glycol)_{10kD}carbamoyl)-2-propanoxy)-4-succinimidyl butanoate is shown below.

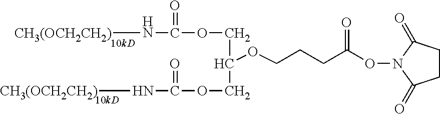

Unless specified otherwise, the concentration, quantity, and dosing levels of the selective Treg stimulator compositions, including RUR_{20kD}-IL-2 embodiments and related compositions provided herein, are reported on a protein basis which only counts the mass contributed by the protein component and not that contributed by the PEG moieties. By using a protein basis, the effective RUR_{20kD}-IL-2 composition molecular weight used for calculations is 15.3 kDa, even for a mixture of conjugated rIL-2 molecules having various degrees of PEGylation, since only the rIL-2 protein is counted.

An RUR_{20kD}-IL-2 related composition is a PEGylated conjugate mixture composition consisting of rhIL-2 (aldesleukin sequence), conjugated to multiple polyethylene glycol (PEG) moieties covalently bound at the lysine groups. The number of PEG moieties per rhIL-2 molecule (degree of PEGylation) is a distribution of predominantly 2 and 3 PEG moieties per molecule (di- or tri-PEGylated) with minor species containing 1 PEG (mono-PEGylated) and 4 PEG (tetra-PEGylated) and/or higher PEGylated molecules, resulting in an average of about 2.5 PEG moieties per rhIL-2. Each PEG moiety has a nominal molecular weight of 20 kDa, and rhIL-2 has a molecular weight of 15.3 kDa, resulting in a nominal RUR_{20kD}-IL-2 molecular weight of 65 kDa.

### Example 1

### Preparation of RUR_{20kD}-IL-2 and Related Compositions

A stock solution (100 mg/mL) of mPEG2-ru-20K NHS is prepared in 2 mM HCl. A typical PEGylation reaction of IL-2 is carried out as follows: 115 mL of IL-2 (aldesleukin) stock solution (1.3 mg/mL) is transferred to a 250 mL plastic bottle and 15 mL of 0.5 M Bicine (N,N-bis(2-hydroxyethyl)glycine), pH 9.2 and 0.5 mL water are added to the solution of IL-2. PEGylation is initiated by drop-wise addition of 19.5 mL of mPEG2-ru-20K NHS stock solution to the IL-2-containing solution. The resultant reaction mixture contains 1 mg/mL IL-2, 50 mM Bicine and 10 molar equivalents of mPEG2-ru-20K NHS (with respect to protein) and has a pH of 8.7. The reaction is allowed to proceed at ambient temperature for 40 min under gentle stirring. The reaction is terminated by adding 2.2 mL acetic acid to reduce the reaction pH to 4.1.

The resulting IL-2 conjugate product is purified by cation exchange chromatography using SP FF Sepharose. Upon completion of the conjugation reaction, the reaction mixture is dialyzed against 20 volumes of 10 mM sodium acetate buffer (pH 4.0). The dialyzed sample is diluted 1:4 with water and loaded onto a column packed with SP FF Sepharose resin. Buffers used for the cation exchange chromatography are as follows: Buffer A: 10 mM sodium acetate (pH 4.0), and Buffer B: 10 mM sodium acetate, 1.0 M sodium chloride (pH 4.0). The resin is washed with Buffer B and equilibrated with Buffer A prior to sample loading. After loading, the resin is washed with 3 column volumes of Buffer A. Conjugated and non-conjugated IL-2 are eluted using a four-step gradient consisting of 0 to 50% Buffer B over 5 column volumes, 25% to 50% Buffer B over 1 column volume, 50% Buffer B over 1 column volume, 50% to 100% Buffer B over 1 column volume and 100% Buffer B over 1 column volume at a flow rate of 28 cm/h. Fractions containing IL-2 conjugates having a degree of PEGylation (dP) of 2 and 3 (i.e., di-mers and tri-mers) are identified by SDS-PAGE and pooled.

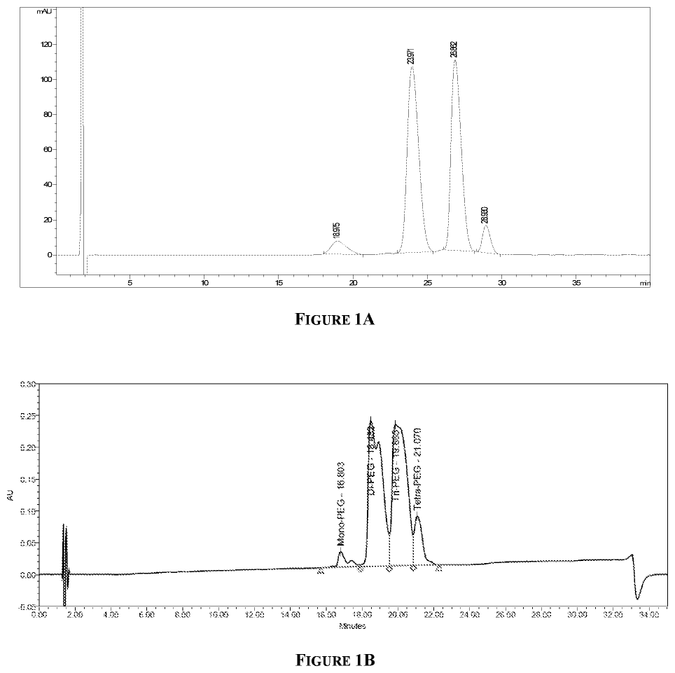

The pooled fractions containing di-mers and tri-mers are concentrated using a stirred ultrafiltration cell (Amicon) and nitrogen gas. The composition of the final product is determined by RP-HPLC using mobile phases: A, 0.09% TFA in water and B, 0.04% TFA in acetonitrile. An Intrada WP-RP C18 column (3×150 mm) is used with a flow rate of 0.5 ml/min and a column temperature of 50° C. The purified conjugate mixture is determined to comprise about 4.6% (mol) of mono-PEGylated rIL-2, about 47.7% (mol) of di-PEGylated rIL-2, about 42.9% (mol) of tri-PEGylated rIL-2 and about 4.8% (mol) of tetra-PEGylated IL-2. See FIG. 1, where elution times are provided on the x-axis. The average degree of PEGylation of the final product mixture is determined to be 2.48 (i.e., about 2.5). No free IL-2 is detected in the final product mixture. This preparation is an example of a composition of RUR_{20kD}-IL-2 of Formula A.

### Example 1-A

### Alternative Preparations of RUR_{20kD}-IL-2 and Related Compositions

Preparation of a desired RUR_{20kD}-IL-2 and related composition consists of: fermentation and purification of the rhIL-2 protein process intermediate, conjugation of rhIL-2 with the PEG reagent starting material mPEG2-ru-20K NHS, purification of IL-2 conjugate fractions having specified degrees of PEGylation, and final formulation of the PEGylated rhIL-2 conjugates to generate the RUR_{20kD}-IL-2 composition of the desired distribution according to the embodiments described herein.

The desired RUR_{20kD}-IL-2 composition is prepared by reacting 1,3-bis(methoxypoly(ethylene glycol)_{10kD}carbamoyl)-2-propanoxy)-4-succinimidyl butanoate (also referred to herein as mPEG2-ru-20K NHS) with lysine residues on the interleukin-2 (IL-2) protein (aldesleukin sequence), resulting in a distribution of PEGylated IL-2 conjugates. The product contains predominately di-PEGylated and tri-PEGylated species, with lower amounts of mono- and/or tetra-PEGylated species.

Frozen IL-2 starting material (purified recombinant IL-2 (aldesleukin sequence) in 10 mM acetate, 5% trehalose, pH 4.5 buffer that had been stored at −70° C.) is thawed to room temperature. The PEG reactant, mPEG2-ru-20K NHS (powder), is solubilized by addition to a 2 mM HCl solution at 90 g/L at room temperature and agitated for a minimum of 15 minutes. The solution is then charged to the reaction vessel. The thawed IL-2 is added to the reaction vessel, diluted appropriately with water, followed by addition of 0.75 M bicine pH 9.7 buffer. The final IL-2 concentration in the reaction mixture is approximately 1.0 g/L, and the bicine concentration is approximately 50 mM to reach a target pH of 8.7. Generally, the PEG:rhIL-2 mass ratio is about 10:1 to 13:1 in a bicine buffered solution at pH 8.5 to 9.5 to PEGylate the protein. The reaction is incubated with continued agitation for 40 minutes at 22° C. as measured from the completion of the mPEG2-ru-20K NHS solution addition. At the end of the incubation period, the reaction is quenched with addition of 1 N acetic acid to rapidly lower the pH, and immediately followed by further stepwise titration to pH 4.0 using additional 1 N acetic acid. The quenched reaction is diluted 10× by addition of water. The diluted quenched reaction is filtered through a 0.22 μm filter to provide crude product.

SP SEPHAROSE® Fast Flow cation exchange chromatography is then conducted on the crude product to partially separate PEGylated reaction fractions. The SP SEPHAROSE® Fast Flow cation exchange chromatography column is equilibrated and the feed loaded at room temperature at a residence time of ˜5 minutes, followed by 5 CV (column volumes) of wash with loading buffer. The PEGylated rhIL-2 binds to the resin while free PEG is washed out. The product is then eluted using a linear gradient elution with 0-500 mM sodium chloride in 10 mM sodium acetate pH 4.0 buffer background. Fractions are collected of 0.15 CV each, starting ˜1 CV into the elution. Fraction collection is ended when absorbance at 280 nm was <5% of peak max. PEGylated fraction concentrations (i.e., mono-PEGylated IL-2 (monomer), di-PEGylated IL-2 (dimer), tri-PEGylated IL-2 (trimer), tetra-PEGylated IL-2 (tetramer), etc., in each of the fractions is measured by absorbance at a wavelength of 280 nm. The distribution of PEGylated fractions is measured by RP-HPLC as described herein, and the fractions containing mono-PEG, di-PEG, tri-PEG, and higher components, are identified, and used to determine the re-pooling of the necessary fractions to generate compositions that will have the target PEGylated fraction distribution profile, as described in an RUR_{20kD}-IL-2 composition as provided herein, and particular in Formulae A-E. Aliquots of selected fractions of identified composition, e.g. di-PEG-IL-2 and tri-PEG-IL-2, and/or mono-PEG or higher PEG, are calculated so as to achieve the target profile as provided herein, and are then re-pooled as needed to obtain an RUR_{20kD}-IL-2 composition having a product with the desired distribution of PEGylated fractions. Alternatively, purification schemes can be devised whereby the elution and collection may provide the desired profile according to the embodiments descried herein without the need for re-pooling. The desired (and/or re-pooled) chromatography purified preparation is then concentrated and diafiltered into 10 mM sodium acetate, 150 mM sodium chloride, 2% w/v sucrose, pH 5.0 using tangential flow filtration (TFF), to achieve a final target concentration of 1 mg/mL (protein basis) of an RUR_{20kD}-IL-2 composition drug substance.

Re-pooled and/or target products are analyzed and the composition distribution is verified by methods described herein, including RP-HPLC, to assess the profile of PEG fractions. Preparations of compositions according to the specifications herein for an RUR_{20kD}-IL-2 composition of Formulae A-E are illustrated by the example product batches numbered 1-4 listed in Table 1 below. Assays for attributes are known to the skilled artisan, and/or described in Examples 1-B through Example 1-I, or otherwise herein. Appropriate historical reference sample compositions are established and are used for comparison in subsequent preparations.

TABLE 1
Summary of Illustrative Analyses of Samples from Different Batches
of an RUR_{20kD}-IL-2 composition by RP-HPLC and SEC-HPLC

| | Fraction | | | | |
|---|---|---|---|---|---|
| | (where | | | | |
| Attribute | applicable) | Batch 1 | Batch 2 | Batch 3 | Batch 4 |
| Appearance of | NA | Clear, | Slightly | Clear, | Clear, |
| sample | | colorless | opalescent, | colorless | colorless |
| | | liquid | colorless liquid | liquid | liquid |
| pH | NA | 5.1 | 5.0 | 5.1 | 5.1 |
| Identity by | NA | Conforms | Conforms to | Conforms to | Conforms to |
| SDS-PAGE | | | reference | reference | reference |
| Identity by | NA | Conforms | Conforms to | Conforms to | Conforms to |
| RP-HPLC | | | reference | reference | reference |
| Protein | NA | 1.58 mg/mL | 0.96 mg/mL | 1.00 mg/mL | 1.12 mg/mL |
| Content by | | | | | |
| BCA | | | | | |
| (vs. rhIL-2) | | | | | |
| Purity | Free IL-2 | <0.1% | ND (not more | ND (not more | ND (not more |
| RP-HPLC | | | than 0.3%) | than 0.3%) | than 0.3%) |
| | Mono-PEG | 3.0% | 3.5% | 3.1% | 3.1% |
| | Di-PEG | 42.4% | 45.8% | 46.8% | 44.4% |
| | Tri-PEG | 46.6% | 42.6% | 42.4% | 44.8% |
| | Higher | 8.0% | 8.1% | 7.7% | 7.7% |
| | PEGylated | | | | |
| | Others | ND | ND (not more | ND (not more | ND (not more |
| | | | than 0.5%) | than 0.5%) | than 0.5%) |
| | Di-PEG/Tri- | 89.0% | 88.4% | 89.2% | 89.2% |
| | PEG Total | | | | |
| Purity | Low | 3.0% | 3.0% | 2.4% | 1.4% |
| SEC-HPLC | Molecular | | | | |
| | Weight | | | | |
| | (Mono) | | | | |
| | Di-PEG | 47.0% | 48.6% | 50.7% | 47.2% |
| | Tri-PEG | 45.0% | 42.5% | 41.2% | 45.6% |
| | High | 4.9% | 5.8% | 5.8% | 5.8% |
| | Molecular | | | | |
| | Weight | | | | |
| | Di-PEG/Tri- | 92.0% | 91.1% | 91.9% | 92.8% |
| | PEG Total | | | | |
| Free PEG | | <0.1% | ND (NMT | ND (NMT | ND (NMT |
| (HPLC/ELSD) | | | 0.1%) | 0.1%) | 0.1%) |

ND is not detectable, NMT is not more than.

In some embodiments the RUR_{20kD}-IL-2 composition product will contain, on a molar basis, less than 1% free, unconjugated IL-2 (more preferably no detectable free IL-2), 5% or less mono-PEGylated IL-2, from 28% to about 60% di-PEGylated IL-2, from about 24% to about 65% tri-PEGylated IL-2, 12% or less of higher PEGylated IL-2 species, and 80% or greater combined di- and tri-PEGylated IL-2 species.

In some embodiments, the RUR_{20kD}-IL-2 composition product will contain, for example, less than 0.5 mol % free IL-2, from about 2.5 to about 4.5 mol % mono-PEGylated IL-2, from about 35 to about 50 mol % di-PEGylated IL-2, from about 38 to about 46 mol % tri-PEGylated IL-2, from about 3 to about 10 mol % higher PEGylated IL-2 species, and a combined total of di-PEGylated and tri-PEGylated IL-2 from about 80 to about 95 mol %.

In some embodiments, the RUR_{20kD}-IL-2 composition product will contain, for example, on a molar basis, 5% or less mono-PEGylated IL-2, and from 28% to about 60% di-PEGylated IL-2, and from about 24% to about 65% tri-PEGylated IL-2, and 12% or less of higher PEGylated IL-2 species. Preferably the composition comprises 80% or greater combined di- and tri-PEGylated IL-2 species.

In some embodiments, the RUR_{20kD}-IL-2 composition product will contain, for example, about 2.5 to about 4.5 mol % comprises mono-PEGylated IL-2, and from about 35 to about 50 mol % comprises di-PEGylated IL-2, and from about 38 to about 46 mol % comprises tri-PEGylated IL-2, and from about 3 to about 10 mol % comprises higher PEGylated IL-2 species. Preferably the composition comprises a combined total of di-PEGylated and tri-PEGylated IL-2 from about 80 to about 95 mol %.

In some embodiments, the RUR_{20kD}-IL-2 composition product will contain, for example, from about 2.8 to about 3.8 mol % comprises mono-PEGylated IL-2, and from about 44 to about 48 mol % comprises di-PEGylated IL-2, and from about 41 to about 44 mol % comprises tri-PEGylated IL-2, and from about 7 to about 9 mol % comprises higher PEGylated IL-2 species. Preferably the composition comprises a combined total of di-PEGylated and tri-PEGylated IL-2 from about 87 to about 90 mol %.

In some embodiments, the RUR_{20kD}-IL-2 composition product will contain, for example, about 2.8 to about 3.8 mol % comprises mono-PEGylated IL-2, and from about 44 to about 48 mol % comprises di-PEGylated IL-2, and from about 41 to about 44 mol % comprises tri-PEGylated IL-2, and from about 7 to about 9 mol % comprises higher PEGylated IL-2 species, and wherein said composition comprises a mixture of mono-PEGylated IL-2 conjugates which have a PEG moiety attached at one of lysine K7 or K8 or K31 or K75. Preferably the composition comprises a combined total of di-PEGylated and tri-PEGylated IL-2 from about 87 to about 90 mol %.

In some embodiments, the RUR_{20kD}-IL-2 composition product will contain, for example, about 2.8 to about 3.8 mol % comprises mono-PEGylated IL-2, and from about 44 to about 48 mol % comprises di-PEGylated IL-2, and from about 41 to about 44 mol % comprises tri-PEGylated IL-2, and from about 7 to about 9 mol % comprises higher PEGylated IL-2 species, and wherein said composition comprises mono-PEGylated IL-2 conjugates which have a PEG moiety attached at lysine K7. Preferably the composition comprises a combined total of di-PEGylated and tri-PEGylated IL-2 from about 87 to about 90 mol %.

### Example 1-B

### Purity and Characterization of an RUR_{20kD}-IL-2 Composition Via Reverse Phase High Performance Liquid Chromatography

Reverse phase high performance liquid chromatography (RP-HPLC) is used to assess the chromatographic purity and identity of samples of an RUR_{20kD}-IL-2 composition using an Agilent 1200 series instrument equipped with a diode array detector (UV at 215 nm). The column used can be an ACE 3 Phenyl-300 column (Mac-Mod Analytical Inc.) (or other suitable column) with an eluent flow rate of 0.6 mL/min. RP-HPLC is carried out using a gradient containing mixtures of two mobile phases: (1) Mobile Phase A, a solution of 0.1% formic acid in water, and (2) Mobile Phase B, a solution of 0.1% formic acid in acetonitrile. The linear gradient ranged from 60% Mobile Phase A/40% Mobile Phase B to 40% Mobile Phase A/60% Mobile Phase B, to 20% Mobile Phase A/80% Mobile Phase B, to 60% Mobile Phase A/40% Mobile Phase B. The components of the diluent/formulation buffer are 10 mM sodium acetate, 200 mM sodium chloride, 2% sucrose, at a pH of 5.0.

Frozen RUR_{20kD}-IL-2 composition reference material and samples are thawed and diluted to 1.0 mg/mL with formulation buffer. At least one blank control of formulation buffer is first subjected to RP-HPLC via injection to ensure no interference with analysis of RUR_{20kD}-IL-2-composition related peaks. Next, RUR_{20kD}-IL-2 composition reference material or control was injected five times. RUR_{20kD}-IL-2 composition samples are next injected. RUR_{20kD}-IL-2 composition reference material/control is injected after every six sample injections and at the end of the injection sequence.

The % relative standard deviation (RSD) of retention time for the first five reference material injections comprising di-PEGylated (di-PEG) and tri-PEGylated (tri-PEG) RUR_{20kD}-IL-2 compositions are not more than 2.0%. The % RSD area percent for all reference material RUR_{20kD}-IL-2 composition injections of the di-PEG and tri-PEG components are not more than 5.0%. All RUR_{20kD}-IL-2 composition peaks from reference and sample injections are integrated. Specifically, for a 1.0 mg/mL concentration of an RUR_{20kD}-IL-2 composition, the di-PEG and tri-PEG RUR_{20kD}-IL-2 composition species above a 0.5% limit of detection (LOD) and the rhIL-2 peak above a 0.3% LOD are respectively integrated. For a 1.0 mg/mL concentration of RUR_{20kD}-IL-2, the limit of quantitation (LOQ) is 1.0% for di-PEG and tri-PEG RUR_{20kD}-IL-2 species and 0.5% for rhIL-2. Results from analyses are shown in Table 2 (6 samples) and Table 3 (12 samples) below.

TABLE 2
Area percent for Mono-PEG, Di-PEG, Tri-PEG, Tetra-PEG, and Penta-PEG fractions
from six RUR_{20 kD}-IL-2 composition replicate samples analyzed by RP-HPLC

| | Result | Mono-PEG | Di-PEG | Tri-PEG | Tetra-PEG | Penta-PEG |
|---|---|---|---|---|---|---|
| Sample Name | Id | (% Area) | (% Area) | (% Area) | (% Area) | (% Area) |
| Sample 1 | 1084 | 2.93 | 42.54 | 46.69 | 7.10 | 0.75 |
| Sample 2 | 1085 | 2.98 | 42.48 | 46.67 | 7.17 | 0.70 |
| Sample 3 | 1086 | 2.98 | 42.60 | 46.65 | 7.21 | 0.56 |
| Sample 4 | 1087 | 3.04 | 42.58 | 46.50 | 7.18 | 0.71 |
| Sample 5 | 1088 | 2.97 | 42.52 | 46.62 | 7.33 | 0.56 |
| Sample 6 | 1089 | 2.88 | 42.51 | 46.69 | 7.34 | 0.58 |

| | | | | | |
|---|---|---|---|---|---|
| Mean | 2.96 | 42.54 | 46.64 | 7.22 | 0.64 |
| % RSD (not more than 2.0% | 1.8 | 0.1 | 0.2 | 1.3 | 13.3 |
| for Di-PEG and Tri-PEG) | | | | | |

TABLE 3
Area percent for Mono-PEG, Di-PEG, Tri-PEG, Tetra-PEG, and Penta-PEG fractions
from twelve RUR_{20 kD}-IL-2 composition replicate samples analyzed by RP-HPLC

| | Result | Mono-PEG | Di-PEG | Tri-PEG | Tetra-PEG | Penta-PEG |
|---|---|---|---|---|---|---|
| Sample Name | Id | (% Area) | (% Area) | (% Area) | (% Area) | (% Area) |
| Sample 7 | 2323 | 3.30 | 42.37 | 46.20 | 7.84 | 0.29 |
| Sample 8 | 2324 | 3.07 | 42.36 | 46.55 | 7.76 | 0.26 |
| Sample 9 | 2325 | 3.10 | 42.30 | 46.59 | 7.69 | 0.30 |
| Sample 10 | 2326 | 3.02 | 42.37 | 46.59 | 7.75 | 0.26 |
| Sample 11 | 2327 | 3.10 | 42.29 | 46.59 | 7.75 | 0.26 |
| Sample 12 | 2328 | 3.05 | 42.33 | 46.58 | 7.78 | 0.26 |
| Sample 13 | 1084 | 2.93 | 42.54 | 46.69 | 7.10 | 0.75 |
| Sample 14 | 1085 | 2.98 | 42.48 | 46.67 | 7.17 | 0.70 |
| Sample 15 | 1086 | 2.98 | 42.60 | 46.65 | 7.21 | 0.56 |
| Sample 16 | 1087 | 3.04 | 42.58 | 46.50 | 7.18 | 0.71 |
| Sample 17 | 1088 | 2.97 | 42.52 | 46.62 | 7.33 | 0.56 |
| Sample 18 | 1089 | 2.88 | 42.51 | 46.69 | 7.34 | 0.58 |

| | | | | | |
|---|---|---|---|---|---|
| Mean | 3.04 | 42.44 | 46.58 | 7.49 | 0.46 |
| % RSD (not more than 2.0% | 3.5 | 0.3 | 0.3 | 3.9 | 44.4 |
| for Di-PEG and Tri-PEG) | | | | | |

### Example 1-C

### Purity and Characterization of an RUR_{20kD}-IL-2 Composition Via Size Exclusion High Performance Liquid Chromatography

Size exclusion high performance liquid chromatography (SEC-HPLC) can also be used to determine the purity and characterize an RUR_{20kD}-IL-2 composition using an Agilent 1200 series instrument fitted with a diode array detector (UV at 280 nm) and a Yarra SEC-2000 column (Phenomenex), and an eluent flow rate of 0.225 mL/minute. The mobile phase is 0.2M ammonium acetate (pH 5.5) at a volume ratio of 80:20 with acetonitrile. The diluent/formulation buffer contained 10 mM sodium acetate, 200 mM sodium chloride, 2% sucrose, at a pH of 5.0. Frozen RUR_{20kD}-IL-2 composition reference material and analytical samples are thawed and diluted to 1.0 mg/mL with formulation buffer. Samples are stable up to 5 days at 5° C. in solution.

Procedurally, at least one blank control of formulation buffer is first subjected to RP-HPLC via injection to ensure no interference with analysis of RUR_{20kD}-IL-2-related peaks. Next, the RUR_{20kD}-IL-2 composition, system suitability solution, is injected to ensure that aggregates or higher molecular weight species are resolved from tetra-PEG RUR_{20kD}-IL-2 fractions. RUR_{20kD}-IL-2 composition reference material or control is subsequently injected five times. RUR_{20kD}-IL-2 composition samples are next injected. RUR_{20kD}-IL-2 composition reference material/control is injected after every six sample injections and at the end of the injection sequence.

The % RSD of retention time of di-PEG and tri-PEG RUR_{20kD}-IL-2 fractions, for the first five reference material injections, is not more than 2.0%. The % RSD area percent of di-PEG and tri-PEG RUR_{20kD}-IL-2 for all reference material injections is not more than 5.0%. All RUR_{20kD}-IL-2 fraction peaks from reference and sample injections are integrated. Specifically, for a 1.0 mg/mL concentration of RUR_{20kD}-IL-2 composition, the di-PEG and tri-PEG RUR_{20kD}-IL-2 fractions above a 1.0% limit of detection (LOD) are integrated. For a 1.0 mg/mL concentration of RUR_{20kD}-IL-2, only di-PEG and tri-PEG RUR_{20kD}-IL-2 above a 3.0% LOQ were reported.

Analyses of replicate samples of RUR_{20kD}-IL-2 compositions are shown below in Tables 4 and 5, where peak areas are provided for mono-PEG, di-PEG, tri-PEG, tetra-PEG, and penta-PEG fractions of the RUR_{20kD}-IL-2 composition.

TABLE 4
% Peak areas for Mono-PEG, Di-PEG, Tri-PEG, Tetra-PEG, and Penta-PEG components of RUR_{20 kD}-IL-2 by SEC-HPLC

| | Result | RT (min) | Area % | RT (min) | Area % | RT (min) | Area % | RT (min) | Area % |
|---|---|---|---|---|---|---|---|---|---|
| Sample Name | Id | Tetra-PEG | Tetra-PEG | Tri-PEG | Tri-PEG | Di-PEG | Di-PEG | Mono-PEG | Mono-PEG |
| Sample 1 | 1119 | 21.62 | 4.59 | 22.40 | 46.73 | 23.91 | 46.99 | 28.65 | 1.70 |
| Sample 2 | 1120 | 21.62 | 4.53 | 22.41 | 46.67 | 23.91 | 47.11 | 28.66 | 1.69 |
| Sample 3 | 1121 | 21.62 | 4.45 | 22.41 | 46.75 | 23.92 | 47.04 | 28.75 | 1.76 |
| Sample 4 | 1122 | 21.63 | 4.66 | 22.42 | 46.68 | 23.92 | 46.91 | 28.76 | 1.75 |
| Sample 5 | 1123 | 21.64 | 4.63 | 22.44 | 46.81 | 23.94 | 46.80 | 28.76 | 1.77 |
| Sample 6 | 1124 | 21.64 | 4.54 | 22.43 | 46.79 | 23.94 | 46.87 | 28.86 | 1.80 |
| Mean | NA | 21.63 | 4.57 | 22.42 | 46.74 | 23.92 | 46.95 | 28.74 | 1.74 |
| % RSD | NA | 0.0 | 1.6 | 0.0 | 0.1 | 0.1 | 0.2 | 0.3 | 2.4 |
| (Acceptance | | (NA) | (NA) | (NA) | (not more | (NA) | (not more | (NA) | (NA) |
| Criteria) | | | | | than 10.0%) | | than 10.0%) | | |

TABLE 5
% Peak areas for Mono-PEG, Di-PEG, Tri-PEG, Tetra-PEG, and Penta-
PEG fractions of RUR_{20 kD}-IL-2 composition samples by SEC-HPLC

| | Result | RT (min) | Area % | RT (min) | Area % | RT (min) | Area % | RT (min) | Area % |
|---|---|---|---|---|---|---|---|---|---|
| Sample Name | Id | Tetra-PEG | Tetra-PEG | Tri-PEG | Tri-PEG | Di-PEG | Di-PEG | Mono-PEG | Mono-PEG |
| Sample 7 | 2021 | 23.32 | 4.93 | 24.12 | 45.21 | 25.58 | 46.82 | 29.39 | 3.04 |
| Sample 8 | 2022 | 23.32 | 4.83 | 24.10 | 45.67 | 25.58 | 46.97 | 29.42 | 2.52 |
| Sample 9 | 2023 | 23.32 | 4.74 | 24.11 | 45.08 | 25.59 | 46.66 | 29.44 | 3.52 |
| Sample 10 | 2024 | 23.32 | 4.90 | 24.11 | 45.05 | 25.59 | 46.74 | 29.41 | 3.31 |
| Sample 11 | 2025 | 23.31 | 4.89 | 24.11 | 45.00 | 25.58 | 46.72 | 29.41 | 3.39 |
| Sample 12 | 2026 | 23.32 | 4.86 | 24.11 | 45.12 | 25.59 | 46.90 | 29.48 | 3.12 |
| Sample 13 | 1119 | 21.62 | 4.59 | 22.40 | 46.73 | 23.91 | 46.99 | 28.65 | 1.70 |
| Sample 14 | 1120 | 21.62 | 4.53 | 22.41 | 46.67 | 23.91 | 47.11 | 28.66 | 1.69 |
| Sample 15 | 1121 | 21.62 | 4.45 | 22.41 | 46.75 | 23.92 | 47.04 | 28.75 | 1.76 |
| Sample 16 | 1122 | 21.63 | 4.66 | 22.42 | 46.68 | 23.92 | 46.91 | 28.76 | 1.75 |
| Sample 17 | 1123 | 21.64 | 4.63 | 22.44 | 46.81 | 23.94 | 46.80 | 28.76 | 1.77 |
| Sample 18 | 1124 | 21.64 | 4.54 | 22.43 | 46.79 | 23.94 | 46.87 | 28.86 | 1.80 |
| Mean | NA | 22.47 | 4.71 | 23.26 | 45.96 | 24.75 | 46.88 | 29.08 | 2.45 |
| % RSD | NA | 3.9 | 3.5 | 3.8 | 1.8 | 3.5 | 0.3 | 1.2 | 31.6 |
| (Acceptance | | (NA) | (NA) | (NA) | (not more | (NA) | (not more | (NA) | (NA) |
| Criteria) | | | | | than 10.0%) | | than 10.0%) | | |

A summary of representative analyses of different samples of an RUR_{20kD}-IL-2 composition by both RP-HPLC and SEC-HPLC is shown in Table 1. As can be seen, RUR_{20kD}-IL-2 composition preparations are demonstrate good batch-to-batch consistency with respect to the mixtures of PEGylated fractions (i.e., mono-PEGylated, di-PEGylated, tri-PEGylated, tetra-PEGylated, penta-PEGylated, etc.).

### Example 1-D

### SDS-Page

SDS-PAGE is utilized for the confirmation of an RUR_{20kD}-IL-2 composition identity. Samples of an RUR_{20kD}-IL-2 composition, a molecular weight marker, and an appropriate RUR_{20kD}-IL-2 composition reference material are loaded onto a NuPAGE Bis-Tris gel and migrated through the gel. Following electrophoresis, the gels are stained using GelCode™ Blue Safe Protein Stain. Comparison of the gel migration banding pattern to the reference material and confirmation of no new bands in the sample confirms the identity of the samples. The two most intense bands will correspond to the tri-PEGylated & the di-PEGylated fractions. The upper most band in the lanes corresponds to higher PEGylated variants and the lowest band corresponds to the mono-PEGylated variants.

### Example 1-E

### Affinity to IL-21443 Using Surface Plasmon Resonance (SPR), and Potency in U-2 OS Cells Expressing the Human IL-2Rαβα Complex

The binding affinity of an RUR_{20kD}-IL-2 composition is determined using Biacore X-100 Surface Plasmon Resonance with polarized light detection. The technique involves activating the surface of a Biacore CMS sensor chip with a 1:1 complex of N-hydroxysuccinimide 1-ethyl-3-(3-dimethylaminopropyl)-carbodiimide (NHS EDC) to generate an active NHS ester. Goat anti-human Fc antibody in sodium acetate, pH 4.0 buffer is covalently attached to the surface of the chip. Residual NHS ester is quenched with 1M ethanolamine. A 1:1 mixture of IL-2-Ra-Fc (Human IL-2Ra-Fc Chimera; Symansis) and IL-2Rβ-Fc (Human IL-2Rβ-Fc Chimera; Symansis) is captured on the chip using HBS-EP buffer (1 mM HEPES, pH 7.4, 15 mM NaCl, 0.3 mM EDTA, 0.0005% v/v surfactant P20) with 0.1% BSA. An RUR_{20kD}-IL-2 composition is serially diluted in HBS-EP buffer with 0.1% BSA and injected over the sensor chip. Kinetic binding affinities are measured by during the application of the solutions for 3 minutes (kon) followed by a 3 minute wash (koff). The ratio between koff and kon are used to calculate the kinetic binding affinity, KD. Results from triplicate analyses of two batches of an RUR_{20kD}-IL-2 composition are listed in Table 6. Binding affinities and rates are consistent for the two drug substance lots.

TABLE 6
RUR_{20kD}-IL-2 composition Binding Affinity
to IL-2Rαβ using SPR

| RUR_{20kD}-IL-2 | kₒₙ | k_{off} | K_{D} |
|---|---|---|---|
| composition Lot | (×10⁻⁴M⁻¹sec⁻¹) | (sec⁻¹) | (μM) |
| A. | 6.23, | 0.06689, | 1.07, |
| | 6.11, | 0.07009, | 1.15, |
| | 6.11 | 0.05573 | 0.912 |
| B. | 5.89, | 0.06893, | 1.17, |
| | 5.51, | 0.06674, | 1.21, |
| | 6.77 | 0.07156 | 1.06 |

Alternatively, the PathHunter® platform, a cryopreserved ready-to-use cell assay format provides a more robust and consistent cell response over that of cultured cells. An enzyme (β-galactosidase) fragment complementation assay (PathHunter® platform by DiscoverX Corporation, CA) is used to measure drug/ligand-receptor interactions. The potency of an RUR_{20kD}-IL-2 composition is measured in U-2 OS cells expressing the human IL-2Rαβα complex. The basis of the assay utilizes split enzyme fragments, which in isolation, are inactive. Two enzyme fragments are fused to the intracellular domains of either the IL-2Rβ or IL-2Rγ subunits, and upon ligand interaction with the receptor, the receptor subunits are brought into close proximity to restore enzyme activity. With the addition of a substrate, the enzyme acts and produces a luminescent signal. Receptor activation via enzyme fragment complementation is measured following incubation of sample and reference with cells for ˜6 hours. RUR_{20kD}-IL-2 compositions provide low-dose signaling through the high-affinity heterotrimeric αβγ IL-2 receptor (IL-2R).

### Example 1-F

### PEGylation Site Occupancy of an RUR_{20kD}-IL-2 Composition

The PEGylation site occupancy of RUR_{20kD}-IL-2 compositions from two lots is characterized by direct comparison of RUR_{20kD}-IL-2 composition with rhIL-2 by peptide mapping. In an RUR_{20kD}-IL-2 digest, a lysine-containing peptide may be PEGylated, and reflected by its corresponding native lysine-containing peptide having a lower abundance, as compared to the same peptide in a reference rhIL-2 digest. PEGylation site occupancy can thus be calculated based on the abundance reduction of the native peptide in the analyzed RUR_{20kD}-IL-2 digest. Furthermore, the peptide mapping of a surrogate material can be used for additional confirmation of site occupancy.

Generally, this analysis can be conducted as follows. In direct peptide mapping comparison studies, an RUR_{20kD}-IL-2 composition and an rhIL-2 reference control sample are digested simultaneously by GluC and GluC/Trypsin, followed by LC-UV/MS/MS analysis to provide peptide identification and abundance. The peptide mapping comparisons of the RUR_{20kD}-IL-2 composition and rhIL-2 are used to determine PEGylation site occupancy.

Briefly, a common peptide or peptides, without lysine, are selected as a reference or references in both RUR_{20kD}-IL-2 composition and rhIL-2 analysis. A peptide's relative intensity is the normalization to their reference(s). The relative abundance reduction of a native peptide (RR) with lysine is calculated by peptide relative intensities (Equation 1). PEGylation site occupancy at a lysine is the averaged RR from peptides containing the lysine.

$$RR ( \% Relative reduction of native peptide ) = \frac{[ \begin{matrix}( Peptide relative intensity in rhIL 2 ) - \\ ( Peptide relative intensity in RUR 20 KD - IL 2 )\end{matrix} ]}{Peptide relative intensity in rhIL 2} \qquad Equation 1$$

Peptide relative intensity (Pep/Ref)=UV Peak area (peptide)/UV Peak area (reference peptide)

The material used as the RUR_{20kD}-IL-2 composition surrogate is the product resulting from conjugation of mono-disperse 4 kD PEG to the lysines of rhIL-2. To mimic the PEGylation profile of an RUR_{20kD}-IL-2 composition, the surrogate is prepared using the same conjugation linker, and the conjugation reaction was carried out under the same reaction conditions used to prepare the RUR_{20kD}-IL-2 composition. LC MS/MS-based GluC mapping and trypsin mapping of the surrogate identify the PEGylated lysines and provide supportive information for RUR_{20kD}-IL-2.

The GluC map of RUR_{20kD}-IL-2 (a GMP lot) has 95% sequence coverage of rhIL-2. Direct comparison of the GluC map of RUR_{20kD}-IL-2 with rhIL-2 provides relative quantitation of four out of 11 lysines in RUR_{20kD}-IL-2 (See Table 7), where lysine 7 and 8 were counted as one site in the peptide map. The peptides containing the remainder of the lysines in the GluC map show evidence of the PEGylation without site differentiation. Additional Trypsin cleavage of the peptides containing lysines in the GluC/Trypsin map provides PEG occupancy at K31, K34, K42, and K47. Comparison of GluC/Trypsin mapping chromatograms from RUR_{20kD}-IL-2 and rhIL-2 show significant reductions of those peptides (See Table 7). K48 site PEGylation occupancy is not available (N/A) in the Trypsin/GluC map due to enzymatic mis-cleavage.

Peptide mapping of the 4 k PEGylated rhIL-2 surrogate identifies peptides with the 4 k PEG-labeled lysines at high mass accuracy (<5 ppm). Combining results from direct peptide mapping of RUR_{20kD}-IL-2 and the 4 k PEGylated rhIL-2 surrogate show that K7, K31 and K75 are predominant PEGylation sites (See Table 8). Less predominant PEGylation sites of RUR_{20kD}-IL-2 composition may be K8, K34, K42, K47, K53, and K63. K48 may be PEGylated, and K96 is undetermined.

PEGylation site occupancy is comparable in a second RUR_{20kD}-IL-2 composition GMP preparation, and in a development lot (See Table 7). The combined approach of GluC mapping and trypsin/GluC mapping provides lot-to-lot information for some predominant PEGylation sites of conjugates in RUR_{20kD}-IL-2 compositions.

TABLE 7
PEGylation Site Occupancy in RUR_{20 kD}-IL-
2 Compositions: a GMP lot and a DEMO Lot

| | GluC Map | | Trypsin/GluC Map | | % PEGylation | |
|---|---|---|---|---|---|---|
| Lysine | (% RR) | | (% RR) | | Site Occupancy | |
| Position | GMP Lot | DEMO Lot | GMP Lot | DEMO Lot | GMP Lot | DEMO Lot |
| K7/K8 | 56 | 53 | n\a | | 56 | 53 |
| K31 | n\a | | 53 | 56 | 53 | 56 |
| K31/34 | | | 85 | 77 | 83 | 77 |
| K42 | | | 14 | <10 | 14 | <10 |
| K47 | | | <10 | <10 | <10 | <10 |
| K48 | | | Unknown | | Unknown | |
| K53 | 24 | 16 | <10 | 10 | <10 | 13 |
| K63 | 11 | 36 | <10 | 21 | <10 | 29 |
| K75 | 53 | 65 | 45 | 47 | 49 | 56 |

| | | | |
|---|---|---|---|
| K96 | Unknown | Unknown | Unknown |

DEMO lot refers to a preparation made to demonstrate the operability of the production process.

TABLE 8
Summary of PEGylation Site Occupancy in an RUR_{20kD}-IL-
2 composition and 4k PEGylated rhIL-2 Surrogate

| Predominant | Predominant | Predominant |
|---|---|---|
| PEG sites from | PEG site from | PEG sites from |
| Direct Digestion (GluC and | Surrogate PEGylated rhIL-2 | Combined |
| GluC/Typsin map) | (GluC and Trypsin map) | Results |
| K7/K8 | K7 | K7 |
| K31 | K31 | K31 |
| K34 (possible) | K34 (not preferred) | N\A |
| K48 (unknown) | K48 | K48 (possible) |
| K75 | K75 | K75 |

### Example 1-G

### Solution Phase Stability of an RUR_{20kD}-IL-2 Composition

The stability of solutions of 1.0 mg/mL of an RUR_{20kD}-IL-2 composition (˜1 mg/mL of rhIL-2 equivalent in 10 mM sodium acetate, 200 mM sodium chloride, pH 5, containing 2% (w/v) sucrose) are evaluated under three different storage conditions—room temperature (ambient laboratory conditions), 5° C. (refrigerated), and −20° C. at 1, 3, 5, and 7-day time points by RP-HPLC as previously described.

Differences between the RUR_{20kD}-IL-2 composition solutions are evaluated against a control, freshly prepared RUR_{20kD}-IL-2 composition sample solution. Di-PEG and tri-PEG species of RUR_{20kD}-IL-2 composition samples stored at RT, 5° C., and −20° C. up to 7 days show relative differences up to 1% compared to the nominal −70° C. sample storage. The relative difference (Rel. Diff) for smaller percentage component PEGylated species of RUR_{20kD}-IL-2 (mono-PEG, tetra-PEG and penta-PEG species) is up to 8%. This indicates that solution samples stored under these representative storage conditions are stable.

### In Vitro Bioassays

In vitro methods may be used for further measurement of biological potency and biological characterization of RUR_{20kD}-IL-2 compositions, including cell-based assays to characterize bioactivity following activation of the receptor, which is representative of the IL-2 receptor complex:

### Bioassay Methodologies

| | | IL-2 | | |
|---|---|---|---|---|
| | | Receptor | Assay | |
| Bioassay | Cell Type | Form | Platform | Response |
| Cell | CTLL-2 | Murine | CellTiter- | Receptor |
| proliferation | (continuous | IL-2Rαβγ | Glo ® Cell | dimerization |
| | culture) | | Viability, | and cell |
| | | | Promega | proliferation |
| Ligand/drug | U-2 OS | Human | PathHunter ®, | Receptor |
| receptor | (frozen cells, | IL-2Rαβγ | DiscoverX | dimerization |
| interaction | ready-to-use) | | | and enzyme |
| | | | | complemen- |
| | | | | tation |
| Phospho- | CTLL-2 | Murine | Multiplex, | Receptor |
| STAT5 | (continuous | IL-2Rαβγ | Meso Scale | dimerization |
| activation | culture) | | Discovery | and phosphor- |
| | | | | ylation |

In all three assays, the data from the dose-response curve (response versus concentration) are evaluated using a non-linear regression model. The potency of the RUR_{20kD}-IL-2 composition sample is measured relative to reference material through the half-maximal effective concentration (EC₅₀) ratio.

### Example 1-H

### CTLL-2 Cell Proliferation Assay

In the cell proliferation assay, cell growth is measured in vitro using CTLL-2 cells following incubation of sample and reference for ˜26 hours where cell proliferation is measured via luminescence adenosine triphosphate-based assay (CellTiter-Glo®, Promega, WI). For example, This cell-based proliferation assay uses the CTLL-2 cell line, which exhibits a dose-dependent proliferation response to rhIL-2 protein. rhIL-2 is used as the assay control and is prepared at a different concentration range from an RUR_{20kD}-IL-2 composition in this method. This assay is performed in a 96-well plate format. CTLL-2 cells are starved of rhIL-2 in starvation media and incubated overnight for 20±3 hours in a 37° C. and 5% CO₂ incubator. Starved cells are plated in 96-well plates and a dilution series of RUR_{20kD}-IL-2 composition is fed to the cells and incubated for another 25±3 hours in a 37° C. and 5% CO₂ incubator. RUR_{20kD}-IL-2 composition induced cell growth in each well is measured using a CellTiter Glo® detection kit by Promega. CellTiter Glo® generates a luminescent signal proportional to the amount of ATP present in each well, which is directly proportional to the viable cells present. The luminescence signal is read on a SpectraMax M5 plate reader. A dose response curve of RUR_{20kD}-IL-2 composition reference material and each sample is generated by plotting luminescent signal (y-axis) to concentrations (x-axis). The plot is fitted to a 4-parameter logistic non-linear regression model. Parallel Line Analysis (PLA) software is used to assess the Equivalence Test for Difference of Slopes (parallelism), Significance of Regression, and to calculate the potency ratio of the sample in relation to the reference material in the same plate.

### Example 1-I

### Phospho-STAT5 Activation

In the phospho-STAT5 assay following receptor binding, downstream cell signaling can then activate Signal Transducer and Activator of Transcription 5 (STAT5) through phosphorylation to promote gene expression to induce cell proliferation. The activation of phospho-STAT5 is measured in CTLL-2 cells, an IL-2-dependent murine T lymphocyte cell line, using the phospho-STAT5/total STAT5 multiplexed assay (Meso Scale Discovery, MD) in response to sample and reference treatment for ˜10 minutes.

### Example 2

### In Vivo Study: Single-Dose PK/PD Study in Mice

Selective stimulation of Tregs by an RUR_{20kD}-IL-2 composition can be demonstrated in mice. C57BL/6 mice (n=4/group) are administered a single subcutaneous dose of an RUR_{20kD}-IL-2 composition at doses of 0.03, 0.1 and 0.3 mg/kg. Following administration, blood and spleen samples are collected at days 1-7 and day 10 post administration. More particularly, at each time point, blood and spleen samples are collected; samples are pooled and assessed for pharmacodynamic analysis of drug action on lymphocyte cell populations by flow cytometry (see e.g. Example 5), expressed as a fold change relative to vehicle control. In addition to changes in cell numbers, functional markers and markers of activity are quantified. Finally, plasma drug concentration is also assessed.

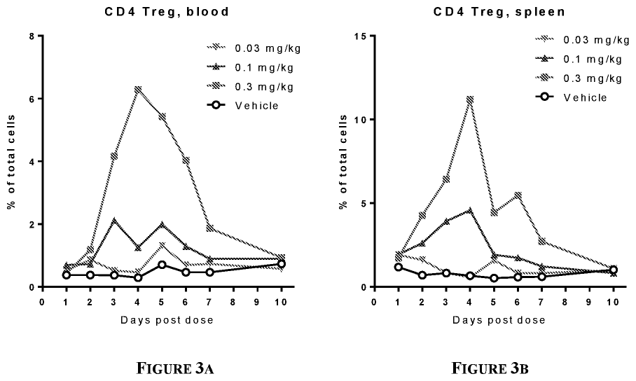

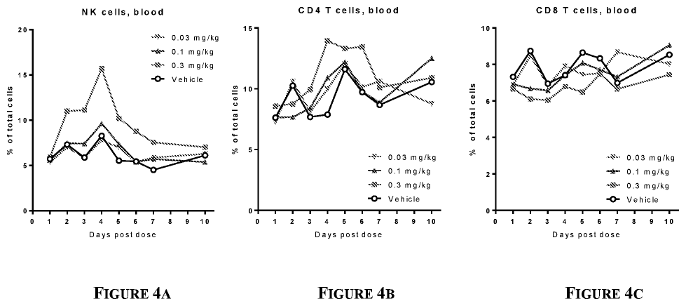

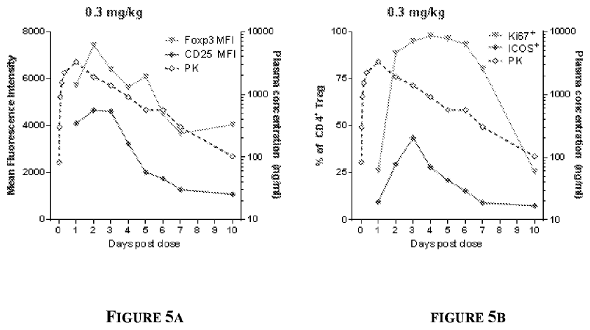

As shown in FIGS. 3A and 3B, administration of an RUR_{20kD}-IL-2 composition results in dose-dependent increases in CD4⁺ Treg in both blood and spleen, with a peak increase in cell numbers four days following administration. At the highest dose tested (0.3 mg/kg), a sustained effect on Treg mobilization is achieved, with Treg levels not returning to baseline levels until 7-10 days following administration. In blood, NK cells are elevated following administration of the highest dose tested, while changes to CD4 T cells are modest, and slight decreases in CD8 T cells occur (FIGS. 4A-C). B cells and CD8 T cells are slightly decreased following administration of the highest dose tested, a dose which also led to a less than 2-fold increase in NK cells. Markers of Treg function and activity (FIGS. 5A and 5B) demonstrate that at the highest dose tested, administration of an RUR_{20kD}-IL-2 composition leads to an increase in Treg activation, as measured by the mean fluorescence intensity (MFI) of CD25 and Foxp3. While Treg numbers do not reach maximum values until four days following administration, these activation markers achieve their maximum in the first two days following administration, slowly decreasing in accordance to the plasma exposure of RUR_{20kD}-IL-2. The percentage of rapidly proliferating Treg, as measured by Ki67, rise rapidly two days following administration and the percentage remained sustained through day 6 before returning to baseline levels. In addition, the percentage of Treg expressing the cell-surface marker inducible T cell costimulator (ICOS) is also increased, a notable finding as ICOS expression is linked to increased suppressive activity of Treg in autoimmune settings. While the increase in Ki67 and ICOS appears to be somewhat delayed relative to peak RUR_{20kD}-IL-2 composition concentration, their return to baseline levels does coincide with a decrease plasma concentration in this preclinical mouse study.

### Example 3

### In Vitro Treg Suppression Assay

The objective of this study is to assess the inhibitory function of regulatory T cells. Tregs are magnetically isolated from naive and RUR_{20kD}-IL-2 composition treated C57BL/6 mice at days 1-7 and 10 following subcutaneous administration. Treg and Tcon are co-cultured at a range of ratios from 1:2 to 1:512 for three days. Cellular proliferation is evaluated by ³H-thymidine incorporation over the final 16 hours of the assay, and the % of proliferating cells relative to plate controls is calculated.

In brief, spleens are collected from female C57BL/6 mice treated with an RUR_{20kD}-IL-2 composition at various dose levels (0.03, 0.1, and 0.3 mg/kg) or vehicle at indicated times post-dose administration (n=4 mice/treatment group/time). Single-cell isolations are prepared for each spleen, and the resultant splenocyte mixtures are pooled for each dose group at each timepoint. A portion of the pooled sample equivalent to one spleen is aliquoted for immune cell profiling. The remaining splenocyte preparation is utilized for isolation of regulatory T cells (Tregs). CD4+CD25+ Tregs are isolated from mouse spleens by magnetic-activated cell sorting (MACS) utilizing the CD4+CD25+ Regulatory T cell isolation, mouse, kit (Miltenyi Biotec, Bergisch Gladbach, Germany) according to the manufacturer's recommendations. CD4+ T cells are negatively selected and then separated into CD4+CD25− T cells and CD4+CD25+ Tregs. Naive conventional CD4+CD25− T cells (Tcon) are isolated by MACS from mouse spleens harvested from untreated animals, using the naïve CD4+ T cell isolation kit (Miltenyi Biotec) and following the manufacturer's recommended procedure.

In vitro suppression assays are carried out in RPMI 1640 medium supplemented with 10% fetal bovine serum, 2 mM L-glutamine, 1 mM sodium pyruvate, 0.5 μM β-mercaptoethanol, and 1× antibiotic/antimycotic (100 units/mL penicillin, 100 μg/mL streptomycin and 250 ng/mL amphotericin B). 5×10⁴ Tcon are stimulated with beads coated with anti-CD3 and anti-CD28 (T Cell Activation/Expansion kit, mouse, Miltenyi Biotec) at a ratio of 2 beads to each Tcon in 100 μL culture medium in 96-well round-bottom plates. The suppressive capacity of Tregs is assessed by the addition of Tregs to Tcon at different ratios (Treg:Tcon ratios of 2:1 to 1:512). Each Treg:Tcon ratio is tested in triplicate. Cells are co-cultured for 72 hours at 37° C. and 5% CO₂ in a humidified atmosphere; 16 hours prior to the termination of the assay, 0.5 μCi [3H]-thymidine is added to wells. After washing cells free from unincorporated [3H]-thymidine, thymidine uptake is measured as counts-per-minute (CPM) using a microplate scintillation counter (TopCount NXT, Perkin Elmer). Individual CPM values are normalized to maximal proliferation by dividing by the mean CPM recorded for the four lowest Treg:Tcon dilutions. Concentration-response curves are graphed using four-parameter non-linear regression and 1/y2 weighting in Prism® 6.03 (GraphPad Software, San Diego, Calif.).

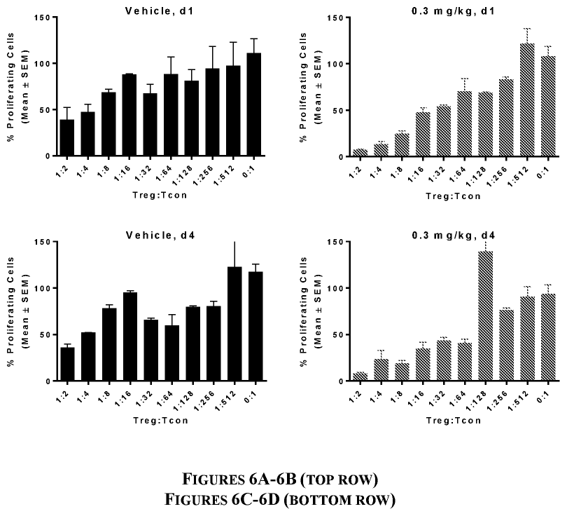

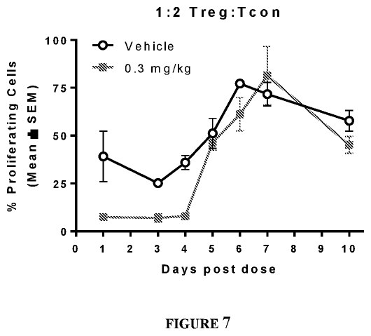

As shown in FIGS. 6A-D, splenic Treg's isolated from vehicle treated mice at 1 and 4 days following the study initiation exhibit suppressive capacity with the greatest suppression occurring at a ratio of 1:2. However, Treg's isolated at these time points following administration of an RUR_{20kD}-IL-2 composition exhibit a greatly increased suppressive capacity, as evidenced by decreased Tcon proliferation, particularly at ratios greater than 1:8. The relative suppressive capacity of isolated Treg's cultured with Tcon at a ratio of 1:2 is also assessed over time (FIG. 7). Following RUR_{20kD}-IL-2 administration, increased Treg suppressive activity is maintained for four days before returning to baseline activity exhibited by the vehicle control-treated group.

### Example 4

### Evaluation of an RUR_{20kD}-IL-2 Composition in a Mouse KLH DTH Efficacy Model

To assess the ability of Treg induction by administration of an RUR_{20kD}-IL-2 composition to suppress T-cell antigen-driven inflammation, Balb/c mice (n=6-10/group) are utilized in a model of delayed-type hypersensitivity (DTH). Mice are sensitized subcutaneously in their dorsal area with a 100 μl subcutaneous injection containing 100 μg keyhole limpit hemocyanin (KLH) in an emulsion containing Complete Freund's Adjuvant (CFA) and Incomplete Freund's Adjuvant, at a ratio of 1:1:1 respectively. Five days later, baseline ear thickness is measured prior to challenge with 10 μg of KLH intradermally in the left ear, with the right ear remaining untreated. Ear thickness measurements are measured with calipers at 24, 48, 72 and 96 hours post KLH challenge in all groups. RUR_{20kD}-IL-2 composition is administered on day 0, at the time of sensitization, with subcutaneous doses ranging from 0.003 mg/kg to 0.3 mg/kg every three days. A positive control consisting of cyclosporin (10 mg/kg, single dose) was administered on day 0.

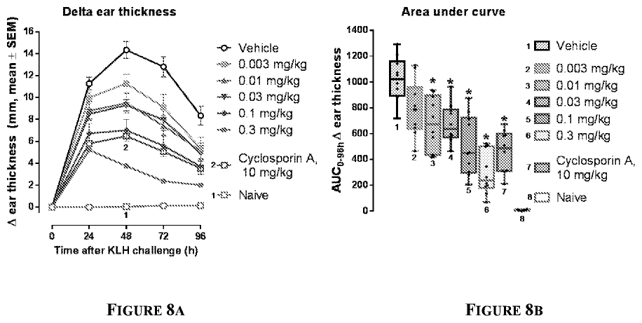

As shown in FIGS. 8A, 8B, following the antigen challenge, ear swelling is induced with a mean increase in ear thickness reaching a maximum of over 14 mm at 48 hours. Naive, non-challenged ears exhibited no changes in thickness during the course of study. The administration of an RUR_{20kD}-IL-2 composition through the sensitization and challenge period in this study leads to significant dose-dependent decreases in ear swelling as evidenced by reduced inflammation at each time point relative to the vehicle control. To more quantitatively assess the effect following challenge, an AUC of the change in thickness is calculated for each treatment group (AUC₀₋₉₆ₕ). As shown in FIGS. 8A and 8B, the minimally effective dose is 0.01 mg/kg q3d, while the maximal effect is achieved with 0.3 mg/kg q3d. AUC values statistically significant from the vehicle group are noted with an asterisk (p<0.05; ANOVA, Tukey's). Taken together, this data demonstrates that the enhanced mobilization and activation of Treg achieved after administration can suppress antigen-driven inflammatory mechanisms in vivo.

The activity of an RUR_{20kD}-IL-2 composition of Example 1 is assessed after in vivo administration in rodent and cynomologous monkey. In mice, an RUR_{20kD}-IL-2 composition leads to dose-dependent increases in Treg which reach a maximum four days after administration. Flow cytometric analysis of Treg induced by an RUR_{20kD}-IL-2 composition in mice showed that markers of Treg activation such as Foxp3 and CD25 mean fluorescence intensity (MFI) reach their maximum value within the first two days following administration, and gradually decreased over time in accordance with plasma exposure of the RUR_{20kD}-IL-2 composition. The percentage of Treg actively proliferating also achieves its maximum value within two days following administration and is sustained through day 6. Expression of the Treg functional marker ICOS peaks at day 3 before returning to baseline by day 7. Treg isolated from the spleens of treated mice greatly increase their suppressive capacity in the first four days following administration before returning to basal levels of activity. An RUR_{20kD}-IL-2 composition of Example 1 suppressed an antigen-driven inflammatory reaction in a delayed-type hypersensitivity (DTH) mouse model when administered every three days.

### Example 5

### Single-Dose Study in Cynomologous Monkey

In this study, cynomologous monkey, one female and one male, are administered 25 μg/kg of an RUR_{20kD}-IL-2 composition subcutaneously. A series of blood samples are taken from each animal before treatment (day −6 and −1) and at multiple intervals following treatment for assessment by flow cytometry of Treg cell numbers and activation state.

For immunophenotyping analysis, blood samples (approximately 1.0 mL) are collected from each monkey at the following time points: Pre-treatment (Day −6 and −1), Day 2, Day 3, Day 4, Day 5, Day 6, Day 7, Day 10, Day 14, and Day 21 post treatment. Venipuncture samples are collected into tubes containing the anticoagulant, K₂EDTA. Tubes are placed on wet ice pending transfer. The whole blood samples are analyzed by flow cytometry using the following panels, and the samples are analyzed for the following:
T cell panel: CD45/CD3/CD4/CD8/ICOS
TB/NK panel: CD45/CD3/CD16/CD20
pSTAT5 panel: CD3/CD4/CD8/CD25/CD127/pSTAT5
Treg panel 1: CD3/CD4/CD8/CD25/FoxP3/Ki67
Treg panel 2: CD3/CD4/CD8/CD25/FoxP3/Helios
   Computerized systems can be used for the conduct of the study, for example flow cytometry data acquisition can use BD FACSCanto II/FACSDiva LEGENDPlex Data Analysis Software, and flow cytometry data analysis can use De Novo FCS Express software.

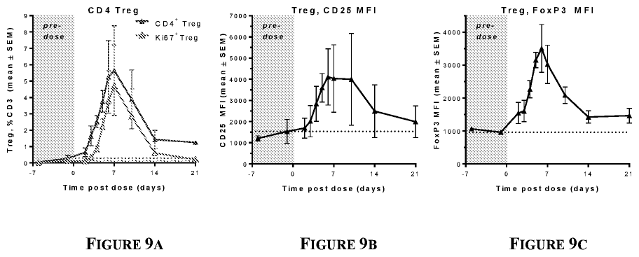

Values from male and female are averaged, and the magnitude of change is shown relative to d-1 values, marked by the dotted line. As shown in FIG. 9, Treg cell numbers rise substantially following administration, reaching their maximum level seven days following administration and returning to near d-1 levels by days 14-21. As shown by the open triangles in FIG. 9A, nearly all Treg's induced by an RUR_{20kD}-IL-2 composition are proliferative, as measured by Ki67.

The relative activation state of Treg's stimulated by administration of an RUR_{20kD}-IL-2 composition is further measured by the mean fluorescence intensity (MFI) of FoxP3 and CD25. CD25 MFI reaches its maximum value at day 6 and then plateaus through day 10 before returning to near pre-dose levels by day 21. FoxP3 MFI also reaches a maximum 6 days following administration before nearly returning to pre-dose levels at day 14-21. Taken together, these data demonstrate the translatability of the findings in mice to cynomologous monkey, as a similar magnitude of Treg induction in blood is seen, which is accompanied by an increased Treg activation. However, in contrast to the findings in mice, the effects in cynomolgus monkeys are more prolonged in nature.

### Example 6

### Single Dose Pharmacokinetics and Toxicokinetics in Mice, Rats, and Monkeys

Results of the single dose pharmacokinetics/toxicokinetics of an RUR_{20kD}-IL-2 composition in mice, rats, and monkeys are summarized. Details of the dosage regimen are provided in Table 10.

TABLE 10
Overview of RUR_{20 kD}-IL-2 Composition Single-Dose
Pharmacokinetic and Toxicokinetics Studies

| | | | Dose | Dose | Dose | |
|---|---|---|---|---|---|---|
| | Route of | | Volume | Concentration | Level | Matrices |
| Species | Administration | Gender | (mL/kg) | (mg/mL) | (mg/kg) | collected |
| Mouse | Subcutaneous | Female | 5 | 0.006, 0.02, | 0.03, 0.1, | Plasma |
| (Pharmacokinetics) | | | | 0.06 | 0.3 | |
| Rat | Intraveneous | Male | 1 | 0.01, 0.1, | 0.01, 0.1, | Plasma, |
| (Pharmacokinetics) | | | | 1 | 1 | Urine |
| | Subcutaneous | Male | 1 | 0.01, 0.1, | 0.01, 0.1, | Plasma |
| | | | | 1 | 1 | |
| Rat | Subcutaneous | Female | 0.2, 1.5 | 0.05, 0.5, | 0.01, 0.1, | Plasma |
| (Toxicokinetics) | | | | 1 | 1.5 | |
| Monkey | Intraveneous | Male | 0.2 | 0.125 | 0.025 | Plasma, |
| (Pharmacokinetics) | | | | | | Urine |
| | Intraveneous | Female | 0.2 | 0.125 | 0.025 | Plasma, |
| | | | | | | Urine |
| | Subcutaneous | Male | 0.2 | 0.125 | 0.025 | Plasma |
| | Subcutaneous | Female | 0.2 | 0.125 | 0.025 | Plasma |

For the mouse study, vehicle for the RUR_{20kD}-IL-2 composition is 10 mM sodium acetate, 200 mM sodium chloride and 2% sucrose (pH5). For the rat and monkey studies, vehicle for the RUR_{20kD}-IL-2 composition is 50 mM sodium acetate, 200 mM sodium chloride and 2% sucrose (pH 5).

Following subcutaneous administration, the RUR_{20kD}-IL-2 composition is slowly absorbed with Tₘₐₓ of 0.33-1.0, 1.0-2.3, and 2.0 days in mice, rats, and monkeys, respectively (Table 11). RUR_{20kD}-IL-2 composition plasma exposures increase more or less dose proportionally in mice and rats. Bioavailability is in the range of 29.8-46.0% in rats and 86.2% in monkeys.

The volume of distribution at steady state (Vₛₛ) of the RUR_{20kD}-IL-2 composition appears to increase in the rat with dose and ranged between 25.1 (0.01 mg/kg) and 52.6 mL/kg 1.0 mg/kg) (Table 12). Overall, Vₛₛ is 1-2 fold and 2-4 fold greater than species-specific plasma volume in rats and monkeys, respectively, suggesting that RUR_{20kD}-IL-2 stays mostly in the vascular space.

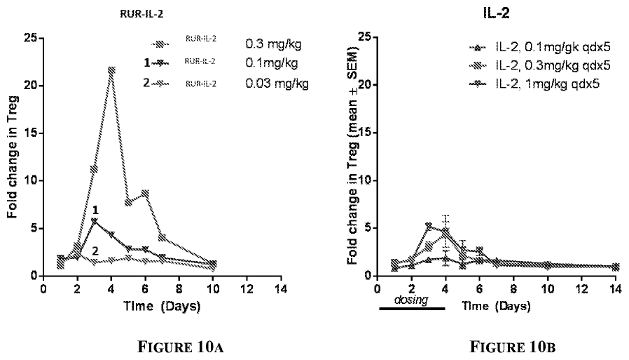

Plasma clearance (CL) is very low (0.560-1.14 mL/hr/kg in rats and 0.245 mL/hr/kg in monkeys) (Table). Following intravenous or subcutaneous dosing, the RUR_{20kD}-IL-2 composition concentrations appear to exhibit a mono-exponential decay with half-lives of 1.85-2.24 days in mice, 1.25-2.44 days in rats, and 10.4-12.9 days in monkeys (Tables 11 and 12 and FIGS. 10A, 10B). Renal excretion of RUR_{20kD}-IL-2 is projected to be low due to its average molecular weight of 63 kDa which is near the molecular weight cut-off for the glomerulus filter.

TABLE 11
Mean ± SE Plasma Pharmacokinetic/Toxicokinetic Parameters after Administration of a Single Subcutaneous
Dose of an RUR_{20 kD}-IL-2 Composition to C57BL/6 Mice, Sprague-Dawley Rats, or Cynomolgus Monkeys

| Species | SC | | | | | | | Absolute Bio- |
|---|---|---|---|---|---|---|---|---|
| (Study | Dose | AUCinf | AUClast | Cmax | Half-life | MRT_{inf} | Tₘₐₓ | availability |
| number) | (mg/kg) | (hr*μg/mL) | (hr* μg/mL) | (μg/mL) | (day) | (day) | (day) | (%) |
| Mouse | 0.03 | 22.9 | 21.5 | 0.333 | 2.24 | 3.46 | 0.33 | NA |
| (LS-2016- | 0.1 | 102 | 97.4 | 1.14 | 2.24 | 3.39 | 0.33 | NA |
| 2057, LS- | 0.3 | 245 | 238 | 3.33 | 1.85 | 2.98 | 1.00 | NA |
| 2016-2073) | | | | | | | | |
| Rat | 0.01 | 7.16 ± 0.53 | 7.12 ± 0.51 | 0.095 ± 0.006 | 1.35 ± 0.06 | 2.70 ± 0.06 | 1.33 ± 0.33 | 40.2 |
| (LS-2016- | 0.1 | 35.7 ± 5.10 | 35.5 ± 5.10 | 0.38 ± 0.04 | 1.92 ± 0.08 | 3.42 ± 0.28 | 2.0 ± 0.00 | 29.8 |
| 2033) | 1 | 404 ± 79.0 | 398 ± 74.0 | 4.86 ± 0.93 | 1.27 ± 0.32 | 2.82 ± 0.16 | 2.30 ± 0.33 | 46.0 |
| Rat¹ | 0.01 | 5.23 | 5.21 | 0.06 | 1.41 | 2.63 | 1 | NA |
| (LS-2016- | 0.1 | 73.0 | 71.1 | 0.64 | 2.44 | 4.08 | 1 | NA |
| 004) | 1.5 | 678 | 672 | 9.30 | 1.97 | 3.07 | 2 | NA |
| Monkey² | 0.025 | 86.0 | 65.4 | 0.25 | 10.4 | 15.0 | 2.0 | 86.2 |
| (LS-2016- | | | | | | | | |
| 2026) | | | | | | | | |

AUCinf: Area under the plasma concentration-time curve from time zero to infinite time;
AUClast: Area under the plasma concentration-time curve from time zero to the last measurable concentration; C max: Maximum observed plasma concentration; MRTinf: Mean residence time;
T max: Time of observed maximum plasma concentration

1. PK parameters are based on mean value of three rats per time point.

2. Mean of male and female monkey.

TABLE 12
Mean ± SE Plasma Pharmacokinetic Parameters after Administration of a Single Intravenous
Dose of an RUR_{20 kD}-IL-2 Composition to Sprague-Dawley Rats or Cynomolgus Monkeys

| | IV Dose | AUCinf | AUClast | CL | Half-life | MRTinf | Vss |
|---|---|---|---|---|---|---|---|
| Species | (mg/kg) | (hr* μg/mL) | (hr* μg/mL) | (mL/hr/kg) | (day) | (day) | (mL/kg) |
| Mouse | ND | ND | ND | ND | ND | ND | ND |
| Rat | 0.01 | 17.8 ± 1.00 | 17.8 ± 0.90 | 0.56 ± 0.03 | 1.25 ± 0.16 | 1.87 ± 0.21 | 25.1 ± 1.80 |
| | 0.1 | 120 ± 8.00 | 120 ± 8.00 | 0.84 ± 0.06 | 1.67 ± 0.01 | 2.18 ± 0.04 | 44.1 ± 3.50 |
| | 1 | 877 ± 39.0 | 870 ± 35 | 1.14 ± 0.05 | 1.56 ± 0.22 | 1.90 ± 0.16 | 52.6 ± 7.00 |
| | | 39100 | | | | | |
| Monkey* | 0.025 | 100 | 71.2 | 0.25 | 12.9 | 16.8 | 100 |

ND: Not determined; AUCinf: Area under the plasma concentration-time curve from time zero to infinite time; AUClast: Area under the plasma concentration-time curve from time zero to the last measurable concentration; CL: Clearance; MRTinf: Mean residence time; Vss: Apparent volume of distribution at steady-state.
*Mean of male and female monkey.

### Example 7

### Comparative Study in Mice

A study essentially similar to that described in Example 2 is conducted in which C57BL/6 mice are administered either a single subcutaneous dose of an RUR_{20kD}-IL-2 composition at 0.03, 0.1 and 0.3 mg/kg or are administered unmodified IL-2 (aldesleukin) at dosages of 0.03 mg/kg (qdd×5), 0.1 mg/kg (qd×5) and 1 mg/kg (qd×5). Following administration, blood and spleen samples are collected and analyzed for pharmacodynamic analysis of drug action on lymphocyte cell populations by flow cytometry, expressed as a fold change relative to vehicle control. Results are shown in FIGS. 10A and 10B (RUR_{20kD}-IL-2 composition is labelled “RUR-IL-2”, Aldesleukin is labelled “IL-2”).

### Example 8

### Investigation of the Efficacy of an RUR_{20kD}-IL-2 Composition in a Mouse Model of Systemic Lupus Erythematosus (SLE)

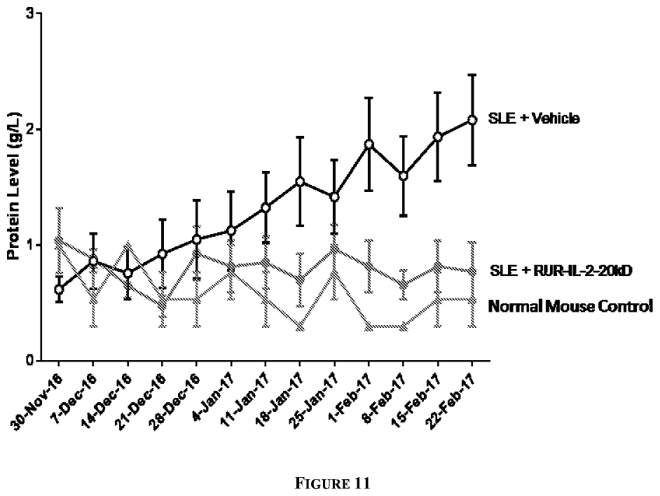

This study is conducted to determine the efficacy of an RUR_{20kD}-IL-2 composition on the development and progression of SLE and its associated characteristics using a MRL/MpJ-Faslpr mouse model, the most commonly studied mouse model of this disease (Perry, D., et al., J Biomed Biotechnol 2011:271694). The MRL/MpJ-Faslpr mouse model develops an autoimmune disease that reflects pathologies of human SLE, including lymph node enlargement, increased IgG levels, antinuclear antibody production, proteinuria, and kidney failure caused by inflammation of the glomeruli. A stock solution of an RUR_{20kD}-IL-2 composition as described in Example 1 is used as the test article (1.58 mg/mL) supplied in vehicle (transparent liquid; 10 mM sodium acetate/200 mM sodium chloride/2% (w/v) sucrose), prepared in sterile water for injection (SWFI), USP; pH 5.0±0.1). On dosing days, a suitable quantity of test article is withdrawn and diluted with vehicle to arrive at the desired dosing concentration (0.03 mg/kg dose and 0.3 mg/kg dose); dose volume was 5 mL/kg. Animals used for the study are MRL/MpJ-Faslpr mice and MRL/MpJ naive, female mice, aged from 6-8 weeks. Animals are assigned to treatment groups by randomization. Treatment groups are described in Table 13 below. 45 MRL/MpJ-Faslpr mice are randomized into 3 groups (15 each for Groups 2-4) based on body weight and level of protein content in urine before the commencement of the experiment. Animals in Groups 2-4 receive vehicle or test article delivered subcutaneously as described in Table 6. Group 1-MRL/MpJ mice receive the vehicle as a negative control. Three (3) days after the first dose administration on Week 8, 3 mice from Groups 2-4 are humanely sacrificed and blood samples were collected and processed. Body weights are measured twice a week from the commencement of the study and continued throughout. Skin lesion pictures were taken when first observed and then at one week intervals. Urine is obtained the day before dosing (at baseline) and then collected weekly thereafter. Protein levels in the urine are measured using a Siemens Clinitek Status Analyzer. On sacrifice day (3 days after the last dose at the end of Week 20), all mice are anesthetized by intraperitoneal injection of chloral hydrate (50 mg/kg). Blood samples are collected, and centrifuged at 10000 r/min for 10 min to obtain serum samples. The serum is stored at −80° C. until clinical biochemistry testing. Serum samples (100 μl) are analyzed for anti-dsDNA level by ELISA (Mouse Anti-dsDNA IgG-specific ELISA Kit, Alpha Diagnostic International, Cat. No. 5120) and the serum tested for BUN concentration using a Hitachi 7020 Automatic Biochemistry Analyzer. For lymphocyte analysis, blood samples are collected in EDTA-K tubes and tested for CD3/CD4/CD8/Treg/NK/B cell % by flow cytometry. Results are shown in FIG. 11. As shown therein, administration of an RUR_{20kD}-IL-2 composition at a dose of 0.3 mg/kg is effective to suppress the biomarker of kidney damage (i.e., protein levels in urine) to nearly the same levels as observed in normal mice. This study further elucidates the effect of RUR-IL-2-induced Tregs on control of the physiological immune response and disease progression in a representative animal model of SLE.

TABLE 13
Treatment Groups

| | | | Concentration | Dosage | | | |
|---|---|---|---|---|---|---|---|
| Group | Test Article | N | (mg/mL) | (mL/kg) | (mL/kg) | Route | Regimen |
| 1 | Vehicle^{a} | 3 | N/A | 5 | N/A | s.c. | Twice weekly, |
| | (MRL/MpJ) | | | | | | from week 8 |
| | | | | | | | to week 20 |
| 2 | Vehicle^{a} | 15 | N/A | 5 | N/A | s.c. | Twice weekly, |
| | (MRL/MpJ-Faslpr) | | | | | | from week 8 |
| | | | | | | | to week 20 |
| 3 | RUR_{20 kD}-IL-2 Dose 1 | 15 | 0.006 | 5 | 0.03 | s.c. | Twice weekly, |
| | (MRL/MpJ-Faslpr) | | | | | | from week 8 |
| | | | | | | | to week 20 |
| 4 | RUR_{20 kD}-IL-2 Dose 2 | 15 | 0.06 | 5 | 0.3 | s.c. | Twice weekly, |
| | (MRL/MpJ-Faslpr) | | | | | | from week 8 |
| | | | | | | | to week 20 |

^{a}vehicle of test article

### Example 9

### Study of an RUR_{20kD}-IL-2 Composition in an Antigen Dependent, T Cell-Mediated, Delayed-Type Hypersensitivity (DTH) Model

This study models how in vivo Treg stimulation and expansion, by an RUR_{20kD}-IL-2 composition, can downregulate T cell-mediated delayed-type hypersensitivity (DTH) response in an antigen dependent manner, in a food allergy model where a high degree of anaphylaxis is established.

To develop the DTH model, Balb/c mice are sensitized with a subcutaneous administration of a model antigen keyhole limpet hemocyanin (KLH) emulsified in complete and incomplete Freund's adjuvant. Subcutaneous administration of an RUR_{20kD}-IL-2 composition (0.003, 0.01, 0.3, 0.1 or 0.3 mg/kg, q3d) or Cyclosporin A (10 mg/kg, qd) is initiated on day 0 and continued through day 8, with an intradermal challenge of KLH administered on day 5, and ear swelling measured for four days. Inflamed ears are subjected to immunohistochemistry (IHC) to quantify percent of FoxP3+ Treg cells post KLH challenge. The specificity of response is assessed after an additional 3-4 weeks with no treatment by either KLH rechallenge or conducting sensitization and challenge with an unrelated antigen ovalbumin (OVA). To understand the effect of RUR_{20kD}-IL-2 composition-expanded Tregs on food allergen, Balb/C mice are sensitized twice in a week by emulsifying OVA with alum intraperitoneally. Post ten days of 2^{nd} sensitization, mice are challenged with OVA eight times orally, every alternative day. Subcutaneous administration of an RUR_{20kD}-IL-2 composition (0.1 mg/kg, q3d×3) or Cyclosporin A (10 mg/kg, qd) is initiated on day 0 and continued through day 8. Severity of allergic response is assessed by clinical scoring within 30-45 min of post 8^{th} challenge. Further, serum mast cell protease 1 (MCPT 1) and OVA specific IgE titers are quantified. The percent Treg is determined by flow cytometry in peripheral blood and in spleen.

In this mouse model of DTH, RUR_{20kD}-IL-2 composition administration suppressed the inflammatory response to KLH rechallenge in a dose dependent manner. IHC analysis of inflamed ears show significant infiltration of FoxP3+ Treg cells. The suppressive effect on inflammation is durable and antigen-specific as exemplified by re-challenge post 3-4 weeks with same antigen and unrelated antigen post sensitization with no further RUR_{20kD}-IL-2 composition administration. Finally, administration of RUR_{20kD}-IL-2 composition is found to be efficacious in decreasing the high degree of anaphylaxis symptoms caused by repeated administration of model food allergen, OVA. The decrease in clinical scores of anaphylaxis is correlated with a significant decrease in levels of MCPT1 and anti-OVA specific IgE titers as well as a significant increase in Tregs. RUR_{20kD}-IL-2 composition demonstrated antigen specific and durable Treg expansion and therapeutic responses in this KLH hypersensitivity model of mice. Further, An RUR_{20kD}-IL-2 composition is found to be efficacious in a food allergy model. This data supports use of RUR_{20kD}-IL-2 compositions for antigen specific inflammation as may be the case in autoimmune and/or inflammatory diseases.

Preclinical evidence provided herein support the concept that IL-2 conjugate Treg stimulator RUR_{20kD}-IL-2 compositions increase number and suppression function of regulatory T cells for the treatment of autoimmune and inflammatory disorders. Impaired IL-2 production and regulatory T cell dysfunctions have been implicated as an immunological mechanism in multiple autoimmune diseases. While low-dose IL-2 can be used to stimulate Tregs for clinical benefit, poor pharmacokinetics necessitates daily delivery, adverse events are dose-limiting, and Treg increases are modest and short-lived. RUR_{20kD}-IL-2 compositions provide an IL-2 conjugate Treg stimulator intended for low dose subcutaneous administration to selectively restore Treg homeostasis with minimal impact on conventional T cell function. Herein is provided data to characterize the ability of RUR_{20kD}-IL-2 compositions to selectively expand the numbers and activity of Tregs in mouse and non-human primate models and to assess the efficacy of RUR_{20kD}-IL-2 compositions in models of autoimmunity. The affinity to the IL-2 receptor is assessed by surface plasmon resonance. Activity in human PBMC can be measured by pSTAT5 induction in multiple lymphocyte populations using flow cytometry and time-of-flight mass cytometry (CyToF). In vivo activity after subcutaneous administration in C57BL/6 mice or cynomolgus monkey is measured by changes in lymphocyte numbers and activation by flow cytometry. Ex vivo Treg function is determined by the inhibition of Tcon proliferation by isolated splenic Treg. Efficacy is assessed in a model of systemic lupus erythematosus (SLE) using MRL/MpJ-Faslpr mice. RUR_{20kD}-IL-2 compositions have greatly attenuated affinity for human IL-2Rβ relative to IL-2Ra and IL-2Rαβ complexes, suggesting preferential activation of Tregs that express the high affinity IL-2Rαβγ over Tcon, which express the low-affinity IL-2Rβγ. In vitro, Tregs are more sensitive to RUR_{20kD}-IL-2 composition stimulation, showing increased STAT5 phosphorylation relative to other lymphocyte subsets in human PBMC. In mice, a single administration leads to sustained Treg mobilization for 7-10 days in blood and spleen without Tcon activation, an effect concomitant with induction of Treg activation markers and increased ex vivo suppressive capacity. In cynomolgus monkey, plasma exposure is more prolonged with sustained Treg mobilization and activity for over 14 days after a single administration—a response superior in magnitude, duration and specificity compared to an equivalent total dose of rhIL-2 administered daily for five days. Finally, an RUR_{20kD}-IL-2 composition is efficacious in mouse models of SLE. In the SLE model, repeat administration of an RUR_{20kD}-IL-2 composition over 12 weeks sustains Treg elevation, reduces blood urea nitrogen and returns urine protein levels and kidney histopathology to normal. In a cGVHD model, repeat administration of an RUR_{20kD}-IL-2 composition increases Tregs and decreases germinal center B cells in spleen, and reverses lung dysfunction. RUR_{20kD}-IL-2 compositions delivers sustained, preferential activation of Tregs and demonstrates efficacy in model systems of SLE.

### Example 10

### A Phase I, Double-Blind, Randomized Placebo-Controlled Study to Evaluate the Safety, Tolerability, Pharmacokinetics, and Pharmacodynamics of a Single Ascending Subcutaneous Dose of an RUR_{20kD}-IL-2 Composition in Healthy Volunteers

A double-blind, randomized, placebo-controlled study is conducted to evaluate the safety, tolerability, pharmacokinetics, and pharmacodynamics of single ascending low sub-cutaneous doses of an RUR_{20kD}-IL-2 composition (RUR_{20kD}-IL-2) in healthy volunteers. The study is divided into seven cohorts, in which subjects received 0.3, 1.0, 3.0, 6.0, 9.0, 13.5 or 20.0 μg/kg RUR_{20kD}-IL-2. Twelve subjects are randomized to each dose cohort, nine of whom received a single subcutaneous dose of RUR_{20kD}-IL-2 while three received placebo. RUR_{20kD}-IL-2 is formulated as a sterile liquid for subcutaneous injection that was diluted with sterile 0.9% sodium chloride solution. The drug product is supplied in single-use glass vials and stored at 2-8° C. Each vial of the drug product contained 0.75±0.1 mg of rhIL-2 (based upon RUR_{20kD}-IL-2). RUR_{20kD}-IL-2 is formulated in 10 mM sodium acetate, 150 mM sodium chloride, 2% (w/v) sucrose, pH 5.0 at a concentration of approximately 1.0 mg/mL protein. Placebo is a commercially available 0.9% sodium chloride solution. A starting dose of 0.3 μg/kg is chosen using the minimally anticipated biological effect level (MABEL) approach and is supported by the no observed adverse effect level (NOAEL) in the most sensitive species from nonclinical toxicology studies. The starting dose is set at 0.3 μg/kg to allow for evaluation of RUR_{20kD}-IL-2 pharmacokinetics and safety. Two subjects, one receiving RUR_{20kD}-IL-2 and one placebo, are dosed in a double-blind manner and monitored for possible side-effects for a period of at least 7 days prior to initiation of the study.

The primary objective of the study is to evaluate the safety and tolerability of RUR_{20kD}-IL-2 administered as a single subcutaneous dose. The secondary objectives of the study are to (1) observe the time course and extent of changes in the number and/or activity of regulatory T cells (Tregs), (2) characterize the pharmacokinetic (PK) profile of RUR_{20kD}-IL-2 administered as a single subcutaneous dose, and (3) assess the immunologic effects of RUR_{20kD}-IL-2 in blood, including effects on cytokines, T cells, other peripheral blood populations, other serum proteins, changes in gene expression, and anti-drug antibodies. In a first phase of the study, immune markers are tested pre-dose up to 20 hours post-dose. Specifically, Tregs, CD4⁺-T cells, CD8⁺-T cells, natural killer (NK) cells, cytokines, soluble CD25, and RNA are tested in RUR_{20kD}-IL-2- and placebo-receiving cohorts. In subsequent phases, the same immune markers are also tested at 4-, 5-, 6-, 7-, 8-, 10-, 12-, 15-, 18-, 20-, 25-, 30-, 40, and 50-days post-dose.

No dose-limiting toxicities (DLTs), serious adverse events (SAEs), deaths, or clinically significant abnormalities are reported. Adverse events (AEs) are limited to mild (Grade 1) injection site reactions, and no evidence was observed of AEs known to be associated with high dose IL-2.

Preliminary PK analysis shows that RUR_{20kD}-IL-2 reached maximum concentrations around 4-6 days post-dose in most subjects, with little change in concentrations up to approximately 2 weeks post-dose, after which concentrations declined with a half-life of approximately 8-9 days.

Pharmacodynamic (PD) assessment reveals that RUR_{20kD}-IL-2 leads to a dose-dependent increase in circulating CD4+FoxP3+CD25^{bright} Tregs. In the 3.0, 6.0, 9.0, 13.5, and 20.0 μg/kg single-dose cohorts, there is a sustained increase in the absolute numbers of circulating CD4+FoxP3+CD25^{bright} Tregs, with levels not returning to baseline until approximately 20 to 25 days following administration. There is a mean increase in the numbers of CD4+FoxP3+CD25^{bright} Tregs of 3-, 3.5-, 4.1-, 5-fold, and 8.1-fold, compared to pre-dose at the 3.0, 6.0, 9.0, 13.5, and 20.0 μg/kg doses, respectively. There is also an increase in the total CD4+FoxP3+CD25+Treg population at 3.0, 6.0, 9.0, 13.5, and 20.0 μg/kg doses, but the magnitude of the change is smaller than observed for the CD4+FoxP3+CD25^{bright} Tregs. There is no change in the numbers of Tregs in the RUR_{20kD}-IL-2-treated subjects versus placebo subjects at 0.3 and 1.0 μg/kg doses compared with those receiving placebo. The primary effect of RUR_{20kD}-IL-2 is seen on Tregs, as no changes in percentage or numbers of T cell populations (CD4+, CD8+) are observed with RUR_{20kD}-IL-2 at any dose. There is a small increase in the percentage and absolute numbers of NK cells at 13.5 and 20.0 μg/kg without evidence of AEs associated with high-dose IL-2.

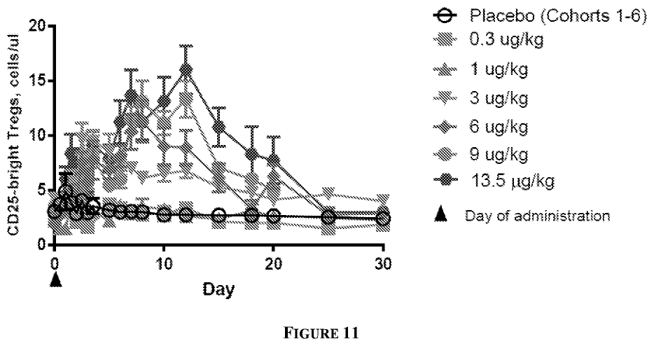

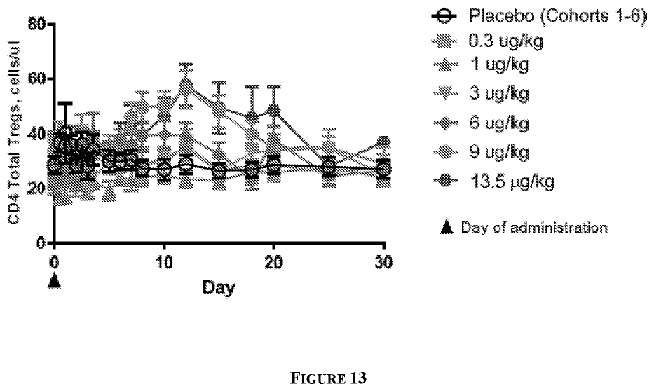

As shown in FIG. 12, an RUR_{20kD}-IL-2 composition led to a dose-dependent increase in CD4+FoxP3+CD25bright Tregs. At 3.0, 6.0, 9.0, and 13.5 μg/kg, there was a sustained increase in the absolute numbers of CD4+FoxP3+CD25bright Tregs, with levels not returning to baseline until 20-25 days following administration. There was a mean increase in the numbers of CD4+FoxP3+CD25bright Tregs of 3.0-fold, 3.5-fold, 4.1-fold, and 5.0-fold compared to placebo at 3.0, 6.0, 9.0, and 13.5 μg/kg dose respectively, with a maximal response shifting from a peak at 84 hours at 3.0 μg/kg to a more extended peak response lasting from 7 to 12 days at 13.5 μg/kg, before returning to baseline levels by Day 20-25. As shown in FIG. 13, there also was a dose-dependent increase in the total CD4+FoxP3+CD25+ Treg population at the 3.0, 6.0, 9.0, and 13.5 μg/kg doses, but the magnitude of the change was smaller than observed for the CD4+FoxP3+CD25bright Tregs. No changes in total CD4+ Tregs in the RUR_{20kD}-IL-2 treated subjects versus placebo subjects were observed at 0.3 μg/kg and 1 μg/kg doses (FIG. 13).

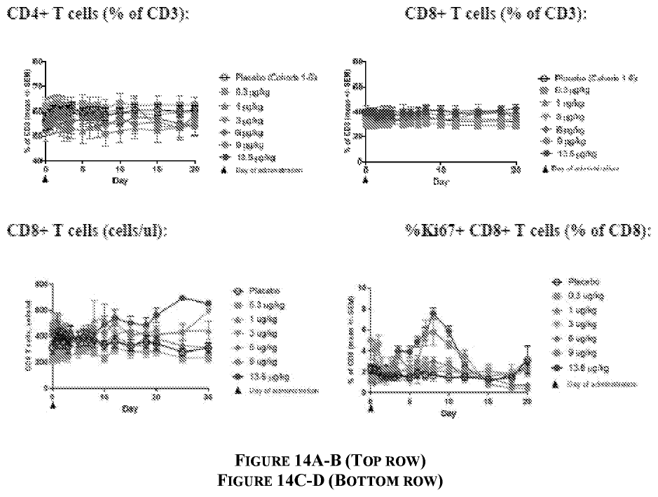

Importantly, the primary effect of RUR_{20kD}-IL-2 was seen on Tregs, as no changes in percentage of Tcon cell populations (CD4+, CD8+) were observed in either the RUR_{20kD}-IL-2 or placebo-treated subjects. However, small increases in the absolute number of CD8+ T cells and the percentage of Ki67+ CD8+ T cells were observed at 13.5 μg/kg in the RUR_{20kD}-IL-2 subjects (FIG. 14A-D). There were no changes in CD4+ T cell absolute numbers at any dose level.

The CD56+ NK cell population was also analyzed. An increase was noted in absolute numbers of circulating NK cells with a similar increase in percentage of this cell subset at the 13.5 μg/kg dose level but not at the lower dose levels. Also noted at 3.0, 6.0, 9.0 and 13.5 μg/kg was a dose-dependent increase in the percentage of CD56+ NK cells expressing Ki67, a marker of proliferation and therefore a marker of activation. At 3.0, 6.0, and 9.0 μg/kg, the percentage expressing Ki67 approximated 10%, 20-30%, and 30-40%, respectively, after RUR_{20kD}-IL-2 administration. There was no further increase in the percentage expressing Ki67 at the 13.5 μg/kg dose, which remained at 30-40%.

RUR_{20kD}-IL-2 treatment according to the SAD study led to a sustained increase in the numbers of CD4+FoxP3+CD25bright Tregs, with levels not returning to baseline until 20-25 days following administration. There also was an increase in the total CD4+FoxP3+CD25+ Treg population, although the magnitude of the change was smaller than observed for the CD4+FoxP3+CD25bright Tregs. Increases in the numbers of CD8+ T cells and NK cells were observed at 13.5 μg/kg.

Additional cohorts of RUR_{20kD}-IL-2, 20.0 mg/kg (n=13); Placebo (n=3), and RUR_{20kD}-IL-2, 28.0 mg/kg (n=9); Placebo (n=3) were also conducted. Each cohort is followed for 50 days to assess the effects of subcutaneous administration of single ascending doses of RUR_{20kD}-IL-2 in healthy volunteers on safety and tolerability in subjects as evaluated by adverse events, vital signs, and clinical laboratory assessments, as well as the time course and extent of changes in the numbers and activity of Tregs, Tcons, and NK cells and subsets, pharmacokinetics of RUR_{20kD}-IL-2, and other immunological effects such as cytokine levels, peripheral blood cell populations, serum proteins and gene expression.

Generally, safety results found no dose-limiting toxicities, deaths, or adverse events leading to study discontinuation, no clinically significant vital sign, ECG, or physical examination abnormalities. Adverse events were primarily limited to mild or moderate (Grade 1 or 2) injection site reactions, 4 subjects who experienced Grade 1 events of headache, 1 subject at the highest dose tested (28.0 μg/kg) who experienced mild (Grade 1) signs and symptoms of pyrexia, anorexia, vomiting, diarrhea, tachycardia, and myalgia (all Grade 1 in severity) attributed to elevated cytokine levels, and no elicitation of anti-drug antibodies.

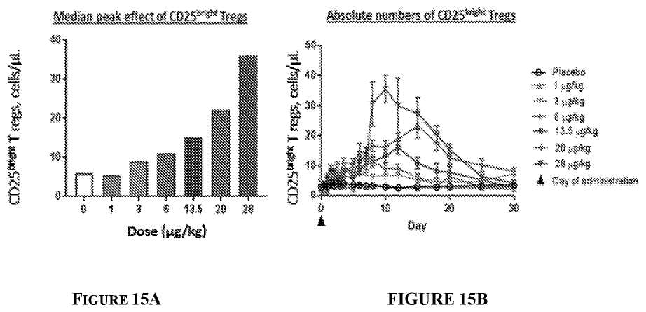

Generally, a sustained, dose-dependent increase in CD25-bright Tregs was observed in response to RUR_{20kD}-IL-2 (See FIG. 15). At 28 μg/kg of an RUR_{20kD}-IL-2 composition, a 17-fold mean peak increase was observed in numbers of CD25-bright Tregs above pre-dose values. Treg levels peak at days 10-12, and do not return to baseline until days 20-25 following administration. Increases in Treg activation markers ICOS and CTLA4 were observed at doses ≥13.5 μg/kg.

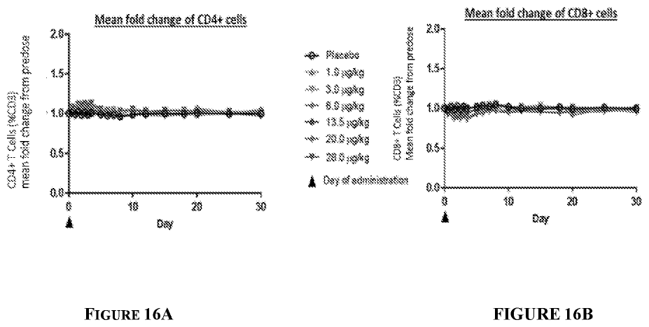

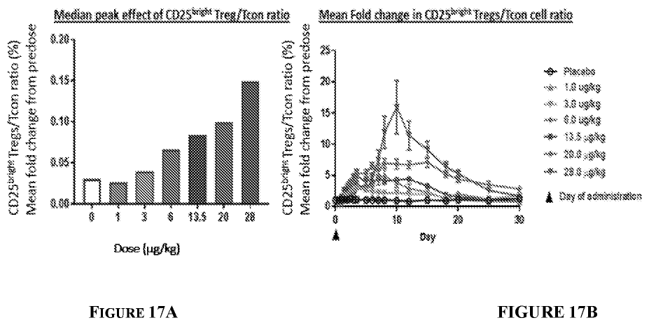

No substantial changes were observed in the percentage of Tcon cells, and minimal increases were observed in CD56+ NK cells in response to RUR_{20kD}-IL-2 (See FIG. 16). (CD16+CD56+ NK cells were also enumerated, data not shown). Increases in NK cells were not dose-dependent. A 2-fold increase in NK cells at highest concentration of RUR_{20kD}-IL-2 was observed. RUR_{20kD}-IL-2 induces dose-dependent increases in Tregs with no induction of CD8+ T cells up to 28 μg/kg. RUR_{20kD}-IL-2 administration leads to 15-fold increase in mean peak Treg:CD8 ratio over baseline at 28 μg/kg. (See FIG. 17).

Study objectives assessed the safety and tolerability of RUR_{20kD}-IL-2 in humans administered single ascending doses subcutaneously (SC). In addition, time course and extent of changes in the numbers and percentages of Tregs, conventional CD4+ and CD8+ T cells, NK cells, cytokine levels, and the pharmacokinetics (PK) of an RUR_{20kD}-IL-2 composition in peripheral blood were investigated. In this first-in-human, double-blind, single ascending dose study, healthy volunteers received SC doses ranging from 0.3 to 28 ug/kg (9 active:3 placebo per cohort) and subjects were followed for 50 days. All 8 planned cohorts completed dosing. There were no dose-limiting toxicities, serious adverse events, deaths, or clinically significant abnormalities in vital signs, electrocardiograms, or laboratory test values. Adverse events attributed to RUR_{20kD}-IL-2 were primarily limited to mild (grade 1) injection site reactions. One subject at the highest dose tested demonstrated transient and mild (grade 1) symptoms of elevated cytokine levels and lymphopenia, which resolved without treatment. No other individual at any dose level had systemic signs or symptoms known to be associated with IL-2 therapy. The first 6 cohorts have been tested for anti-drug antibodies to date and none have been detected. RUR_{20kD}-IL-2 reached maximum plasma levels 4-6 days after administration, with little change for 2 weeks, and then decreased with a half-life of ˜8-9 days. The primary effect of RUR_{20kD}-IL-2 was seen on Tregs. In the 3.0 to 28.0 ug/kg dose cohorts, a dose dependent and sustained increase in the absolute numbers and percentages of circulating CD4+FoxP3+CD25bright Tregs were observed. The elevated levels peaked at Days 10-12 and did not return to baseline until ˜20 to 25 days following administration. At 28.0 ug/kg, the mean peak increase in numbers of these CD25bright Tregs was 17-fold above baseline, and the mean peak percentage increased from 0.5% to 7.4%. In addition, there was an increase in Treg activation markers at doses ≥13.5 ug/kg. There was a mean increase of 3.5-fold in the percentages and numbers of NK cells at the highest dose tested, but no changes in percentages or numbers of conventional CD4+ or CD8+ T cells were observed. an RUR_{20kD}-IL-2 composition selectively induced Tregs, evidenced by a 15-fold increase in the mean peak Treg:CD8 ratio over baseline in the 28.0 ug/kg group. In conclusion, single doses of the IL-2 conjugate T-reg stimulator, RUR_{20kD}-IL-2, in the dose range tested were well tolerated and safe. RUR_{20kD}-IL-2 led to a striking and selective dose-dependent increase in circulating CD25bright Tregs with minimal effects on conventional T cells and with relatively small effects on NK cells. These clinical results extend previous animal studies showing the prolonged and Treg selective action of RUR_{20kD}-IL-2, and provide strong support for testing RUR_{20kD}-IL-2 as a new therapeutic in autoimmune diseases, such as systemic lupus.

An RUR_{20kD}-IL-2 composition was safe and well tolerated in this first in human single ascending dose study, and led to a striking and selective dose-dependent increase in circulating CD25-bright Treg cells. There was minimal effect on Tcons and NK cells, and this study data provides support for testing RUR_{20kD}-IL-2 in autoimmune and inflammatory diseases.

### Example 11

### A Phase I, Double-Blind, Randomized Placebo-Controlled Ascending Multiple-Dose Study to Evaluate the Safety, Tolerability, Pharmacokinetics, and Pharmacodynamics of Subcutaneous RUR_{20kD}-IL-2 in Patients with Systemic Lupus Erythematosus

A double-blind, randomized, placebo-controlled study to evaluate the safety, tolerability, PK, and immunologic effects of ascending multiple doses of RUR_{20kD}-IL-2 in four dose cohorts of patients with minimal to moderate systemic lupus erythematosus (SLE) is performed. The effects on SLE disease activity are also evaluated. Twelve SLE patients with minimal to moderate disease activity are randomized to each of four dose cohorts, nine of whom received multiple 1.0 mg/mL aqueous solution sub-cutaneous doses of RUR-IL-2-20 kD, while three received placebo. RUR_{20kD}-IL-2 drug and placebo are prepared as described herein, for instance as in Example 1-A. Active clinical SLE disease activity is not required as an inclusion criterion. In Cohort 1, a starting dose of 3.0 μg/kg is administered three times at two-week intervals (Days 1, 15, and 29). This starting dose is based on the favorable safety and PD profile of single sub-cutaneous doses of RUR_{20kD}-IL-2 previously determined in the study described above. The subsequent dose levels in Cohorts 2, 3, and 4, respectively, were up to two-fold that of the previous dose cohort. Patients in Cohorts 1-3 received three doses of study drug at two-week intervals over a total of four weeks. Doses to be evaluated over the course of the study range from 3.0 μg/kg to 24 μg/kg. Patients in Cohort 4 receive twelve weeks of treatment with RUR_{20kD}-IL-2, administered on Days 1, 15, 29, 43, 57, 71 and 85. This cohort provides data on the safety of administration and PK and PD profiles over a longer duration of RUR_{20kD}-IL-2 treatment. After receiving the final dose of RUR_{20kD}-IL-2 or placebo, patients are followed for an additional fifty days to evaluate safety, PK, PD, and preliminary efficacy. Eight of twelve subjects in each cohort are evaluated two weeks after the third dose of the final patient by the Safety Review Committee for possible safety issues. In addition, all patients in Cohort 4 are evaluated by the Safety Review Committee twice: (1) two weeks after the first eight subjects receive their third dose and (2) two weeks after all subjects receive all doses of study drug. Immunologic changes, including Tregs, CD4⁺-T cells, CD8⁺-T cells, and NK cell responses, cytokine levels, and available PK data, in addition to safety findings, are used to determine dose levels. The primary objective of the study is to evaluate the safety and tolerability of RUR_{20kD}-IL-2 administered as multiple ascending subcutaneous doses to patients with SLE. The secondary objectives of the study are to (1) characterize the PK profile of RUR_{20kD}-IL-2 following multiple sub-cutaneous doses in patients with SLE, (2) assess the effects of RUR_{20kD}-IL-2 on the time course and extent of changes in PD biomarkers, including number and function of Tregs and Treg subsets, CD4⁺-T cells, CD8⁺-T cells, NK cells, and cytokine levels in patients with SLE, (3) assess the effects of RUR_{20kD}-IL-2 on the presence and levels of antibodies against double-stranded DNA, and levels of complement C3 and C4 in patients with SLE, and (4) assess effects of RUR_{20kD}-IL-2 on disease activity in SLE patients. Results depicting preliminary PK data from the ascending multi-dose study are compared with data from the single subcutaneous study in the below Table 15:

TABLE 15
PK Data in Single and Multi-Dose Human Studies

| | Single Dose Study | | | | Multi-Dose Study | | | |
|---|---|---|---|---|---|---|---|---|
| | | AUC_{0-14 d} | Cₘₐₓ | tₘₐₓ | | AUC_{0-14 d} | Cₘₐₓ | tₘₐₓ |
| Dose | | (ng/mL*day) | (ng/mL) | (day) | | (ng/mL*day) | (ng/mL) | (day) |
| 3.0 | Median | 214 | 21 | 3.5 | Median | 185 | 18 | 6 |
| μg/kg | (n = 9) | | | | (n = 9) | | | |
| | min | 143 | 13 | 1.3 | min | 113 | 15 | 2 |
| | max | 388 | 41 | 14 | max | 291 | 26 | 14 |
| 6.0 | Median | 542 | 46 | 5 | Median | 113 | 14 | 6 |
| μg/kg | (n = 9) | | | | (n = 7) | | | |
| | min | 73 | 13 | 2 | min | 43 | 5 | 4 |
| | max | 705 | 70 | 9 | max | 206 | 22 | 14 |

1. A composition comprising PEGylated IL-2 conjugates of the formula: 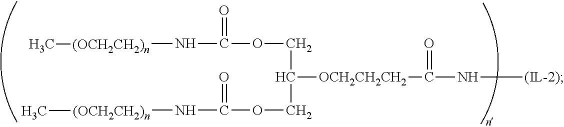
wherein:
IL-2 is an interleukin-2;
n is independently at each occurrence an integer from about 3 to about 4000; and
n′ is 1 and 2 and 3.

2. The composition of claim 1, wherein IL-2 is aldesleukin.

3. The composition of claim 2, wherein the composition comprises no more than about 20 mole percent of PEGylated IL-2 conjugates, when considered collectively, encompassed b the formula 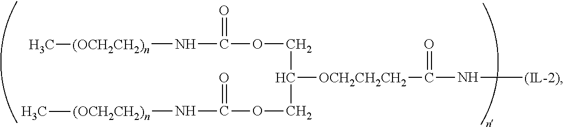
wherein n′ is selected from 1, 4, 5, or an integer greater than 5.

4. The composition of claim 3, wherein the composition comprises no more than about 15 mole percent of PEGylated IL-2 conjugates, when considered collectively, encompassed by the formula 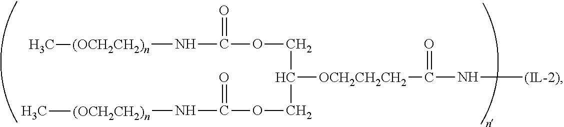
wherein n′ is selected from 1, 4, 5, or an integer greater than 5.

5. The composition of claim 3, wherein the composition comprises no more than about 10 mole percent of PEGylated IL-2 conjugates, when considered collectively, encompassed b the formula 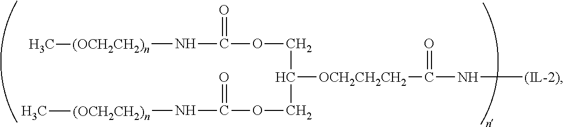
wherein n′ is selected from 1, 4, 5, or an integer greater than 5.

6. The composition of any one of claims 3-5, comprising no more than about 10 mol % of PEGylated IL-2 conjugates having n′ equal to 1.

7. The composition of any one of claims 3-5, comprising no more than about 7 mol % of PEGylated IL-2 conjugates having n′ equal to 1.

8. The composition of any one of claims 3-5, comprising no more than about 5 mol % of PEGylated IL-2 conjugates having n′ equal to 1.

9. The composition of claim 3, comprising no more than about 10 mol % of PEGylated IL-2 conjugates having n′ equal to 4.

10. The composition of claim 3, comprising no more than about 7 mol % of PEGylated IL-2 conjugates having n′ equal to 4.

11. The composition of claim 3, comprising no more than about 5 mol % of PEGylated IL-2 conjugates having n′ equal to 4.

12. A composition comprising a mixture of PEGylated IL-2 conjugates of claim 1, wherein the composition comprises approximately equimolar amounts of

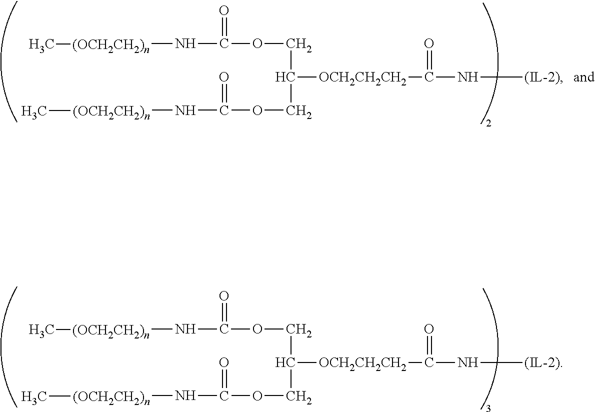

13. A composition comprising a mixture of PEGylated IL-2 conjugates of claim 2, wherein the composition comprises PEGylated IL-2 conjugates having a formula 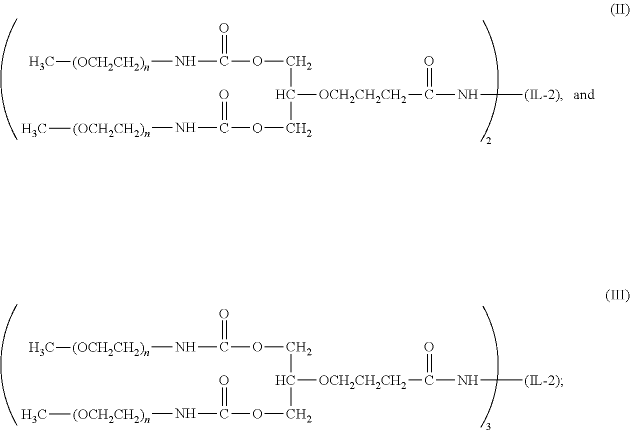
wherein the molar ratio of (H)/(TT) is selected from the group consisting of 1.4:1; 1.3:1; 1.2:1; 1.1:1; 1:1; 1:1.1; 1:1.2; 1:1.3; and 1:1.4.

14. The composition of claim 13, having an average number of branched polyethylene glycol moieties, 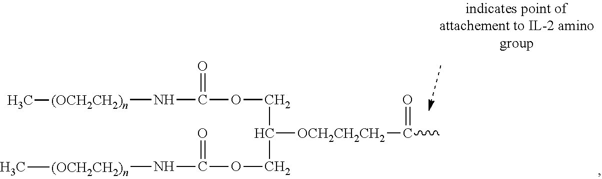
per aldesleukin is selected from the group consisting of 2; 2.1; 2.2; 2.3; 2.4; 2.5; 2.6; 2.6; 2.7; 2.8; 2.9; and 3.

15. The composition of claim 13, wherein the average number of branched polyethylene glycol moieties per aldesleukin is about 2.5.

16. The composition of claim 6, wherein the value of n ranges from 5-2000.

17. The composition of claim 6, wherein the value of n ranges from 10-1000.

18. The composition of claim 6, wherein the value of n ranges from 10-750.

19. The composition of claim 6, wherein the value of n ranges from 10-500.

20. The composition of claim 6, wherein the value of n ranges from 20-250.

21. The composition of claim 6, wherein the average value of n is about 226.

22. The composition of claim 6, wherein the nominal average molecular weight of each branched polyethylene glycol moiety is in a range of from about 250 daltons to about 90,000 daltons.

23. The composition of claim 6, wherein the nominal average molecular weight of each branched polyethylene glycol moiety is in a range of from about 1000 daltons to about 60,000 daltons.

24. The composition of claim 6, wherein the nominal average molecular weight of each branched polyethylene glycol moiety is in a range of from about 5,000 daltons to about 60,000 daltons.

25. The composition of claim 6, wherein the nominal average molecular weight of each branched polyethylene glycol moiety is in a range of from about 10,000 daltons to about 55,000 daltons.

26. The composition of claim 1 or 2 comprising, on a molar basis, about 5 mol % or less mono-PEGylated IL-2 conjugates, and from about 28 mol % to about 60 mol % di-PEGylated IL-2 conjugates, and from about 24 mol % to about 65 mol % tri-PEGylated IL-2 conjugates, and about 12 mol % or less of higher PEGylated IL-2 conjugates, and wherein the nominal average molecular weight of each branched polyethylene glycol moiety is about 20,000 daltons.

27. The composition of claim 26 which further comprises 80% or greater combined di- and tri-PEGylated IL-2 conjugates.

28. The composition of claim 1 or 2 comprising, on a molar basis, from about 2.5 to about 4.5 mol % mono-PEGylated IL-2 conjugates, and from about 35 to about 50 mol % di-PEGylated IL-2 conjugates, and from about 38 to about 46 mol % tri-PEGylated IL-2 conjugates, and from about 3 to about 10 mol % higher PEGylated IL-2 conjugates, and wherein the nominal average molecular weight of each branched polyethylene glycol moiety is about 20,000 daltons.

29. The composition of claim 28 which further comprises a combined total of di-PEGylated and tri-PEGylated IL-2 conjugates from about 80 to about 95 mol %.

30. The composition of claim 1 or 2 comprising, on a molar basis, from about 2.8 to about 3.8 mol % mono-PEGylated IL-2 conjugates, and from about 44 to about 48 mol % di-PEGylated IL-2 conjugates, and from about 41 to about 44 mol % tri-PEGylated IL-2 conjugates, and from about 7 to about 9 mol % higher PEGylated IL-2 conjugates, and wherein the nominal average molecular weight of each branched polyethylene glycol moiety is about 20,000 daltons.

31. The composition of claim 30 which further comprises a combined total of di-PEGylated and tri-PEGylated IL-2 conjugates from about 87 to about 90 mol %.

32. The composition of claim 1 or 2 comprising, on a molar basis, from about 2.8 to about 3.8 mol % mono-PEGylated IL-2 conjugates, and from about 44 to about 48 mol % di-PEGylated IL-2 conjugates, and from about 41 to about 44 mol % tri-PEGylated IL-2 conjugates, and from about 7 to about 9 mol % higher PEGylated IL-2 conjugates, and wherein said composition comprises a mixture of mono-PEGylated IL-2 conjugates which have a PEG moiety attached at one of lysine K7 or K8 or K31 or K75, and wherein the nominal average molecular weight of each branched polyethylene glycol moiety is about 20,000 daltons.

33. The composition of claim 32 which further comprises a combined total of di-PEGylated and tri-PEGylated IL-2 conjugates from about 87 to about 90 mol %.

34. The composition of claim 1 or 2 comprising, on a molar basis, from about 2.8 to about 3.8 mol % mono-PEGylated IL-2 conjugates, and from about 44 to about 48 mol % di-PEGylated IL-2 conjugates, and from about 41 to about 44 mol % tri-PEGylated IL-2 conjugates, and from about 7 to about 9 mol % higher PEGylated IL-2 conjugates, and wherein said composition comprises mono-PEGylated IL-2 conjugates which have a PEG moiety attached at lysine K7, wherein the nominal average molecular weight of each branched polyethylene glycol moiety is about 20,000 daltons.

35. The composition of claim 34 which further comprises a combined total of di-PEGylated and tri-PEGylated IL-2 conjugates from about 87 to about 90 mol %.

36. The composition of any one of claims 26-35, further comprising a pharmaceutically acceptable excipient.

37. The composition of any one of claims 26-35, in a form suitable for parenteral administration.

38. The composition of any one of claims 26-35, in a form suitable for subcutaneous administration.

39. The composition of claim 36, comprising an aqueous diluent.

40. The composition of claim 39, having a pH of about 5.

41. The composition of claim 40, further comprising sodium acetate, sodium chloride and sucrose.

42. The composition of claim 37, comprising 1.5 mg/ml protein equivalent, 10 mM sodium acetate, 110 mM sodium chloride, 2% sucrose (w/v), pH 5.0.

43. A method of increasing the ratio of regulatory T cells to effector T cells in a subject by administering to the subject a therapeutically effective dose of a composition of any one of claims 26-35.

44. The method of claim 43, wherein the regulatory T cells are selected from Foxp3+ and CD25+ cells.

45. The method of claim 44, wherein the effector T cells are selected from CD4+ and CD8+ cells.

46. The method of claim 43, wherein the fold-increase in regulatory T cells, when compared to baseline, reaches a value of at least about 2 when evaluated in an in-vivo mouse model.

47. The method of claim 43, wherein the fold increase in regulatory T cells, when compared to baseline, reaches a value of at least about 4 when evaluated in an in-vivo mouse model.

48. The method of claim 43, wherein the increase in regulatory T cell numbers is sustained above baseline levels for at least 3 days post-administration.

49. The method of claim 43, wherein the increase in regulatory T cell numbers is sustained above baseline levels for at least 5 days post-administration.

50. A method of treating a subject having an autoimmune disease, comprising administering to the subject a therapeutically effective amount of a composition of claim 26.

51. The method of claim 50, wherein said administering is by subcutaneous injection.

52. The method of claim 50, wherein said administering is carried out once every 2 weeks or once every 4 weeks.

53. The method of claim 50, wherein said administering comprises a dose of between 3-24 μg/kg once every two weeks.

54. The method of claim 50, wherein said autoimmune disease is systemic lupus erythematosus.

55. The method of claim 50, wherein said autoimmune disease is atopic dermatitis.

56. The method of claim 50, wherein said autoimmune disease is ulcerative colitis or Crohn's disease.

57. A method of treating a subject having an allergic disease, comprising administering to the subject a therapeutically effective amount of a composition of claim 26.

58. A method of treating a subject having peanut allergy, comprising administering to the subject a therapeutically effective amount of a composition of claim 26.
